SEQUENCE LISTING (46 sequences)

SEQ ID NO: 1 - protein, 57 aa, synthetic construct
YLGLEWKTKTVPYLGVEWRTKTVSYWFFGWHTKQVAYLAPVWKEKTIPYAVPVTLSK

SEQ ID NO: 2 - protein, 691 aa, Vibrio cholerae
MKQTKTLTAISVLALSHLMTQSTAFASSSSDIQTKLKWSWSTSVFHPESNQVMAAPIVVQLNDDNGDGKIDEKDVADIIVVTFEGNKYANGGYIRALSGVDGSELWSYSNGGVIADARYAPAAADLDGDGLIEIVSTSALTPYINILDHQGNIKKQLLKSASGWRSVGDIALADINGDGNIEILAADGVYSYESGLLFSHDWAPSSIAFDSNGDGQREVFANGTLYQNNGAYLWQYQANDTVWFSSVANLDGDDKPELVVSVPASLSTPENSEIAVLEHDGSVKWRVNNLSNPGGSVQAVSSFLGKPSSSATTVDAQSAVYGYTDWAHQQRVLAENHQLAIRSGAVVDAIGANSQNMIGGSGGSLSTIDTSKVRAIDVTYGKNKYTWKYGVLEMSFTLDNGAKVTVGSKDSAFTYLGLEWKTKTVPYLGVEWRTKTVSYWFFGWHTKQVAYLAPVWKEKTIPYAVPVTLSKSTTVRYDIPQGSQLLGMNVWSKEKHLFKHKQQVNAVQFLVGKVTADQSHMGIVYAGYYAVDMYDAQGNKVWSVANDDLNSGKIGVSAYDFTGDGIDEVLVQDRLRMRILDGQTGRVMGIIANSSGTLWEYPVVADLEGNNNASLIMVANDYDRESQVNHGVFVYESANPSKPWRNATRIWNQYAFNFSDINANGTIPTNAQPSWLTHNSFRSATIRVPLK

SEQ ID NO: 3 - protein, 200 aa, synthetic construct
DAQSAVYGYTDWAHQQRVLAENHQLAIRSGAVVDAIGANSQNMIGGSGGSLSTIDTSKVRAIDVTYGKNKYTWKYGVLEMSFTLDNGAKVTVGSKDSAFTYLGLEWKTKTVPYLGVEWRTKTVSYWFFGWHTKQVAYLAPVWKEKTIPYAVPVTLSKSTTVRYDIPQGSQLLGMNVWSKEKHLFKHKQQVNAVQFLVGKV

SEQ ID NO: 4 - protein, 57 aa, synthetic construct
YPALEWRTKEVPYLGLEWRTKQVSYWFFGWHTKTVSYLAPVWKTKTIPYAVPVMKSK

SEQ ID NO: 5 - protein, 689 aa, Vibrio anguillarum
MTSKFSLCAVGLLSISSIAVSTIATASNPSEINTQLKWSWESSSFKPESNQVMAAPVIAQLNDDNGDGKIDENDIADIIVVTFENNKYTQGGLVRALSGIDGSELWSYDNGGIIADARYSPAVADLDGNGVVDIVITSASSPYITILDNEGNIKKQILKHVTGGRSVGTISISDLNNDGSIEIISADGVYNYDTGLLFSLEWAPSSISFDADGDGVQEIFSNGALYKSDGSFTWQYQANDTVWFSSVANLDSDNKPEIVVSVPATKATAQNSVFAVLEHDGSVKWEVNNLENPGGGVQAISNFLGNTATSSTNEIAKSPVYGYTHLHHSHPVKIADDNQLKIRSGDLIDAIGSTASNMVGGQGGSLHTIDASKVRSVDVTYGKYKTWWTYGVLEMEFTLNDGSKITLGSKDSAFKYPALEWRTKEVPYLGLEWRTKQVSYWFFGWHTKTVSYLAPVWKTKTIPYAVPVMKSKATTERYTVPSNTQLVGLNVWSKPKPIFTFKKHVNAVQFVVGESINDSYLNTGIVYAGYHAVDMYNAQGSKVWSVANDDYNSGKIGVSAYDFTGDGIDEVIVQDLLRVRILDGRTGAVLATIANSSNTLWEYPVVADLEGNNNASLIVVANDYAKESAINHGVYVYESADADKPWKNATRIWNQHSFHFSNINQDGSVPTNAQPSWLTHNTYRSSTIK

SEQ ID NO: 6 - protein, 201 aa, synthetic construct
IAKSPVYGYTHLHHSHPVKIADDNQLKIRSGDLIDAIGSTASNMVGGQGGSLHTIDASKVRSVDVTYGKYKTWWTYGVLEMEFTLNDGSKITLGSKDSAFKYPALEWRTKEVPYLGLEWRTKQVSYWFFGWHTKTVSYLAPVWKTKTIPYAVPVMKSKATTERYTVPSNTQLVGLNVWSKPKPIFTFKKHVNAVQFVVGES

SEQ ID NO: 7 - protein, 957 aa, Vibrio cholerae
MTSHYIALAVGLLSLSSNVVQATTNEAEGCIISRLNGEKYCLKVGERSGYSLPSWIYAHPVDVQAPSGVSVMLSDWDNLSYNRLAVFDRYTGNEDLKNVKAYNGAYLDFSKPRSMRVLASETYPEACIVSRQTGERFCLKEGERSGYSLPAYIYGHEVDVEAPLGLGVMLSDWDNLSYNRLAVFGGNTQNEQMRAVKAYNGETLDFSKPRSMRVVPYDGDSSALNMKLKWSWQGSAFQPNSNQVMVTPIVAQLNDDNGDGKIDEKDVADLIVVTFEGNKYANGGLVRALSGVDGSELWSYANGGVIADARYSPAVGDLDGDGIVEIVTTNNRDQFITILDNQGNIKKQIPTTESGWRIVGDITLADLDHDGSVEILAADGVYNYHSGLVFNHPWAPSSINVDVDGDQQQEVFSGGTLFQNNGAINWQYQANDAVWFSSLVNLDNDAEPEIVASVPATFATGDNARFAVLEHDGTIKWEINNTANPGGGVQAVSNFLGKAQAVETSEFSKVYGYQPNNNPASIALAVDGKISVRSGFAIDAIGASASTLVGGTGGNLNAAVNVKDIKAIDLTWGKYYWGGYHLLALDFRMSNGSVISMGSKNYAYSKQTERFTVPAGSRIKGIKAWTAGWLLDGVQFELATQNGTNDLDVKGIVYAGYAAVDMYNSKGERVWSVANDDTGSGKIGVSAYDFDNDGIDEVLVQDHARVRVLDGKTGKERASLAHSTATLWEYPIVVDLEGDNNAELIVAANDFDRQYSINHGVYVYQSADSSKPWKNATRIWNQHAFHLTNINQDGTLPTFVEPSWLSHNTYRSSTLRAAVGGESPIFGYSNTQQSQRVVTADNLMYLRSGFAIDAIGTTVNNLVGGPVQGTNGGVLRAPIALDQLQSVEVTSGLYNWGGYHIVAIKFTMKDGSSVLLGSTHYASNKKVETYTVPQGKRIKQINVWTGGWLVEGFQFVY

SEQ ID NO: 8 - protein, 8 aa, synthetic construct
DYKDDDDK

SEQ ID NO: 9 - protein, 6 aa, synthetic construct
YPYDVP

SEQ ID NO: 10 - protein, 10 aa, synthetic construct
ILKKATAYIL

SEQ ID NO: 11 - protein, 10 aa, synthetic construct
EQKLISEEDL

SEQ ID NO: 12 - protein, 7 aa, synthetic construct
GQSSRSS

SEQ ID NO: 13 - protein, 6 aa, synthetic construct
GSGSGS

SEQ ID NO: 14 - protein, 6 aa, synthetic construct
ASGGGS

SEQ ID NO: 15 - protein, 10 aa, synthetic construct
GGGGSGGGGS

SEQ ID NO: 16 - protein, 20 aa, synthetic construct
GGGGSGGGGSGGGGSGGGGS

SEQ ID NO: 17 - protein, 15 aa, synthetic construct
GGGGSGGGGSGGGGS

SEQ ID NO: 18 - protein, 6 aa, synthetic construct
LVPRGS

SEQ ID NO: 19 - protein, 7 aa, synthetic construct
  VARIANT 7: note = X = S or G or A or M or C or H
ELNYFQX

SEQ ID NO: 20 - protein, 666 aa, synthetic construct
MKMIQRPLNWLVLAGAATGFPLYAAQMVTIDDASMVEQALAQQQYSMMPAASGFKAVNTVQLPNGKVKVRYQQMYNGVPVYGTVVVATESSKGISQVYGQMAQQLEADLPTVTPDIESQQAIALAVSHFGEQHAGESLPVENESVQLMVRLDDNQQAQLVYLVDFFVASETPSRPFYFISAETGEVLDQWDGINHAQATGTGPGGNQKTGRYEYGSNGLPGFTIDKTGTTCTMNNSAVKTVNLNGGTSGSTAFSYACNNSTNYNSVKTVNGAYSPLNDAHFFGKVVFDMYQQWLNTSPLTFQLTMRVHYGNNYENAFWDGRAMTFGDGYTRFYPLVDINVSAHEVSHGFTEQNSGLVYRDMSGGINEAFSDIAGEAAEYFMRGNVDWIVGADIFKSSGGLRYFDQPSRDGRSIDHASQYYSGIDVHHSSGVFNRAFYLLANKSGWNVRKGFEVFAVANQLYWAPNSTFDQGGCGVVKAAQDLNYNTADVVAAFNTVGVNASCGTTPPPVGKVLEKGKPITGLSGSRGGEDFYTFTVTNSGSVVVSISGGTGDADLYVKAGSKPTTSSWDCRPYRSGNAEQCSISAVVGTTYHVMLRGYSNYSGVTLRLDYLGLEWKTKTVPYLGVEWRTKTVSYWFFGWHTKQVAYLAPVWKEKTIPYAVPVTLSK

SEQ ID NO: 21 - protein, 592 aa, synthetic construct
MFKKFLSLCIVSTFSVAATSALAQPNQLVGKSSPQQLAPLMKAASGKGIKNQYIVVLKQPTTIMSNDLQAFQQFTQRSVNALANKHALEIKNVFDSALSGFSAELTAEQLQALRADPNVDYIEQNQIITVNPIISASANAAQDNVTWGIDRIDQRDLPLNRSYNYNYDGSGVTAYVIDTGIAFNHPEFGGRAKSGYDFIDNDNDASDCQGHGTHVAGTIGGAQYGVAKNVNLVGVRVLGCDGSGSTEAIARGIDWVAQNASGPSVANLSLGGGISQAMDQAVARLVQRGVTAVIAAGNDNKDACQVSPAREPSGITVGSTTNNDGRSNFSNWGNCVQIFAPGSDVTSASHKGGTTTMSGTSMASPHVAGVAALYLQENKNLSPNQIKTLLSDRSTKGKVSDTQGTPNKLLYSLTDNNTTPNPEPNPQPEPQPQPDSQLTNGKVVTGISGKQGELKKFYIDVPAGRRLSIETNGGTGNLDLYVRLGIEPEPFAWDCASYRNGNNEVCTFPNTREGRHFITLYGTTEFNNVSLVARYYLGLEWKTKTVPYLGVEWRTKTVSYWFFGWHTKQVAYLAPVWKEKTIPYAVPVTLSK

SEQ ID NO: 22 - protein, 328 aa, synthetic construct
MSNFKGSIMNKRHYYLASCLALLFSTASYAEVDCELQPVIEANLSLNQNQLASNGGYISSQLGIRNESCETVKFKYWLSIKGPEGIYFPAKAVVGVDTAQQESDALTDGRMLNVTRGFWVPEYMADGKYTVSLQVVAENGKVFKANQEFVKGVDLNSLPELNGLTIDIKNQFGINSVESTGGFVPFTVDLNNGREGEANVEFWMTAVGPDGLIIPVNAREKWVIASGDTYSKVRGINFDKSYPAGEYTINAQVVDIVSGERVEQSMTVVKKYLGLEWKTKTVPYLGVEWRTKTVSYWFFGWHTKQVAYLAPVWKEKTIPYAVPVTLSK

SEQ ID NO: 23 - protein, 465 aa, synthetic construct
MLLYLNQFNKEGGILRYIFISLVLFSPAIFSEVNSHNVIEYGAIANDGEDDSNAFQHALNQLNNGDALIIPTGEYQICKTLYLKEKNNIEIIGSINSKLKKCRSFNGEYLLHITYTQNLKIQGLSFEGLNNGDLKPLWGEQGVYLGSTKGTLVVQNQFARFGDAALRMTTASQDHSIPPGSMAIKVSHNHFEDCAQVTTTQATAGTEMHGTQDIIIDNNQFNACKLKLSARADTRGAKVINNQFENINGTSNEVSYYSDVYYSGNTFLNINGFAINIYPNSRTEQNVQWGNISIIGNTFDAIQQGIRLQSFSINDPNNQSIKNIQISDNTFENIYFGNEIESQYKAIIRTNSQDNLVSFEHVNITGNQYQLTPYSKFISIDHKSKLINIQNNERIYSDHYYIQHFIKDYLGLEWKTKTVPYLGVEWRTKTVSYWFFGWHTKQVAYLAPVWKEKTIPYAVPVTLSK

SEQ ID NO: 24 - protein, 288 aa, synthetic construct
MMIFRFVTTLAASLPLLTFAAPISFSHAKNEAVKIYRDHPVSFYCGCEIRWQGKKGIPDLESCGYQVRKNENRASRIEWEHVVPAWQFGHQLQCWQQGGRKNCTRTSPEFNQMEADLHNLTPAIGEVNGDRSNFSFSQWNGVDGVTYGQCEMQVNFKERTAMPPERARGAIARTYLYMSEQYGLRLSKAQSQLMQAWNNQYPVSEWECVRDQRIEKVQGNSNRFVREQCPNYLGLEWKTKTVPYLGVEWRTKTVSYWFFGWHTKQVAYLAPVWKEKTIPYAVPVTLSK

SEQ ID NO: 25 - protein, 926 aa, synthetic construct
MEMRTTPNLTRSTLALAISFGLVAPSYADLLISQYVEGSSFNKAIEIANTSDQTVSLNGYQLAMSTNGSGTWDKTLPLDGQVIAARDVLVIAHGSANSAILATADLTNNTVVNFNGNDPIALLNSDGSVHDVVGSMGGADFAKDNTLARTTLTPSATYQASDWATQGKDNIDGLGALDTTTPPSAFNCTLDGAEPSFTTIQQIQGEGSTSPYIQGYPYITNEDFFVKGVVSAVTTGLTKGFYLQSLEDDYNPNTSEGLFVFTNQSSSDLAPGDVVCVKGKVQEYYNLTQLKAENNQWVKQGQQAAPQAQAIEILPSDEHFAQTLERYEGMLVKTTPELDMRVTRTFGYDYASRRNNMVLAQGRINMQPNQQHPAGSEQASQQKLDNAQRRLFVESDAKAPDGQIPYYPTFGRTDVDQDGSTEDYIRIDDTVSGLEGVVSYSYNEYRLIVTNTISAENLVHNAPRQAKPDLDEGDLRIATFNVLNYFNSPFGGDANQHGDNRGANNLAEFEVQQAKIVNAIVRLDADIVGLMEIENNGFGEGSAIAQLVNQINSQIADKKKHYRFVAIDSNGDGKTDAADSLGTDVITTGVIYRDKVVKLAQNRVIPMPSQQAPEVVDANGKVIEDGKNYQRDTLAPTFKVKGGNEKITVAVNHFKSKGSACWEDAAPVEQGGQAGKDLDYQGACENFRVAAAVALGDALAKIDGHKVILGDMNSYGMEDPMLVLTDYTPEKYGKTIRAARNTYIAGLEQFGDAGAEIKHSYGYLNAVAMKHPDSWSYSFNDEVGALDHLLVSPSLKHKVVDATDWHINGAESTLFDYNDEFKGNLPKYKDQFRASDHDPAVLELNIYGGSLGLGALLGLLGLGVWRRRRYLGLEWKTKTVPYLGVEWRTKTVSYWFFGWHTKQVAYLAPVWKEKTIPYAVPVTLSK

SEQ ID NO: 26 - protein, 903 aa, synthetic construct
MKRYCLAAVITASLGVSYSAQAYNCAGVPVWDSSTVYVGSDKVQKTNTAYQARYWTQGNDPVTHSGQWDAWQILGQCDGGANNPPQVSIQSPLNNAKIPQGSVVGLQANASDSDGSITQVEFLVGTQRIAIDQQAPYQVDWTATLGATSVTAIATDNQGATTSSTVNISVTPTGNPVPPTVTLTSPTGSEQLTVGDVLAVAANATDSDGTVNAVEFYVDGQLVVIDSSEPYQFNWNAAVGSHTFKAKAIDNDNLSTLSQEVTLTVGSGSNAGCAGLPVYSVGTAYSAGQLVQNKNQKYRCDIAGWCSSSSGWAYEPGVGSYWKEAWSGLGACSTPPVVTLTNPTANQVILAGSTVSVAAQASDADGSVMQVEFFAGNNSLGVVTQAPYAVNWLATTTGNQTLKAVATDNDSNTSESAVSVTVSDQDLVVSLTSPTSGQTVGLGKPVNIAADATSLTNNVAKVEFVVNGAVVATDTTEPFAYSWTPSAIGNYTVAAKATDAAGTSVTSSAAAISVVEQAQKKHRLIGYWHNFVNGAGCPIRLADMSQAWDVIDIAFAENDRNSTGTVHFNLYAGDIYSSCPALDPAQFKQDMKALQAKGKVFVLSLGGAEGTITLNTDQDEANFVSSLTALIKEWGFDGLDVDLESGSNLVHGSQIQARLGRALKQIEKNIGGDMFLTMAPEHPYVQGGMVAYSGIWGAYIPVINEVRDTLDILHVQLYNNGGLPNPYTPSAAPEGSVDMMVAQSKMLIEGFTLANGTRFEPLRDDQVAIGLPSGPSSANSGQAPTQNILDALDCLTKGTRCGTIKPAFAYPNYAGVMTWSINWDKHDGFNFSKPVGDKLSQMNNAQYLGLEWKTKTVPYLGVEWRTKTVSYWFFGWHTKQVAYLAPVWKEKTIPYAVPVTLSK

SEQ ID NO: 27 - protein, 1014 aa, synthetic construct
MTSHYIALAVGLLSLSSNVVQATTNEAEGCIISRLNGEKYCLKVGERSGYSLPSWIYAHPVDVQAPSGVSVMLSDWDNLSYNRLAVFDRYTGNEDLKNVKAYNGAYLDFSKPRSMRVLASETYPEACIVSRQTGERFCLKEGERSGYSLPAYIYGHEVDVEAPLGLGVMLSDWDNLSYNRLAVFGGNTQNEQMRAVKAYNGETLDFSKPRSMRVVPYDGDSSALNMKLKWSWQGSAFQPNSNQVMVTPIVAQLNDDNGDGKIDEKDVADLIVVTFEGNKYANGGLVRALSGVDGSELWSYANGGVIADARYSPAVGDLDGDGIVEIVTTNNRDQFITILDNQGNIKKQIPTTESGWRIVGDITLADLDHDGSVEILAADGVYNYHSGLVFNHPWAPSSINVDVDGDQQQEVFSGGTLFQNNGAINWQYQANDAVWFSSLVNLDNDAEPEIVASVPATFATGDNARFAVLEHDGTIKWEINNTANPGGGVQAVSNFLGKAQAVETSEFSKVYGYQPNNNPASIALAVDGKISVRSGFAIDAIGASASTLVGGTGGNLNAAVNVKDIKAIDLTWGKYYWGGYHLLALDFRMSNGSVISMGSKNYAYSYLGLEWKTKTVPYLGVEWRTKTVSYWFFGWHTKQVAYLAPVWKEKTIPYAVPVTLSKKQTERFTVPAGSRIKGIKAWTAGWLLDGVQFELATQNGTNDLDVKGIVYAGYAAVDMYNSKGERVWSVANDDTGSGKIGVSAYDFDNDGIDEVLVQDHARVRVLDGKTGKERASLAHSTATLWEYPIVVDLEGDNNAELIVAANDFDRQYSINHGVYVYQSADSSKPWKNATRIWNQHAFHLTNINQDGTLPTFVEPSWLSHNTYRSSTLRAAVGGESPIFGYSNTQQSQRVVTADNLMYLRSGFAIDAIGTTVNNLVGGPVQGTNGGVLRAPIALDQLQSVEVTSGLYNWGGYHIVAIKFTMKDGSSVLLGSTHYASNKKVETYTVPQGKRIKQINVWTGGWLVEGFQFVY

SEQ ID NO: 28 - protein, 798 aa, synthetic construct
MPKLNRCAIAIFTILSAISSPTLLANINEPSGEAADIISQVADSHAIKYYNAADWQAEDNALPSLAELRDLVINQQKRVLVDFSQISDAEGQAEMQAQFRKAYGVGFANQFIVITEHKGELLFTPFDQAEEVDPQLLEAPRTARLLARSGFASPAPANSETNTLPHVAFYISVNRAISDEECTFNNSWLWKNEKGSRPFCKDANISLIYRVNLERSLQYGIVGSATPDAKIVRISLDDDSTGAGIHLNDQLGYRQFGASYTTLDAYFREWSTDAIAQDYRFVFNASNNKAQILKTFPVDNINEKFERKEVSGFELGVTGGVEVSGDGPKAKLEARASYTQSRWLTYNTQDYRIERNAKNAQAVSFTWNRQQYATAESLLNRSTDALWVNTYPVDVNRISPLSYASFVPKMDVIYKASATETGSTDFIIDSSVNIRPIYNGAYKHYYVVGAHQSYHGFEDTPRRRITKSASFTVDWDHPVFTGGRPVNLQLASFNNRCIQVDAQGRLAANTCDSQQSAQSFIYDQLGRYVSASNTKLCLDGEALDALQPCNQNLTQRWEWRKGTDELTNVYSGESLGHDKQTGELGLYASSNDAVSLRTITAYTDVFNAQESSPILGYTQGKMNQQRVGQDHRLYVRAGAAIDALGSASDLLVGGNGGSLSSVDLSGVKSITATSGDFQYGGQQLVALTFTYQDGRQQTVGSKAYVTNYLGLEWKTKTVPYLGVEWRTKTVSYWFFGWHTKQVAYLAPVWKEKTIPYAVPVTLSKAHEDRFDLPAAAKITQLKIWSDDWLVKGVQFDLN

SEQ ID NO: 29 - protein, 432 aa, synthetic construct
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCFYLGLEWKTKTVPYLGVEWRTKTVSYWFFGWHTKQVAYLAPVWKEKTIPYAVPVTLSK

SEQ ID NO: 30 - protein, 508 aa, synthetic construct
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFSVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRAHFPLATYAPVISAEKAYHEQLSVAEITNACFEPANQMVKCDPRHGKYMACCLLYRGDVVPKDVNAAIATIKTKRTIQFVDWCPTGFKVGINYEPPTVVPGGDLAKVQRAVCMLSNTTAIAEAWARLDHKFDLMYAKRAFVHWYVGEGMEEGEFSEAREDMAALEKDYEEVGVDSVEGEGEEEGEEYYLGLEWKTKTVPYLGVEWRTKTVSYWFFGWHTKQVAYLAPVWKEKTIPYAVPVTLSK

SEQ ID NO: 31 - protein, 1343 aa, synthetic construct
MGSSHHHHHHSSGLVPRGSHMASMTGGQQMGRDPSGLNGANAELTIHESNLSGGSSPDHSALSKVGSFSFTSIDGLASLVIAGHSFNVAELLALNSSEATISTPYGELTLTGFSGDLTGGTVQYEYTLNENVDNDSATNANDTDYTDHISVTVIDVDGSQITDSLDVKIVDDASTALDDRGEVDIVADSFTVSGVVANWTSWSNGTNVTTFDGTNAPNGGGLDNDSGKDQIRWGQPASSYSSGYGFIDNDSALNGEFALNQDIILGTFTHYNYPVYSGGAITSASMDVAFSVTDAHGVLTPVTLKLNFDHNETPNTNNPEASKDIIKVGNTNVTFENAGALYTLQVIGFRIPGTNQIVTEIRTGENATNSYELVVRVGPGEGYELPSTSGNVLSNDVSGADVDMTVVGAASGNHVSSGVSGSVGSMIAGLYGNLILLADGSYTYQVTANASSIPNDAIEIFTYTMKDGDGDTSTALLSINVNRVTMADFNANQDHKVGLEDTVVAGNVLDNDGSKNTSVDHFTVGNDATSHVVGSPVSLEQGELTLNSDGSYTFKPADNWNGEVPVITYTTHTGKTSTLAITITPVDDATVTQPDHKSIAEDTIATGNVLANDCDIDSALSVTSFHVEGVNGVYTAGNTMYQLAEGTLVLKANGDYTFDPKDNWSGSLPEITYTTNTGATGTLNIHVEAVADVPNLTINGYTSVAAINFEDARLNGSWDGVVANQIKGLNTIGTWHTSNNSGKVEIGYENIYVSGGSSTNKVMEIEFNNGDKTLYTDIHAQAGRFYELDFDIAARAGSVNSSGLTIKLVPLNAYGVPILAEAITLYDFNPTNANWLRDQKVTLPIDQTGEYRLLFESDDANSYGAILDNLAFKVVDNMGYRGDFIKLSEISTSLNDTDTSETLSLKLKGMPEGSILKDDKGHEVTVGSNGEVDITGWDYSSLQIKTPNHGNFNITVEATATESSNQDSATTSATIPVTVLHPNEYLGRGGVDSFLLTKSNGDNANLNIALNAYYEGTTAVAPVTQQVAVTIDTDLVIHSGNSNDYIDLGISRADNTVYTGSSIPNFNNSTPSQSTLADSAFMKNDVITDHDGVLLQSVQSQIQPITDTVNLGSGNDTVYGGGGNLAAYGGAGNDTLIGGDGNDALRGGADNDYLSGGRGNDVLRGDSGNDVLIGGLGHDILTGGSGEDLFKWVDGDLDGSTDRITDFHLSEKDKIDLSDLFDNPSEQEVTALLDSIKSTVQGDDHSSSFKVEKNDGSSVTIQLDGVSSVELINNLASIIQIKEDLEYLGLEWKTKTVPYLGVEWRTKTVSYWFFGWHTKQVAYLAPVWKEKTIPYAVPVTLSK

SEQ ID NO: 32 - protein, 763 aa, synthetic construct
MSYYHHHHHHDYDIPTTENLYFQGAMGSNINEPSGEAADIISQVADSHAIKYYNAADWQAEDNALPSLAELRDLVINQQKRVLVDFSQISDAEGQAEMQAQFRKAYGVGFANQFIVITEHKGELLFTPFDRTEEIDPALLEAPRTAALLGRSGFASPAPANSETNTLPHVAFYISVNRAISDEECTFNNSWLWKNEKGSRPFCKDANISLIYRVNLERSLQYGIVGSATPDAKIVRISLDDDSTGAGIHLNDQLGYRQFGASYTTLDAYFREWSTDAIAQDYRFVFNASNNKAQILKTFPVDNINASYLGLEWKTKTVPYLGVEWRTKTVSYWFFGWHTKQVAYLAPVWKEKTIPYAVPVTLSKGTTYNTQDYRIERNAKNAQAVSFTWNRQQYATAESLLNRSTDALWVNTYPVDVNRISPLSYASFVPKMDVIYKASATETGSTDFIIDSSVNIRPIYNGAYKHYYVVGAHQSYHGFEDTPRRRITKSASFTVDWDHPVFTGGRPVNLQLASFNNRCIQVDAQGRLTANMCDSQQSAQSFIYDQLGRYVSASNTKLCLDGAALDALQPCNQNLTQRWEWRKGTDELTNVYSGESLGHDKQTGELGLYASSNDAVSLRTITAYTDVFNAQESSPILGYTQGKMNQQRVGQDNRLYVRAGAAIDALGSASDLLVGGNGGSLSSVDLSGVKSITATSGDFQYGGQQLVALTFTYQDGRQQTVGSKAYVTNAHEDRFDLPDAAKITQLKIWADDWLVKGVQFDLN

SEQ ID NO: 33 - protein, 59 aa, synthetic construct
MDYLGLEWKTKTVPYLGVEWRTKTVSYWFFGWHTKQVAYLAPVWKEKTIPYAVPVTLSK

SEQ ID NO: 34 - protein, 89 aa, synthetic construct
MKYLLPTAAAGLLLLAAQPAMAMDYLGLEWKTKTVPYLGVEWRTKTVSYWFFGWHTKQVAYLAPVWKEKTIPYAVPVTLSKLEHHHHHH

SEQ ID NO: 35 - protein, 78 aa, synthetic construct
MGSSHHHHHHSSGLVPRGSHMYLGLEWKTKTVPYLGVEWRTKTVSYWFFGWHTKQVAYLAPVWKEKTIPYAVPVTLSK

SEQ ID NO: 36 - skipped

SEQ ID NO: 37 - protein, 57 aa, synthetic construct
YLGLEWATATVPYLGVEWATATVSYWFFGWATAQVAYLAPVWAEATIPYAVPVTLSK

SEQ ID NO: 38 - protein, 57 aa, synthetic construct
YLGLELKTKTVPLLGVELRTKTVSLWFFGLHTKQVALLAPVLKEKTIPLAVPVTLSK

SEQ ID NO: 39 - protein, 57 aa, synthetic construct
YLGLEWKAKAVPYLGVEWRAKAVSYWFFGWHAKQVAYLAPVWKEKAIPYAVPVTLSK

SEQ ID NO: 40 - protein, 57 aa, synthetic construct
YLGLEAKTKTVPALGVEARTKTVSAWFFGAHTKQVAALAPVAKEKTIPAAVPVTLSK

SEQ ID NO: 41 - protein, 45 aa, synthetic construct
YLGLEWKTKTVPYWRTKTVSYWHTKQVAYWKEKTIPYAVPVTLSK

SEQ ID NO: 42 - protein, 57 aa, synthetic construct
YLGLEWKTKTVPYLGVEWRTKTVSYLGPEWHTKQVAYLAPVWKEKTIPYAVPVTLSK

SEQ ID NO: 43 - protein, 8 aa, synthetic construct
WKTKTVPY

SEQ ID NO: 44 - protein, 24 aa, synthetic construct
LGLEWKTKTVPYLGVEWRTKTVSY

SEQ ID NO: 45 - protein, 11 aa, synthetic construct
VSYWFFGWHTK

SEQ ID NO: 46 - protein, 4 aa, synthetic construct
WFFG

(19) United States
(12) Patent Application Publication
(10) Pub. No.: US 2026/0103629 A1
(43) Pub. Date: Apr. 16, 2026

(54) COMPOSITIONS OF WET ADHESIVES DERIVED FROM VIBRIO CHOLERAE BIOFILM ADHESINS AND METHODS THEREOF

(71) Applicants: YALE UNIVERSITY, New Haven, CT (US); WESLEYAN UNIVERSITY, Middletown, CT (US)

(72) Inventors: Jing YAN, New Haven, CT (US); Richard Aron OLSON, III, Middletown, CT (US)

(21) Appl. No.: 19/112,977

(22) PCT Filed: Sep. 20, 2023
(86) PCT No.: PCT/US2023/074691

§ 371 Date: Mar. 19, 2025

Related U.S. Application Data
(60) Provisional application No. 63/376,414, filed on Sep. 20, 2022.

(51) Int. Cl. C09J 189/00; C07K 14/195

(52) U.S. Cl. CPC C09J 189/00; C07K 14/195

(57) ABSTRACT

Bio-adhesive derived from Vibrio cholerae (Vc) are provided. In some forms, the bio-adhesive includes a sequence of 57 amino acids within a major biofilm adhesin Bap1, and sequence variations thereof, that can be used as bioactive glue to adhere to various surfaces with different chemistries. Methods of producing and using the peptides are also provided.

### CROSS-REFERENCE TO RELATED APPLICATIONS

This application claims the benefit of and priority to U.S. Provisional Application No. 63/376,414 filed Sep. 20, 2022, which is hereby incorporated by reference in its entirety.

### STATEMENT REGARDING FEDERALLY SPONSORED RESEARCH

This invention was made with government support under GM146253 awarded by National Institutes of Health. The government has certain rights in the invention.

### REFERENCE TO SEQUENCE LISTING

The Sequence Listing submitted as a text file named “YU 8510 PCT ST26.xml”, created on Sep. 20, 2023, and having a size of 52,821 bytes is hereby incorporated by reference pursuant to 37 C.F.R. § 1.52(e)(5).

### FIELD OF THE INVENTION

The invention is generally directed to compositions of adhesives that function in wet environments and more specifically to adhesive proteins derived from microbial sources.

### BACKGROUND OF THE INVENTION

Adhesives that function in wet environments are not well-developed, hindering many engineering applications such as those used in ships, underwater vehicles, the fishing industry, etc., often leading to catastrophic failure (Yang, et al., Advanced Functional Materials 30, 1901693 (2020), Narayanan & Joy, Chem. Soc. Rev. 50, 13321-13345 (2021)). Also, bio-adhesives are extensively used in biomedical applications where there is a need to adhere two wet surfaces (adhere two surfaces under water) (Yuen, et al., Chemical Engineering Journal 431, 133372 (2022)).

To search for better adhesive materials that function in an aqueous environment, engineers have extensively studied adhesive proteins from mussels and barnacles, leading to major insights into biological adhesion (Li & Cao, Nanoscale Adv. 1, 4246-4257 (2019), Waite, J. Expt. Biol. 220, 517-530 (2017), Geng, et al., Chem. Res. 55, 1171-1182 (2022)), (Ahn & Waite, Nano-adhesive and surface primer compound and use thereof (2017), Waite, et al., Adhesive materials and methods of making and using the same (2015), Cha, et al., Recombinant mussel adhesive protein fp-131 (2012)). Inspired by the molecular design of mussel foot proteins (mfps), scientists have engineered the key building blocks of mfps into synthetic polymers and purified variations of mfps (Ahn, et al., Nat. Commun. 6, 8663 (2015), Xie, et al., J. Mater. Chem. A 7, 21944-21952 (2019), Fan, et al., Nat Commun 10, 5127 (2019), Maier, et al., Science 6 (2015), Li, et al., Nat. Commun. 11, 3895 (2020), Kim, et al., J. Am. Chem. Soc. 144, 6261-6269 (2022), Horsch, et al., Angew. Chem. Int. Ed. 57, 15728-15732 (2018)). However, mfps are sensitive to environmental conditions including oxygen and pH (Yu, et al., Nature Chemical Biology 7, 588-590 (2011), Yu, et al., Advanced Materials 23, 2362-2366 (2011)), limiting their wide application. Additionally, there is often a need to anchor a protein of interest (such as an enzyme) onto a surface, which demands a short sequence that can be integrated into the protein sequence as an adhesive unit.

An alternative source for such molecules is microbes, such as bacteria. Bacterial biofilms are surface-attached communities of bacterial cells enclosed within an extracellular matrix (Ghannoum, et al., Microbial Biofilms, Washington, DC: ASM Press (2015)). Biofilms not only represent an important lifestyle niche for bacteria in the environment, but also pose a serious threat to human health due to their role in persistent infections and surface fouling of medical devices (Flemming, et al., Nat. Rev. Microbiol. 14, 563-575 (2016), Hall-Stoodley, et al., Nat. Rev. Microbiol. 2, 95-108 (2004)). For example, biofilm-dwelling bacteria can be up to 1000-times more resistant to antibiotics compared to their planktonic counterparts (Allison, et al., Nature 473, 216-220 (2011); Costerton, et al., Science 284, 1318-1322 (1999)). To survive physical forces in natural niches and to colonize hosts, biofilms must adhere firmly to foreign surfaces (Jiang, et al., Proc. Nall. Acad. Sci. USA. (2021)). This is not a trivial task for bacterial cells, which have little control over what surfaces they will encounter. On one hand, to attach to biotic surfaces, biofilm-dwelling cells need to produce specific adhesion molecules to recognize ligands on host surfaces (Lee, et al., Trends in Microbiology). On the other hand, to adhere to diverse abiotic surfaces in the natural environments, biofilms also develop a generic strategy to adapt to various surface chemistries (Berne, et al., Microbiol. 16, 616-627 (2018)). Therefore, biofilm-forming cells need to balance specificity versus generality in terms of the surfaces they recognize to provide robust adhesion in various environments.

It is unclear how bacterial cells achieve this balance, partly due to an insufficient understanding of the biochemical and biophysical mechanisms underlying the collective adhesion of biofilm-dwelling cells, in contrast to the vast knowledge on classical bacterial adhesins that function at the cellular level (Trends Microbiol. 20, 30-39 (2012)). Such a mechanistic understanding is relevant for designing new biofilm removal strategies that target biofilm-surface interactions as an alternative to antibiotic treatments, and more forward thinking, new biofilm-based biomaterials (Huang, et al., Nat. Chem. Biol. 15, 34-41 (2019), Jiang, et al., Proc. Natl. Acad. Sci. USA. (2018)).

Biomaterials based on mussel foot proteins were previously described (US Patent Application Publication Number 2012/0202748 A1), however these biomaterials were difficult to produce, use and manipulate.

In summary, there is a need for adhesives that function effectively in wet environments, to bond both biotic and abiotic surfaces.

Thus, it is an object of the invention to provide molecular compositions that bind to biotic and/or abiotic surfaces in aqueous environments.

It is a further object of the invention to provide methods of combining one or more additional materials with adhesive molecules to form chimeric, adhesive materials that can bind to biotic and/or abiotic surfaces in aqueous environments.

It is a further object of the invention to provide compositions and methods for attaching molecules to aqueous surfaces in a desired and controllable manner.

### SUMMARY OF THE INVENTION

It has been established that a fragment of a protein derived from Vibrio cholerae has adhesive properties in aqueous environments, and that the adhesive properties can be imparted to polypeptides and fusion proteins that incorporate the fragment.

Adhesive polypeptides including or consisting of one or more adhesive domains including an amino acid sequence of SEQ ID NO:1 or 4, or a functional fragment or variant thereof, is provided. Typically, neither the adhesive domain nor the adhesive polypeptide are 100% identical to amino acid sequence of SEQ ID NO:2 or 6 over its entire length. In some forms, the adhesive polypeptide further includes one or more additional heterologous amino acid sequences. The adhesive polypeptide can be a fusion protein.

Adhesive polypeptides complexed to, complexed with, or otherwise associated with one or more additional heterologous molecule(s) are also provided. In some forms, the heterologous molecule is selected from an amino acid, a protein, a nucleic acid, a carbohydrate, a lipid, a metal, a polymer, a cell, a virion, a small molecule, and a mineral, or combinations thereof. In some forms, the heterologous molecule improves a physicochemical property of the polypeptide selected from the group including solubility, adhesion force, cross-linking, and improvement in protein expression, purification, recovery rate, and biodegradability of the adhesive protein.

Adhesive polypeptides including at least 70% and less than 100% sequence identity to SEQ ID NO:1 or 4, or functional fragment thereof, are also described. Typically, the polypeptide adheres to a surface under aqueous conditions. In some forms, the variant includes at least 70% sequence identity of SEQ ID NO:1 or 4, or a functional fragment thereof.

In some forms, the polypeptide further includes a heterologous amino acid sequence and/or a heterologous molecule. In some forms, the variant or fragment is between 25 and 70 amino acids inclusive, or any subrange or specific integer therebetween. In some forms, the heterologous amino acid sequence and/or heterologous molecule includes one or more purification tags.

In some forms, the polypeptide adheres to an abiotic surface in an aqueous environment. Exemplary abiotic surfaces include a material selected from metal, stone, plastic, glass, silica, concrete, paint, carbon, rubber, ceramic, Polytetrafluoroethylene (PTFE) and polymer fabric, or combinations thereof.

Polypeptides having an amino acid sequence that is at least 70 to 99% identical to SEQ ID NO:1 or 4, wherein one or more residues are mutated compared to SEQ ID NO:1 or 4, and wherein the polypeptide does not adhere to a surface are also described. In some forms, the mutation(s) includes one or more Lysine to Alanine.

Nucleic acids encoding the polypeptide including an amino acid sequence of SEQ ID NO:1 or 4, or a functional fragment or variant thereof are also provided. In some forms, the nucleic acid is RNA or DNA. In some forms, the nucleic acid includes a vector, such as an expression vector.

Cells including or expressing the adhesive polypeptides, or a nucleic acid encoding or expressing the polypeptide are also provided. In some forms, the polypeptide is heterologous to the cell. In some forms, the cell is selected from the group including a bacterium, a protozoan, a plant cell, an insect cell, a mammalian cell, a yeast cell and a fungal cell.

Compositions including the adhesive polypeptide are also provided. Typically, the polypeptide forms an adhesive layer within or on the composition.

In some forms, the adhesive layer is in contact with a surface, and wherein the adhesive layer adheres the composition to the surface. In some forms, the surface includes a material selected from the group including metal, stone, plastic, glass, silica, concrete, paint, carbon, rubber, ceramic, and polymers, or combinations thereof. In some forms, the adhesive polypeptide includes between 0.1% and 50% by weight of the total composition.

An adhesive glue, including (i) the adhesive polypeptide including an amino acid sequence of SEQ ID NO:1 or 4, or a functional fragment or variant thereof; and (ii) an excipient is also described. Typically, the adhesive polypeptide is suspended or mixed within the excipient. In some forms, the adhesive polypeptide includes between 0.1% and 99.9% by weight of the glue. In some forms, the adhesive polypeptide includes between 1% and 50% by weight of the glue. In some forms, the glue is in the form of a liquid, a gel, an emulsion, a cream, an aerosol, a powder, or a foam. In some forms, the excipient is not an aqueous solution. In some forms, the excipient is selected from the groups including a surfactant, an oxidant, and a filler. In some forms, the filler is selected from the group including collagen, hyaluronic acid, condroitan sulfate, elastine, laminin, caseine, hydroxyapatite, albumin, fibronectin, and hybrin.

A coating material including the glue is also described. A container including the glue or coating material is also provided. Typically, the container includes means for extrusion of the glue or coating material. In some forms, the glue or coating material does not adhere to the inside of the container.

A method of adhering two or more compositions together, including (i) contacting a first composition with an adhesive polypeptide, or with a glue or coating containing the polypeptide, to form a first adhesive composition including an adhesive layer; and (ii) contacting a second composition with the first adhesive composition, wherein the contacting includes interaction between the second composition and the adhesive layer, sufficient for the first adhesive composition and second composition to adhere together.

In some forms, wherein the contacting in step (i), or step (ii), or in both steps (i) and (ii) occurs in an aqueous environment. In some forms, the adhesive layer includes the adhesive polypeptide, or the glue in an amount between about 0.01 μg/cm² and about 100 μg/cm², inclusive. In some forms, the aqueous environment is underwater. In some forms, the underwater environment is in an ocean or sea, or a lake, river, loch, reservoir or other body of water.

### BRIEF DESCRIPTION OF THE DRAWINGS

FIGS. 1A-1C are graphic representations of the nucleic acid expression constructs (FIG. 1A), and structural representation using a ribbon diagram (FIG. 1B) of the wild-type Bap1 and RbmC proteins, respectively, showing the constituent domains in each protein labelled beta-gamma-crystallin, beta-propeller (blades 1-5), beta-prism B/Cl, beta-propeller (blades 6-8), and beta-prism C2, respectively. The position of the 57-aa loop is indicated within the beat-prism B domain; the position of the velcro closure motif is indicated within the beta-propellor; and a representation of the beta-propeller structure is shown. FIG. 1C is a schematic representation of the domains and the corresponding representation of Bap1 (Left) and RbmC (Right) mutants used in the study. The relative positions of the K574A and D539A mutations is depicted.

FIGS. 2A-2G show that Bap1 and RbmC mutants in the β-prism show adhesion defects. FIG. 2A shows sectional confocal images of biofilm from ΔrbmC Δ57-aa; FIG. 2B shows sectional confocal images of biofilm from Δbap1 Δβ-prismC1; FIG. 2C shows sectional confocal images of biofilm from Δbap1 Δβ-prismC2; FIG. 2D shows sectional confocal images of biofilm from Δbap1 Δβ-prismC1C2; FIGS. 2E-2F are graphs of data from a Biofilm adhesion assay for Bap1 mutants in the +ΔrbmC background (FIG. 2E) and RbmC mutants in the Δbap1 background (FIG. 2F), respectively, showing fraction of biomass remaining after washing over BSA (mg/ml) in each graph. FIG. 2G shows the 57 amino acid sequence: YLGLEWKTKTVPYLGVEWRTKTVSYWFFGWHTKQVAYLAPVWKEKT IPYAVPVTLSK (SEQ ID NO:1), annotated with dashes depicting aromatic residues, and dots depicting positively charged residues.

FIG. 3A is a ribbon diagram depicting the structural comparison/overlay of ribbon diagrams representing β-prismC1 and C2, with a tri-mannose molecule depicted in ball and stick format, in the glycan binding pocket. Arrows indicate location of the β-prismC1 lysine (K574) and a nearby aromatic tyrosine (Y576). FIG. 3B is a Biofilm adhesion assay of strains with the indicated point mutations in β-prismC1, showing the fraction of biomass remaining after washing over BSA (mg/ml) for each of Δbap1Δβ-prismC2 ( [symbol] ); Δbap1Δβ-prismC2 RbmC^{K574A}( [symbol] ); ΔbaplA-prismC2 RbmC^{KYY574AYA} ( [symbol] ); Δbap1Δβ-prismC2 RbmC^{KYY574LYN} ( [symbol] ) All data are depicted as the mean±SD from at least 3 biological replicates.

FIGS. 4A-4D are schematic representations of biofilms formed by each of Δbap1,rbmc (FIG. 4A), or ΔrbmC,bap1 (FIG. 4B), Δbap1,ΔprismC2K574A (FIG. 4C), and Δbap1,ΔprismC2K539A (FIG. 4D), respectively, depicting each protein's interaction with an asialofetuin-coated surface.

FIGS. 5A-5D are annotated images of protein gels, showing the binding to purified VPS of purified Bap1's β-propeller with prism (FIG. 5A), Bap1's β-propeller alone (FIG. 5B), prism alone (FIG. 5C), and GFP alone (control) (FIG. 5D), respectively. Arrows with brackets indicate protein-VPS complex, black arrows indicate unbound proteins.

FIGS. 6A-6E show that disruption of Velcro closure in β-propeller produces a secreted but deficient protein. FIG. 6A is a histogram showing the fraction of biomass remaining after washing for each of ΔrbmC; ΔrbmC,ΔVelcro; and ΔrbmC,Δbap1. FIG. 6B is an image of a western blot stained for V. cholerae strains expressing 3×FLAG-tagged WT or Bap1ΔVelcro protein (* denotes strain lacking HapA and IvaP) FIG. 6C is a schematic representation of a protocol for the release of biofilm matrix proteins into the supernatant upon deletion of vpsL. FIG. 6D is an image of a western blot stained for 3×FLAG-tagged WT or Bap1ΔVelcro protein with or without vpsL, showing each of lanes loaded with WT(S); WT(P); ΔrbmC; ΔVelcro(S); ΔVelcro(P); ΔVelcro*(S); and ΔVelcro*(P), where (S) depicts supernatant and (P) depicts pellet, respectively. FIG. 6E is a graph of Dynamic Light scattering, showing frequency (%) over size (nm) for each of the solutions of VPS (left hand side curve) and VPS+Bap1 (right hand curve), respectively.

FIGS. 7A-7B are schematic representations of the distinct adhesion functions of RbmC and Bap1 proteins. FIG. 7A depicts a working model of how Bap1 enables V. cholerae biofilms to adhere to abiotic foreign surfaces. FIG. 7B depicts a working model of how RbmC enables V. cholerae biofilms to adhere to biotic foreign surfaces. VPS, Bap1 and RbmC are indicated. FIG. 7C is a phylogenetic analysis of Bap1 and RbmC homologs in Vibrio species. Scale bar: nucleotide substitutions per site.

FIGS. 8A-8B show the amino acid sequence of the 57-aa loop nested in Vibrio cholerae adhesin that is responsible for biofilm adhesion to various abiotic surfaces. FIG. 8A depicts the sequence, annotated with positive residues (indicated by dots) and aromatic residues (underlined), respectively. FIG. 8B depicts the sequence, aromatic residues (underlined), respectively boxes indicating the repeating motif. FIG. 8C depicts the sequence logo corresponding to this sequence, showing the most conserved amino acids in the repeating motif.

FIGS. 9A-9F show a microbeads adsorption assay for quantifying adhesive properties of peptides. FIG. 9A is a graph of the Langmuir adsorption curve of FITC-labeled 57-aa peptide with silica beads (●), and FITC (▪) as a control, showing I_{ring}-Iₛₒₗᵤₜᵢₒₙ (a.u.) over [solute](μM). Inset is a representative image of adsorption intensity quantification using MATLAB. FIGS. 9B-9C are graphs of Flow cytometry-based quantification of the microbeads adsorption assay, showing Counts over FITC-A for each of 1.5 μM FITC (FIG. 9B), and 1.5 μM FITC-labeled 57-aa peptide (FIG. 9C) adsorbed on 5 μm silica beads, respectively. FIG. 9D shows sequences with different variations of the original 57-aa peptide sequence tested for adhesive properties. FIG. 9E and FIG. 9F are a pair of plots demonstrating the effect of sequence variation on adhesion to abiotic surfaces. Biofilms from V. cholerae strains with different sequences in the insertion loop were subjected to the adhesion assay on glass substrate.

FIGS. 10A-10F are graphs depicting adsorption of the 57-aa peptide on surfaces with varying chemistry. FIG. 10A is a schematic of surface modification of silica beads with organo-silanes. FIG. 10B, is a graph of Zeta potential measurements of different microbeads, showing Zeta potential (mV) for each of carboxylate lates, sulfate latex, silica, propyl silica, and amine silica at each of 10 mM NaCl pH 7 and pH 3, respectively. Silica beads were obtained using the method showed in a starting from the commercially purchased, pristine silica beads; latex beads were commercially purchased that have preexisting surface modifications as indicated. FIGS. 10C-10F are graphs of adsorption curves of FITC-labeled 57-aa peptide (●) and FITC (▪) as control, showing I_{ring}-Iₛₒₗᵤₜᵢₒₙ (a.u.) over [solute](μM), obtained with amine silica beads (—CH₂CH₂CH₂NH₂, (FIG. 10C); propyl silica beads (—CH2CH2CH3; (FIG. 10D); carboxylate modified latex beads (FIG. 10E); and sulfate modified latex beads (FIG. 10F), respectively.

FIGS. 11A-11B are graphs quantifying adhesive strength using atomic force microscopy (AFM). FIG. 11A is a graph of the force curve of separating two surfaces adhered to each other via the 57-aa peptide (solution concentration=L.5 μM), showing Force over ZSnsr (distance) for each of probe approach, and probe retract, respectively. Black double arrow corresponds to the detachment force (maximum adhesive force). Inset is depicted a schematic of the AFM experiment setup using silica beads on a Mica surface. FIG. 11B is a histogram graph showing distribution of the detachment force (N=95) in nN, with the curve corresponding to a gaussian fitting of the data, yielding a mean value of 2.82 nN.

FIGS. 12A-12C demonstrate adhesion of the 57aa peptide to lipid-coated surfaces. FIG. 12A is a series of images depicting the results of adsorption assays for quantifying adhesive properties of the 57aa peptide to lipids. Top: a representative image of 5 μm silica beads coated with lipids, labeled with RhPE (Top, Left) for lipids and the FITC-labeled 57aa peptide adsorbed on lipid layer (Top, Right). Bottom: the same experiment performed with a FITC control. FIG. 12B is a plot demonstrating excess fluorescence signal on the surface of the beads coated with lipids compared to the solution signal. FIG. 12C is graph showing excess intensity of FITC-labeled 57-aa peptide on lipid-coated beads as a function of peptide concentration in the solution, for different lipid compositions.

FIGS. 13A-13B demonstrate adhesion of the 57-aa peptide sequence to tissues. FIG. 13A shows confocal images of human jejunum tissue slices stained with 1 μM FITC-labeled 57aa peptide. FIG. 13B shows representative maximum projection images of mouse enteroid monolayers stained with DAPI, an F-actin probe conjugated to Alexa Fluor™ 647 dye, and 1 μM labeled 57aa peptide. Shown on the left column are the overlay images of all three fluorescent channels and on the right column are the signals in the 488 nm channel.

FIG. 14 is a demonstration of the flocculation ability of the 57-aa peptide. Shown are large aggregates formed by the 57aa peptide (3 μM) and 200 nm fluorescent polystyrene particles (0.0002%).

FIGS. 15A-15C shows gels images of SDS-PAGE demonstrating whole cell expression of the 57-aa peptide sequence (+His) from E. coli cells stained by Coomassie (FIG. 15A) or using an anti-his antibody Western blot (FIG. 15B) expressed at three temperatures (° C.). Ladder size markers are in kDa. (FIG. 15C) 6M Urea-solubilized 57-aa (+His). Arrow denotes location of 57-aa.

### DETAILED DESCRIPTION OF THE INVENTION

### I. Definitions

As used herein, the term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “functional fragment” as used herein is a fragment of a full-length protein retaining one or more function properties of the full-length protein.

As used herein, the term “variant” refers to a polypeptide or polynucleotide that differs from a reference polypeptide or polynucleotide but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological forms. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Forms of this disclosure thus contemplate functional or biological equivalents of a polypeptide as set forth above. In particular, forms of the polypeptides can include variants having about 50%, 60%, 70%, 80%, 90%, and 95% sequence identity to the polypeptide of interest.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will assist the linked protein to be localized at the specific organelle.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

### II. Compositions

### A. Adhesive Polypeptides

It has been established that a 57 amino acid fragment of the biofilm derived from Vibrio cholerae (Vc) is functional as an adhesive peptide in isolation. Adhesive polypeptides and compositions thereof for forming adhesion to surfaces are provided. The adhesive polypeptides include or consist of an adhesive domain(s), and can further additional amino acids sequence(s) that are optionally heterologous the adhesive domain(s). Thus, for example, in some forms, the adhesive polypeptide consists of an adhesive domain. In other forms, the adhesive polypeptide is a fusion protein including an adhesive domain(s) and a heterologous sequence.

Compositions including and encoding polypeptides including an adhesive domain are also described.

The compositions can include adhesive polypeptides, as well as nucleic acids, vectors and cells encoding and/or expressing the adhesive polypeptides. Glues and coatings including the adhesive polypeptides are also described. Compositions including or incorporating an adhesive component are also provided. In some forms, the compositions include or are bound together by a glue or coating including the adhesive polypeptides.

1. Vibrio cholerae Biofilm Adhesins

Adhesive polypeptides derived from Vibrio cholerae, and variants thereof are described. Growth of the cholera bacterium Vibrio cholerae in a biofilm community contributes to both its pathogenicity and survival in aquatic environmental niches. The major components of V. cholerae biofilms include Vibrio polysaccharide (VPS) and the extracellular matrix proteins RbmA, RbmC, and Bapt. Proteins and polynucleotides of Vibrio cholerae are described in United States Patent Application Publication No. 2011/0003734, the contents of which are herein incorporated in their entirety. Analysis of the Vibrio cholerae biofilm is described in Kaus, et al., J. Biol. Chem. (2019) 294(40) 14499-14511, the contents of which are herein incorporated in their entirety.

The major components of the V. cholerae biofilm matrix are proteins RbmA, RbmC, and Bap1. Vibrio polysaccharide is formed by repeating units of an acetylated tetrasaccharide unique to V. cholerae, whose synthesis and export are carried out by the products of the vps I and vps I gene clusters. RbmA and RbmC are two of six proteins encoded by the Rugosity and Biofilm Modulators (rbm) gene cluster. Bap1 (Biofilm-Associated Protein 1), which shares substantial sequence identity with RbmC, is encoded by a single gene (VC1888) that is located far from the vps and rbm gene clusters. Synchronized up-regulation of vps I, vps II, and rbm gene clusters, as well as bap1, has been shown to occur during the biofilm production life stage of V. cholerae. The high-resolution structure of RbmA uncovered a composition of tandem fibronectin type III domains and provided substantial insight into its contribution to the V. cholerae biofilm matrix. Less is understood about the structure and molecular mechanisms underlying the adhesive and scaffolding roles played by Bap1 and RbmC.

Insights into the function of the biofilm matrix components have come from knockout mutagenesis studies of the biofilm matrix proteins (RbmA, RbmC, and Bap1) and microscopy utilizing fluorescently-labeled components of the biofilm. Investigation into V. cholerae biofilm formation and architecture
often utilizes so-called rugose strains, which exhibit increased biofilm production, wrinkled colony morphologies, and the formation of a floating structure called a pellicle (Kaus, et al., J. Biol. Chem. (2019) 294(40) 14499-14511; Beyhan and Yildiz, Mol Microbiol, 2007 February; 63(4):995-1007, and references therein).
   a. Bap1 (Biofilm-Associated Protein 1)

In some forms the adhesive polypeptide includes all or part of the adhesin Biofilm-Associated Protein 1 (Bap1) from V. cholerae.

In the aquatic niche, the ability of Bap1 to bind anionic polysaccharides or abiotic surfaces provides a survival advantage by promoting attachment to a multitude of substrates, including extracellular polysaccharides found on phyto- and zooplankton or macroflora such as macroalgae. In addition, the increased elasticity provided to the biofilm matrix by Bap1 confers increased tensile strength that aids in survival in environments where dynamic movement (such as ocean currents) is abundant. Bap1 plays an important role in attaching V. cholerae biofilms to both biotic and abiotic surfaces in the aquatic niche.

The crystal structure of Bapt at 1.9 A° resolution revealed a two-domain assembly made up of an eight-bladed beta-propeller domains interrupted by a beta-prism domain. The structure also revealed metal-binding sites within canonical calcium blade motifs, which appear to have structural rather than functional roles. Details of the structure of the Bap1 protein, without the 57 amino acid polypeptide of SEQ ID NO:1 from Vibrio cholerae is described in Kaus, et al., J. Biol. Chem. (2019) 294(40) 14499-14511, the contents of which are herein incorporated in their entirety.

Compositions including Bap1 proteins, and functional fragments thereof are provided for use as adhesive moieties. An exemplary amino acid sequence for the Bap1 protein of V. cholerae is set forth in the NCBI protein databank under accession number WP_001881639.1, having 691 amino acids and a sequence of:

(SEQ ID NO: 2)
MKQTKTLTAISVLALSHLMTQSTAFASSSSDIQTKLKWSWSTSVF
HPESNQVMAAPIVVQLNDDNGDGKIDEKDVADIIVVTFEGNKYAN
GGYIRALSGVDGSELWSYSNGGVIADARYAPAAADLDGDGLIEIV
STSALTPYINILDHQGNIKKQLLKSASGWRSVGDIALADINGDGN
IEILAADGVYSYESGLLFSHDWAPSSIAFDSNGDGQREVFANGTL
YONNGAYLWQYQANDTVWFSSVANLDGDDKPELVVSVPASLSTPE
NSEIAVLEHDGSVKWRVNNLSNPGGSVQAVSSFLGKPSSSATTVD
AQSAVYGYTDWAHQQRVLAENHQLAIRSGAVVDAIGANSQNMIGG
SGGSLSTIDTSKVRAIDVTYGKNKYTWKYGVLEMSFTLDNGAKVT
VGSKDSAFTYLGLEWKTKTVPYLGVEWRTKTVSYWFFGWHTKQVA
YLAPVWKEKTIPYAVPVTLSKSTTVRYDIPQGSQLLGMNVWSKEK
HLFKHKQQVNAVQFLVGKVTADQSHMGIVYAGYYAVDMYDAQGNK
VWSVANDDLNSGKIGVSAYDFTGDGIDEVLVQDRLRMRILDGQTG
RVMGIIANSSGTLWEYPVVADLEGNNNASLIMVANDYDRESQVNH
GVFVYESANPSKPWRNATRIWNQYAFNFSDINANGTIPTNAQPSW
LTHNSFRSATIRVPLK.

The position of amino acids corresponding to the 57 amino acid loop of SEQ ID NO:1 are underlined. The position of amino acids corresponding to a Beta-prism domain are in italics. The position of the amino acids corresponding to the signal sequence are in bold text.

An exemplary amino acid sequence of the beta-prism domain including the 57 amino acid loop is:

(SEQ ID NO: 3)
DAQSAVYGYTDWAHQQRVLAENHQLAIRSGAVVDAIGANSQNMIG
GSGGSLSTIDTSKVRAIDVTYGKNKYTWKYGVLEMSFTLDNGAKV
TVGSKDSAFTYLGLEWKTKTVPYLGVEWRTKTVSYWFFGWHTKQV
AYLAPVWKEKTIPYAVPVTLSKSTTVRYDIPQGSQLLGMNVWSKE
KHLFKHKQQVNAVQFLVGKV.

An exemplary amino acid sequence of the 57 amino acid loop is:

(SEQ ID NO: 1)
YLGLEWKTKTVPYLGVEWRTKTVSYWFFGWHTKQVAYLAPVWKEK
TIPYAVPVTLSK.

In some forms, the Bap1 has an amino acid sequence of SEQ ID NO:2. In some forms, the Bap1 protein is a functional mutant, variant, or fragment of SEQ ID NO:2. For example in some forms, the Bap1 protein has an amino acids sequence that is less than 100% identical to SEQ ID NO:2. For example, in some forms, the Bap1 protein has an amino acid sequence that is at least 70% up to 99.9%, e.g., 75%, 80%, 85%, 90%, or 95% identical to SEQ ID NO:2, or to a fragment thereof.

In some forms, the variant of SEQ ID NO:2 is a homolog of Bap1 from another organism. Homologs in other Vibrio species indicate that the adhesion biochemistry revealed herein may be conserved in those species. Among them, V. coralliilyticus is a coral pathogen that causes coral bleaching (de O Santos, et al., The ISME Journal 5, 1471-1483 (2011)), V. anguillarum is a fish pathogen (Frans, et al., Journal of Fish Diseases 34, 643-661 (2011)), and V. tubiashii infects mollusks (Hasegawa, et al., Appl Environ Microbiol 74, 4101-4110 (2008)). Thus, in some forms, a variant of the Bapt protein is the homolog of SEQ ID NO:2 in another species such as V. anguillarum or a functional mutant, variant, or fragment thereof.

In some forms, the variant of the Bap1 protein is the Bapt protein from V. anguillarum having NCBI GenBank accession number WP064625571.1, and the amino acid sequence:

(SEQ ID NO: 5)
MTSKFSLCAVGLLSISSIAVSTIATASNPSEINTQLKWSWESSSF
KPESNQVMAAPVIAQLNDDNGDGKIDENDIADIIVVTFENNKYTQ
GGLVRALSGIDGSELWSYDNGGIIADARYSPAVADLDGNGVVDIV
ITSASSPYITILDNEGNIKKQILKHVTGGRSVGTISISDLNNDGS
IEIISADGVYNYDTGLLFSLEWAPSSISFDADGDGVQEIFSNGAL
YKSDGSFTWQYQANDTVWFSSVANLDSDNKPEIVVSVPATKATAQ
NSVFAVLEHDGSVKWEVNNLENPGGGVQAISNFLGNTATSSTNEI
AKSPVYGYTHLHHSHPVKIADDNQLKIRSGDLIDAIGSTASNMVG
GQGGSLHTIDASKVRSVDVTYGKYKTWWTYGVLEMEFTLNDGSKI
TLGSKDSAFKYPALEWRTKEVPYLGLEWRTKQVSYWFFGWHTKTV
SYLAPVWKTKTIPYAVPVMKSKATTERYTVPSNTQLVGLNVWSKP
KPIFTFKKHVNAVQFVVGESINDSYLNTGIVYAGYHAVDMYNAQG
SKVWSVANDDYNSGKIGVSAYDFTGDGIDEVIVQDLLRVRILDGR
TGAVLATIANSSNILWEYPVVADLEGNNNASLIVVANDYAKESAI
NHGVYVYESADADKPWKNATRIWNQHSFHFSNINQDGSVPTNAQP
SWLTHNTYRSSTIK.

An exemplary amino acid sequence of a beta-prism domain from V. anguillarum corresponding to the beta-prism domain of V. cholerae is:

(SEQ ID NO: 6)
IAKSPVYGYTHLHHSHPVKIADDNQLKIRSGDLIDAIGSTASNMV
GGQGGSLHTIDASKVRSVDVTYGKYKTWWTYGVLEMEFTLNDGSK
ITLGSKDSAFKYPALEWRTKEVPYLGLEWRTKQVSYWFFGWHTKT
VSYLAPVWKTKTIPYAVPVMKSKATTERYTVPSNTQLVGLNVWSK
PKPIFTFKKHVNAVQFVVGES.

An exemplary amino acid sequence from V. anguillarum corresponding to the 57 amino acid loop of V. cholerae is:

(SEQ ID NO: 4)
YPALEWRTKEVPYLGLEWRTKQVSYWFFGWHTKTVSYLAPVWKTK
TIPYAVPVMKSK.

In some forms, the Bap1 has an amino acid sequence of SEQ ID NO:6. In some forms, the Bap1 protein is a functional mutant, variant, or fragment of SEQ ID NO:6. For example in some forms, the Bap1 protein has an amino acids sequence that is less than 100% identical to SEQ ID NO:6. For example, in some forms, the Bap1 protein has an amino acid sequence that is at least 70% up to 99.9%, e.g., 75%, 80%, 85%, 90%, or 95% identical to SEQ ID NO:6, or to a fragment thereof.

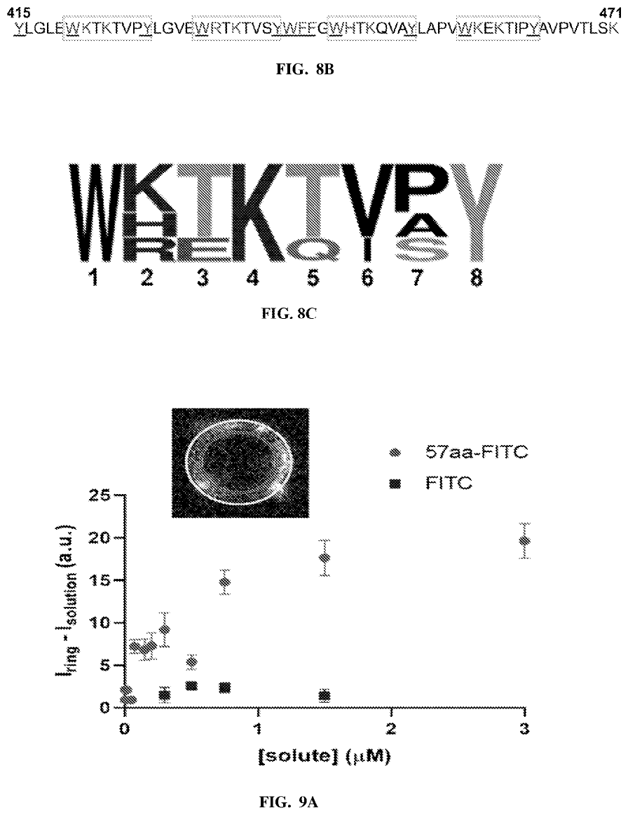

Additionally, the Examples below interrogate the domains and even specific amino acid residues that are important for the adhesive activity of Bap1. Thus, all of the variant sequences (e.g., substitutions, deletions, insertions, etc.) and domain structures (e.g., domain deletions and/or reorganizations) described therein are also expressly incorporated and can be utilized in the construction of the provided adhesive domains and polypeptides. For example, residues and domains found to be important for the adhesion may be expressly maintained or substituted with conservative mutations, etc., and/or residues found to be unimportant for adhesion can be mutated with conservative or non-conservative substitutions, or deleted, etc. See, for example, FIGS. 8A-8C, which provides the sequences of repeat domains of SEQ ID NO:1 (boxed in FIG. 8B) as well as positively charged and aromatic residues believed to be important for adhesion.

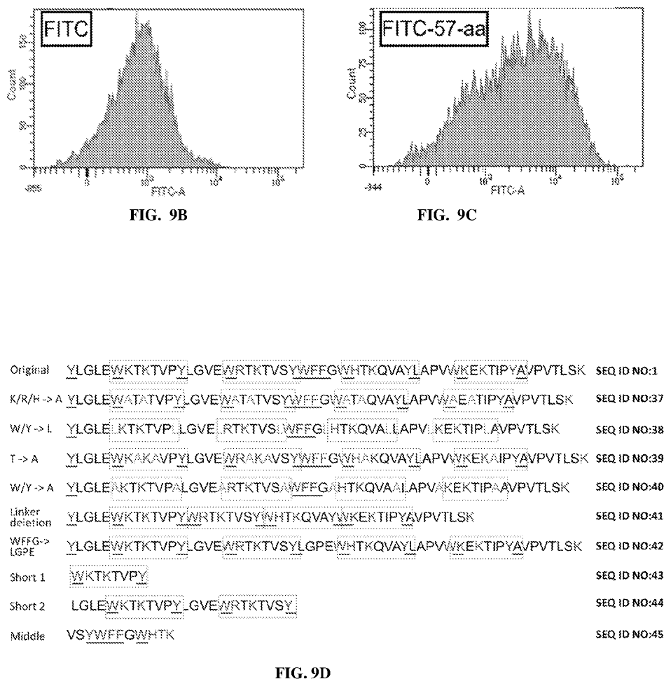

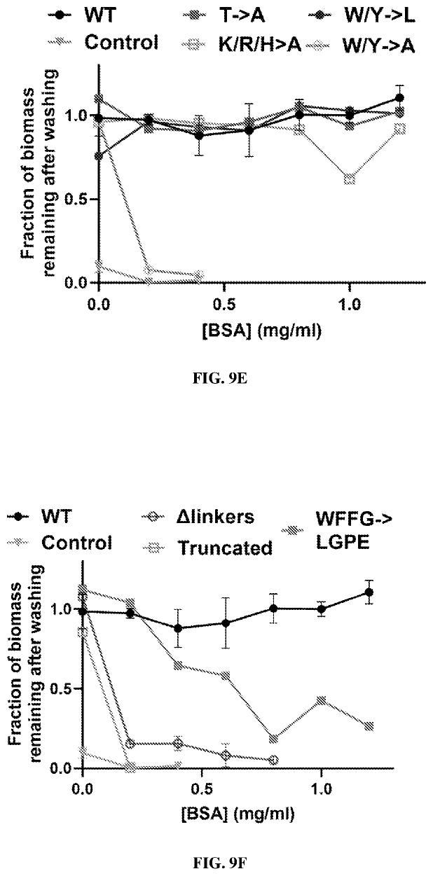

A list of tested sequences is shown in FIG. 9D. Data shown in FIG. 9E and FIG. 9F indicate that the middle sequence WFFG (SEQ ID NO:46), flanked by the repeating motifs, plays a key role in adhesion to various surfaces, while the repeating motif at the periphery plays accessory roles. As shown in the data (FIGS. 9E-9F), removing aromatic residues such as tryptophane and tyrosine in the repeating motif in the periphery leads to a loss in biofilm adhesion to glass substrate. Meanwhile, removing the aromatic central region of the sequence decreases, but does not eliminate glass adhesion. Finally, removing the flexible linkers between the repeating motifs abolishes adhesion to glass, implicating that certain flexibility is important to allow the repeating units to simultaneously contact the glass substrate.

Preferred embodiments include or otherwise retain the WFFG (SEQ ID NO:46) motif, or at least aromatic residues such as tryptophane and tyrosine therein, the flanking repeating motifs, flexible linkers between the repeating motifs, or a combination thereof. In some embodiments, aromatic central region is mutated or removed.

Thus, in some forms, one, two, three, four or more of the repeat domains are present and/or unmutated and/or some or all mutations are conservative; some or all of the positively charged residues are present and/or unmutated and/or some or all mutations are conservative; some or all of the aromatic residues are present and/or unmutated and/or some or all mutations are conservative are present and/or unmutated and/or some or all mutations are conservative; or a combination thereof.

b. Adhesive Domain

It has been established that a sequence of 57 amino acids (57-aa) from the major biofilm adhesin Bap1, and sequence variations or fragments of it can be used as bioactive glues to adhere to various surfaces with different chemistry.

As set forth in the Examples, below, it has been determined that the 57 amino acids having the sequence: YLGLEWKTKTVPYLGVEWRTKTVSYWFFGWHTKQVAYLAPVWKEKTIPYAVPVTLSK (SEQ ID NO:1) mediate adhesion of the Bapt protein of V. cholerae adhesin, and that the isolated 57 amino acid polypeptide of SEQ ID NO:1 has adhesive properties. The adhesive properties facilitate adhesion under aqueous conditions, such as underwater. The adhesive properties include adhesion to abiotic surfaces, such as glass, metals, concrete, ceramics, polymers, fabrics, paints, and combinations thereof. In some forms, the adhesive properties facilitate adhesion to biotic surfaces, such as cells and/or cellular components, including proteins, polysaccharides, lipids, and combinations thereof. Therefore, adhesive domains for adhesion to abiotic and/or biotic surfaces are provided. In some forms, the adhesive domains adhere to the surfaces in an aqueous environment. In some forms, the adhesive domains adhere to abiotic and/or biotic surfaces underwater, such as seawater or freshwater. An exemplary homologue of SEQ ID NO:1 is provided as SEQ ID NO:4.

Therefore, adhesive polypeptides including or having an adhesive domain are described. In some forms the adhesive domain is or includes the amino acid sequence of SEQ ID NO:1 or 4. In some forms, the adhesive domain has an amino acids sequence of SEQ ID NO:1 or 4. In some forms, the adhesive polypeptide is a functional mutant, variant, or fragment of SEQ ID NO:1 or 4. For example in some forms, the adhesive domain has an amino acids sequence that is less than 100% identical to SEQ ID NO:1 or 4. In some forms, the adhesive domain is or includes a sequence homologous to SEQ ID NO:1. For example, in some forms, the adhesive domain includes the sequence from V. anguillarum (SEQ ID NO:4), homologous to SEQ ID NO:1.

In some forms, the adhesive domain has an amino acid sequence that is at least 75% up to 99.9%, e.g., 75%, 80%, 85%, 90%, 91%, 92%, 93%, 94%, 95%, 96%, 97%, 98%, or 99% identical to SEQ ID NO:1 or 4, or a homologous sequence thereto.

In some forms, the adhesive domain polypeptide includes additional amino acid residues from full-length Bap1 polypeptide, but typically does not include the entire full-length protein of SEQ ID NO:2 or 6. In some forms, the additional amino acid residues are continuous with the amino acids of SEQ ID NO:1 or 4 as it sits within SEQ ID NO:2 or 6, respectively. Thus, in some forms, the adhesive domain is a fragment of SEQ ID NO:2 or 6 including SEQ ID NO:1 or 4 or a fragment thereof, or the respective variants thereof, as well as additional amino acids outside of SEQ ID NO:1 or 4). Examples are provided as SEQ ID NO:3 and 5. Fragments can be any specific number of amino acids between about 10 amino acids up to one amino acid less than full-length protein, or any specific integer or range of integers therebetween. Exemplary fragments include 50, 51, 52, 53, 54, 55, 56, 58, 59, 60, 61, 63, 64, 65, 66, 67, 68, 69, 70, 71, 72, 73, 74, 75, 100, 125, 150, 175, 200, 225, 250, 275, 300, 325, 350, 375, 400, 425, 450, 475, 500, 525, 550, 575, 600, 625, 650, or 675 amino acids of SEQ ID NO:2 or 6, or a variant thereof such as a homolog thereof. Variants of fragments are also provided can be e.g., least 70% up to 99.9% identity e.g., 75%, 80%, 85%, 90%, or 95% identical to the wildtype fragment. In preferred forms, the fragment of SEQ ID NO:2 or 6, includes the underlined portion thereof alone or in combination with italics portion (i.e., SEQ ID NO:3 or 5), or its variant or the corresponding sequence in a homolog thereof.

In other forms, the additional amino acids are from a segment or segments of the protein discontinuous with SEQ ID NO:1 or 4 in SEQ ID NO:2 or 6, e.g., intervening sequence(s) are absent or deleted. For example, the experiments below show that a Bap1 construct in which the β-prismB was deleted and the 57-aa directly attached to the β-propeller exhibits fully functional adhesion. Thus, in some forms, the adhesive domain includes SEQ ID NO:1 or SEQ ID NO:4, or a variant or homologous sequence thereto directly attached to a β-propeller, e.g., the β-propeller of SEQ ID NO:2 or 6 or variant thereof, or homologous sequence thereto.

Additional results show that the β-prismB cap together with the 57-aa loop may form a continuous, positively charged surface enabling Bap1 to function as the predominant adhesin interacting with abiotic surfaces. Thus, in some forms, in addition to SEQ ID NO:1 or 4, or a variant or homologous sequence thereto, the adhesive domain further includes the β-prismB cap.

In some forms, the 57 aa loop having a sequence of SEQ ID NO:1 or 4, or a fragment or variant thereof is expressed as a fusion peptide as part of a much larger polypeptide, for example, of up to about 1,000 amino acids, or up to about 2,000 amino acids, or up to about 3,000 amino acids, inclusive. Overall, the probability of successfully expressing a soluble protein decreases considerably at molecular weights above ˜60 kDa. See, e.g., “Protein production and purification,” Nat Methods, 5(2): 135-146 (2008). doi: 10.1038/nmeth.f.202, which is specifically incorporated by references herein in its entirety. Thus, in some forms, the adhesive domain sequence is no more than 100, 90, 80, 70, or 60, amino acids of full-length Bap1 or a variant thereof, or any other specific integer between 100 and 50. For example, in some forms, the length of the fragment or variant sequence thereof from full-length Bap1 or variant that forms part or all of the adhesive domain is between about 40 and 100 amino acids of SEQ ID NO:2 or 6, including all or part of SEQ ID NO:1 or 4 or 3 or 5, or the respective variants thereof, or a subrange thereof or any specific integer number therebetween. In some forms, the entire adhesive domain, including any optional heterologous sequence(s), is no more than 100, 90, 80, 70, 60, 50, or 40 amino acids, or any other specific integer between 100 and 40.

Preferred variants of any of SEQ ID NO:1-6 may avoid mutations believed to be important for adhesion. As introduced above, FIGS. 8A-8C illustrates residues of SEQ ID NO:1 believed to be important for adhesion (i.e., repeat domains and particularly positively charged and aromatic residue). Corresponding residues in any of SEQ ID NOS:2-6 as identifiable by global sequence alignment are also expressly provided. Preferred embodiments include or otherwise retain the WFFG (SEQ ID NO:46) motif, or at least aromatic residues such as tryptophane and tyrosine therein, the flanking repeating motifs, flexible linkers between the repeating motifs, or a combination thereof. In some embodiments, aromatic central region is mutated or removed. Thus, in some forms, one, two, three, four or more of the repeat domains are present and/or unmutated and/or some or all mutations are conservative; some or all of the positively charged residues are present and/or unmutated and/or some or all mutations are conservative; some or all of the aromatic residues are present and/or unmutated and/or some or all mutations are conservative are present and/or unmutated and/or some or all mutations are conservative; or a combination thereof in the disclosed adhesion domains.

As illustrated in FIG. 9E, exemplary variants that can adhere to glass include

(SEQ ID NO: 37)
YLGLEWATATVPYLGVEWATATVSYWFFGWATAQVAYLAPVWAEA
TIPYAVPVTLSK,
(SEQ ID NO: 38)
YLGLELKTKTVPLLGVELRTKTVSLWFFGLHTKOVALLAPVLKEK
TIPLAVPVTLSK,
and
(SEQ ID NO: 39)
YLGLEWKAKAVPYLGVEWRAKAVSYWFFGWHAKQVAYLAPVWKEK
AIPYAVPVTLSK,

c. Adhesive Domain Fusion Polypeptides

In some forms, the adhesive polypeptide is a fusion protein. It has been established that a 57 amino acids (57-aa) from the major biofilm adhesin Bap1 sequence can be incorporated into other recombinant proteins to direct and anchor proteins to surfaces, and adhesive domains engineered based on these findings are provided above. Therefore, fusion polypeptides including one or more adhesive domains and one or more heterologous amino acid sequences are provided.

As discussed in more detail below, the adhesive domain fusion polypeptides include any one or most of the adhesive domains provided herein. The adhesive domain fusion polypeptides, nucleic acids encoding the same, and delivery vehicles thereof typically include one or more heterologous amino acid sequences contiguous with the N and/or C terminus of the adhesive domain.

In some forms, the fusion polypeptides include an adhesive domain and one or more heterologous amino acid sequences (heterologous domain) that constitute a functional protein or domain (functional domain). In some forms, the fusion polypeptides include an entire endogenous protein fused to one or more adhesive domains. Exemplary schematics for the domain structure of a fusion protein include:
N-[Heterologous domain(s)]-[adhesive domain(s)]-C; or
N-[Adhesive domain(s)]-[Heterologous domain(s)]-C; or
N-[Heterologous domain(s)]-[CT domain(s)]-[Fusion peptide domain(s)]-C, where “N” and “C” refer to the ammino (NH2) and Carboxyl (COOH) termini, respectively.

The number of functional or heterologous domain(s) and adhesive domains can vary according to the requirements of the fusion peptide. For example, the domain structure of a fusion protein can include:
N-[Heterologous domain(s)]X-[Adhesive domain]Y-C; or
N-[Adhesive domain]X-[Heterologous domain(s)]Y-C; or
N-[Heterologous domain(s)]X-[Adhesive domain]Y-[Heterologous domain(s)]Z-C; or
N-[Adhesive domain]Y-[Heterologous domain(s)]Z-[Adhesive domain]W-C, Where W, X, Y and Z are independently between 0 and 10 provided at least one adhesive domain is present, and “N” and “C” refer to the amino (NH2) and Carboxyl (COOH) termini, respectively. In preferred forms, W, X, Y and Z are independently 0, 1, 2 or 3, provided at least one adhesive domain is present.

In some forms, an adhesive domain is linked to one or more heterologous domains by one or more linker domains, such as a flexible linker.

i. Heterologous Sequences

In some forms, the fusion polypeptides include a heterologous amino acid sequence that encodes a functional element. Functional elements that can be associated with, linked, conjugated, or otherwise attached directly or indirectly to the adhesive domain polypeptide sequence, are described. The heterologous polypeptide sequence is not derived from Vibrio cholerae Bap1, and in some forms is not derived from Vibrio cholerae.

In some forms, the heterologous amino acid sequence encodes a molecule such as a therapeutic or diagnostic molecule.

In some forms, the heterologous amino acid sequence encodes a molecule that improves a physicochemical property of the adhesive polypeptide. Exemplary properties that can be modulated or improved are selected from solubility, adhesion force, cross-linking, and improvement in protein expression, purification, recovery rate, and biodegradability of the adhesive protein.

In other forms, the heterologous amino acid sequence encodes a molecule that is designed to assist or enable the expression, folding, intracellular export or otherwise modify production of the adhesive domain. Such molecules include, but are not limited to, protein transduction domains, fusogenic peptides, targeting molecules, and sequences that enhance protein expression and/or isolation.

The polypeptides can optionally include additional sequences or moieties, including, but not limited to linkers and purification tags.

In a preferred form the purification tag is a polypeptide. Polypeptide purification tags are known in the art and include but are not limited to His tags which typically include six or more, typically consecutive, histidine residues; FLAG tags, which typically include the sequence DYKDDDDK (SEQ ID NO:8); haemagglutinin (HA) for example, YPYDVP (SEQ ID NO:9); MYC tag for example ILKKATAYIL (SEQ ID NO:10) or EQKLISEEDL (SEQ ID NO:11). Methods of using purification tags to facilitate protein purification are known in the art and include, for example, a chromatography step wherein the tag reversibly binds to a chromatography resin.

Purifications tags can be N-terminal or C-terminal to a protein. The purification tags N-terminal to the recombinant protein can be separated from the polypeptide of interest at the time of the cleavage in vivo. Therefore, purification tags N-terminal to the recombinant protein can be used to remove the recombinant protein from a cellular lysate following expression and extraction of the expression or solubility enhancing amino acid sequence, but cannot be used to remove the polypeptide of interest. Purification tags C-terminal to the recombinant protein can be used to remove the polypeptide of interest from a cellular lysate following expression of the recombinant protein, but cannot be used to remove the expression or solubility enhancing amino acid sequence. Purification tags that are C-terminal to the expression or solubility enhancing amino acid sequence can be N-terminal to, C-terminal to, or incorporated within the sequence of the polypeptide of interest.

In some forms, the heterologous sequence also has an adhesive function. For example, in some forms, the heterologous sequence adheres to a biotic surface. Exemplary proteins that can adhere to a biotic surface are known in the art and can be included in fusion proteins. In some forms, the heterologous domain includes a specific binding protein or fragment thereof, such as an antibody, or an antigen-binding fragment of an antibody. The antibodies can be those that specifically bind one or more molecules, cells or tissues in a desired location in vivo, or they can be polyclonal. Antibodies can include an antigen binding site that binds to an epitope on a target molecule, such as a protein. Typically, the antibodies bind to, but do not inhibit or alter the function of one or more target molecules by binding directly to the target molecule, its ligands or its accessory molecules. In some forms, the antibodies bind to and inhibit the function of one or more target molecules by binding directly to the target molecule, its ligands or its accessory molecules. Any specific antibody can be used in the adhesive fusion proteins. Various types of antibodies and antibody fragments can be used in the adhesive fusion proteins, including whole immunoglobulin of any class, fragments thereof, and synthetic proteins containing at least the antigen binding variable domain of an antibody. The antibody can be an IgG antibody, such as IgG1, IgG2, IgG3, or IgG4. An antibody can be in the form of an antigen binding fragment including a Fab fragment, F(ab′)2 fragment, a single chain variable region, and the like. Antibodies can be polyclonal or monoclonal (mAb). Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind a target antigen and/or exhibit the desired biological activity (U.S. Pat. No. 4,816,567; and Morrison, et al., Proc. Natl. Acad. Sci. USA, 81: 6851-6855 (1984)). Antibodies that are included in the adhesive fusion proteins can also be modified by recombinant means, for example by deletions, additions or substitutions of amino acids, to increase efficacy of the antibody in mediating the desired function. Substitutions can be conservative substitutions. For example, at least one amino acid in the constant region of the antibody can be replaced with a different residue (see, e.g., U.S. Pat. Nos. 5,624,821; 6,194,551; WO 9958572; and Angal, et al., Mol. Immunol. 30:105-08 (1993)). In some forms, changes are made to reduce undesired activities, e.g., complement-dependent cytotoxicity. The antibody can be a bi-specific antibody having binding specificities for at least two different antigenic epitopes. In one form, the epitopes are from the same antigen. In another form, the epitopes are from two different antigens. Bi-specific antibodies can include bi-specific antibody fragments (see, e.g., Hollinger, et al., Proc. Natl. Acad. Sci. U.S.A., 90:6444-48 (1993); Gruber, et al., J. Immunol., 152:5368 (1994)).

Antibodies can be generated by any means known in the art. Exemplary descriptions means for antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); and Current Protocols In Immunology (John Wiley & Sons, most recent edition). Fragments of intact Ig molecules can be generated using methods well known in the art, including enzymatic digestion and recombinant means.

In some forms, a heterologous domain included in the adhesive fusion proteins includes a specific binding protein or fragment thereof, such as biotin. For example, the heterologous domain polypeptide can be a biotinylated polypeptide. When the heterologous domain polypeptide is biotinylated, the conjugate can be formed through specific interaction with a streptavidin-conjugated active agent.

In some forms, a heterologous domain included in the adhesive fusion proteins includes a specific binding protein or fragment thereof, such as biotin. For example, the heterologous domain polypeptide can be a biotinylated polypeptide. When the heterologous domain polypeptide is biotinylated, the conjugate can be formed through specific interaction with a streptavidin-conjugated active agent.

In some forms, a heterologous domain included in the adhesive fusion proteins is or includes a lectin. Lectins are carbohydrate-binding proteins that mediate important cell-cell interactions including fertilization, pathogen invasion, and immune system activation or attenuation. Some cells express lectins to recognize and bind unique glycan displays on the surface of pathogens, facilitating highly efficient internalization of antigens at very low (nanomolar) concentrations. Additionally, lectins use glycan-mediated pathogen recognition to regulate cytokine secretion/gene expression for directed T cell activation. Therefore, in some forms, the heterologous domain included in the adhesive fusion proteins is or includes a lectin or is modified by attachment of ligands that bind lectins expressed at the surface of cells. In particular forms the heterologous domain included in the adhesive fusion proteins is or includes a lectin that binds to carbohydrates expressed at the surface of a target cell type or tissue in vivo. Therefore, in some forms, the adhesive fusion protein is targeted to, and binds to a specific target in vivo, as directed by lectin-mediated carbohydrate binding through the heterologous domain.

In some forms, the heterologous polypeptide is derived from a bacterium. In some forms, the heterologous polypeptide is derived from Vibrio Spp., such as Vibrio cholerae. In particular forms, the heterologous polypeptide is or is derived from a Biofilm matrix protein (RbmC). RbmC proteins adhere to biotic surfaces. Therefore, in some forms, the fusion includes one or more polypeptides derived from an RbmC protein, to provide a dual abiotic/biotic binding function, such as that exhibited by a “double-sided” adhesive tape.

An exemplary RbmC is the V. cholerae RbmC, having NCBI GenBank accession number WP000200580.1 and an amino acid sequence:

(SEQ ID NO: 7)
MTSHYIALAVGLLSLSSNVVQATTNEAEGCIISRLNGEKYCLKVG
ERSGYSLPSWIYAHPVDVQAPSGVSVMLSDWDNLSYNRLAVFDRY
TGNEDLKNVKAYNGAYLDFSKPRSMRVLASETYPEACIVSROTGE
RFCLKEGERSGYSLPAYIYGHEVDVEAPLGLGVMLSDWDNLSYNR
LAVFGGNTQNEQMRAVKAYNGETLDFSKPRSMRVVPYDGDSSALN
MKLKWSWQGSAFQPNSNQVMVTPIVAQLNDDNGDGKIDEKDVADL
IVVTFEGNKYANGGLVRALSGVDGSELWSYANGGVIADARYSPAV
GDLDGDGIVEIVITNNRDQFITILDNQGNIKKQIPTTESGWRIVG
DITLADLDHDGSVEILAADGVYNYHSGLVFNHPWAPSSINVDVDG
DQQQEVFSGGTLFQNNGAINWQYQANDAVWFSSLVNLDNDAEPEI
VASVPATFATGDNARFAVLEHDGTIKWEINNTANPGGGVQAVSNF
LGKAQAVETSEFSKVYGYQPNNNPASIALAVDGKISVRSGFAIDA
IGASASTLVGGTGGNLNAAVNVKDIKAIDLTWGKYYWGGYHLLAL
DFRMSNGSVISMGSKNYAYSKQTERFTVPAGSRIKGIKAWTAGWL
LDGVQFELATQNGTNDLDVKGIVYAGYAAVDMYNSKGERVWSVAN
DDTGSGKIGVSAYDFDNDGIDEVLVQDHARVRVLDGKTGKERASL
AHSTATLWEYPIVVDLEGDNNAELIVAANDFDRQYSINHGVYVYQ
SADSSKPWKNATRIWNQHAFHLTNINQDGTLPTFVEPSWLSHNTY
RSSTLRAAVGGESPIFGYSNTQQSQRVVTADNLMYLRSGFAIDAI
GTTVNNLVGGPVQGTNGGVLRAPIALDQLQSVEVTSGLYNWGGYH
IVAIKFTMKDGSSVLLGSTHYASNKKVETYTVPQGKRIKQINVWT
GGWLVEGFQFVY.

In some forms, the fusion peptide includes a fragment or variant of SEQ ID NO:7 that has the function of binding to a biotic surface. Exemplary fragments include 50, 75, 100, 125, 150, 175, 200, 225, 250, 275, 300, 325, 350, 375, 400, 425, 450, 475, 500, 525, 550, 575, 600, 625, 650, or 675 amino acids of SEQ ID NO:7, or a variant thereof such as a homolog thereof. Variants of fragments are also provided can be e.g., least 70% up to 99.9% identity e.g., 75%, 80%, 85%, 90%, or 95% identical to the wildtype fragment. Homologous RbmC proteins from other Vibrio species are also described. Exemplary polypeptides include the RbmC polypeptide from V. tubiashii, having NCBI GenBank accession number WP004748518.1; RbmC polypeptide from V. coralliilyticus, having NCBI GenBank accession number WP043010730.1; and RbmC polypeptide from V. mimicus, having NCBI GenBank accession number WP061052272.1, all of which are herein incorporated by reference in their entirety. See also De, et al., PLOS Pathog. 14, e1006841 (2018), Kaus, et al., J. Biol. Chem. 294, 14499-14511 (2019), which are specifically incorporated by reference herein in their entireties.

In some forms, the heterologous domain serves a primary function in enhancing the expression, folding and/or secretion of the adhesive polypeptide. In some forms, the heterologous polypeptide is stably expressed at high levels in solution by a mammalian, bacterial, insect or yeast expression system. In some forms, the heterologous polypeptide is readily expressed and secreted into the extracellular solution in a bacterial expression system. In some forms the heterologous polypeptide is readily expressed as inclusion bodies into the extracellular solution in a bacterial expression system. In some forms, the heterologous polypeptide is or includes an enzyme. Exemplary enzymes include endonuclease, exonuclease, extracellular protease, polysaccharide lyase, and extracellular chitinase protein. In some forms, the heterologous domain is or includes actin, tubulin, or a toxin.

### Linkers

In some forms, the recombinant protein includes one or more linkers or spacers. The term “linker” as used herein includes, without limitation, peptide linkers. The peptide linker can be any size provided it does not interfere with the binding of the epitope by the variable regions. In some forms, the linker includes one or more glycine and/or serine amino acid residues. In some forms, the linker includes a glycine-glutamic acid di-amino acid sequence. For example, a linker can include 4-8 amino acids. In a particular form, a linker includes the amino acid sequence GQSSRSS (SEQ ID NO:12). In another form, a linker includes 15-20 amino acids, for example 18 amino acids. Other flexible linkers include, but are not limited to, the amino acid sequences GSGSGS (SEQ ID NO:13), ASGGGS (SEQ ID NO:14), (Gly₄-Ser)₂ (SEQ ID NO:15) and (Gly₄-Ser)₄ (SEQ ID NO:16), and (GGGGS)₃ (SEQ ID NO:17).

The linkers can be used to link or connect two domains, regions, or sequences of a fusion protein.

Molecular biology techniques have developed so that therapeutic proteins can be genetically engineered to be expressed by microorganisms. The gram-negative bacterium, Escherichia coli, is a versatile and valuable organism for the expression of therapeutic proteins. Although many proteins with therapeutic or commercial uses can be produced by recombinant organisms, the yield and quality of the expressed protein are variable due to many factors. For example, heterologous protein expression by genetically engineered organisms can be affected by the size and source of the protein to be expressed, the presence of an affinity tag linked to the protein to be expressed, codon biasing, the strain of the microorganism, the culture conditions of microorganism, and the in vivo degradation of the expressed protein. Some of these problems can be mitigated by fusing the protein of interest to an expression or solubility enhancing amino acid sequence. Exemplary expression or solubility enhancing amino acid sequences include maltose-binding protein (MBP), glutathione S-transferase (GST), thioredoxin (TRX), NUS A, ubiquitin (Ub), and a small ubiquitin-related modifier (SUMO).

In some forms, the compositions disclosed herein include expression or solubility enhancing amino acid sequence. In some forms, the expression or solubility enhancing amino acid sequence is cleaved prior administration of the composition to a subject in need thereof. The expression or solubility enhancing amino acid sequence can be cleaved in the recombinant expression system, or after the expressed protein in purified.

Other examples of heterologous sequences that may improve expression of the adhesive domain and polypeptides formed therefrom include, but are not limited to:
adding a polyhistidine tag and/or a pelB secretion tag which is a signal that directs proteins to the periplasm;
adding a fragment of a well-behaved protein (e.g., FrhA);
constructing a Vibrio cholerae cytolysin (VCC), or another pore-forming toxin chimera. Pore-forming toxins are secreted in a water-soluble state, but then bind to membranes and form a transmembrane channel. An important feature of their function is that in the water-soluble state, they can shield a hydrophobic loop from the solvent and keep it soluble in solution. This hydrophobic loop is to be replaced with an adhesive domain whereby VCC may act as a molecular chaperone to produce soluble material.

E. coli strains can be constructed in which the adhesive domain is attached to an extracellularly secreted protein. In some forms, the fusion protein includes one or more proteins that act to anchor the adhesive domain to a cell, such as a target cell in vitro, or within an organism in vivo. Therefore, in some forms, the fusion protein includes one or more domains or polypeptides derived from one or more proteins that exist at the surface of cells, or within the membrane of cells. In some forms, the fusion protein includes one or more domains or polypeptides derived from one or more membrane proteins, or one or more cell-surface receptors, or one or more ligands thereof, effective to anchor the adhesive domain to the surface of a cell. In some forms, domains or polypeptides derived from one or more membrane proteins, or one or more cell-surface receptors, or one or more ligands thereof serve a dual function of targeting or locating the adhesive domain to a specific cell type of location in vivo.

Any of the fusion proteins can include one or more protease recognition sequence sites that can be targeted by a protease to cleave the fusion protein into individual domains (e.g., separate the adhesive domain from the heterologous sequence(s)). For example, in some forms, the fusion peptides include a trypsin protease recognition sequence, or a thrombin protease recognition sequence. An exemplary thrombin protease recognition sequence is LVPRGS (SEQ ID NO:18). Any specific protease recognition sequence known in the art can be included in the fusion proteins. In some forms, the protease recognition sequence is a TEV protease recognition sequence, such as ELNYFQ (S/G/A/M/C/H) (SEQ ID NO:19). Techniques and reagents to assist in expression of soluble proteins are known in the art, e.g., as described in Sheffield, et al., Protein Expr Purif 15, 34-39, the contents of which are hereby incorporated by reference in their entirety.

d. Exemplary Fusion Polypeptides and Conjugates

In some forms, the adhesive polypeptide and/or fusion polypeptide thereof is a fusion peptide designed for expression in V. cholerae for extracellular secretion, having one of the following configurations.

In some forms, the adhesive polypeptide and/or fusion polypeptide thereof is expressed as a C-terminal adduct in a fusion peptide including a V. cholerae extracellular protease (hapA) with amino acid sequence

(SEQ ID NO: 20)
MKMIQRPLNWLVLAGAATGFPLYAAQMVTIDDASMVEQALAQQQY
SMMPAASGFKAVNTVQLPNGKVKVRYQQMYNGVPVYGTVVVATES
SKGISQVYGQMAQQLEADLPTVTPDIESQQAIALAVSHFGEQHAG
ESLPVENESVQLMVRLDDNQQAQLVYLVDFFVASETPSRPFYFIS
AETGEVLDQWDGINHAQATGTGPGGNQKTGRYEYGSNGLPGFTID
KTGTTCTMNNSAVKTVNLNGGTSGSTAFSYACNNSTNYNSVKTVN
GAYSPLNDAHFFGKVVEDMYQQWLNTSPLTFQLTMRVHYGNNYEN
AFWDGRAMTFGDGYTRFYPLVDINVSAHEVSHGFTEQNSGLVYRD
MSGGINEAFSDIAGEAAEYFMRGNVDWIVGADIFKSSGGLRYFDQ
PSRDGRSIDHASQYYSGIDVHHSSGVFNRAFYLLANKSGWNVRKG
FEVFAVANQLYWAPNSTFDQGGCGVVKAAQDLNYNTADVVAAFNT
VGVNASCGTTPPPVGKVLEKGKPITGLSGSRGGEDFYTFTVTNSG
SVVVSISGGTGDADLYVKAGSKPTTSSWDCRPYRSGNAEQCSISA
VVGTTYHVMLRGYSNYSGVTLRLDYLGLEWKTKTVPYLGVEWRTK
TVSYWFFGWHTKQVAYLAPVWKEKTIPYAVPVTLSK.
The 57 amino acid polypeptide of
SEQ ID NO: 1 is underlined.

In some forms, the adhesive polypeptide and/or fusion polypeptide thereof is expressed as a C-terminal adduct in a fusion peptide including a V. cholerae extracellular protease (ivaP) with amino acid sequence

(SEQ ID NO: 21)
MFKKFLSLCIVSTFSVAATSALAQPNQLVGKSSPQQLAPLMKAAS
GKGIKNQYIVVLKQPTTIMSNDLQAFQQFTQRSVNALANKHALEI
KNVFDSALSGFSAELTAEQLQALRADPNVDYIEQNQIITVNPIIS
ASANAAQDNVTWGIDRIDQRDLPLNRSYNYNYDGSGVTAYVIDTG
IAFNHPEFGGRAKSGYDFIDNDNDASDCQGHGTHVAGTIGGAQYG
VAKNVNLVGVRVLGCDGSGSTEAIARGIDWVAQNASGPSVANLSL
GGGISQAMDQAVARLVQRGVTAVIAAGNDNKDACQVSPAREPSGI
TVGSTTNNDGRSNFSNWGNCVQIFAPGSDVTSASHKGGTTTMSGT
SMASPHVAGVAALYLQENKNLSPNQIKTLLSDRSTKGKVSDTQGT
PNKLLYSLTDNNTTPNPEPNPQPEPQPQPDSQLTNGKVVTGISGK
QGELKKFYIDVPAGRRLSIETNGGTGNLDLYVRLGIEPEPFAWDC
ASYRNGNNEVCTFPNTREGRHFITLYGTTEFNNVSLVARYYLGLE
WKTKTVPYLGVEWRTKTVSYWFFGWHTKQVAYLAPVWKEKTIPYA
VPVTLSK.
The 57 amino acid polypeptide of
SEQ ID NO: 1 is underlined.

In some forms, the adhesive polypeptide and/or fusion polypeptide thereof is expressed as a C-terminal adduct in a fusion peptide including a V. cholerae biofilm matrix protein (RbmA) with amino acid sequence

(SEQ ID NO: 22)
MSNFKGSIMNKRHYYLASCLALLFSTASYAEVDCELQPVIEANLS
LNQNQLASNGGYISSQLGIRNESCETVKFKYWLSIKGPEGIYFPA
KAVVGVDTAQQESDALTDGRMLNVTRGFWVPEYMADGKYTVSLQV
VAENGKVFKANQEFVKGVDLNSLPELNGLTIDIKNQFGINSVEST
GGFVPFTVDLNNGREGEANVEFWMTAVGPDGLIIPVNAREKWVIA
SGDTYSKVRGINFDKSYPAGEYTINAQVVDIVSGERVEQSMTVVK
KYLGLEWKTKTVPYLGVEWRTKTVSYWFFGWHTKQVAYLAPVWKE
KTIPYAVPVTLSK.
The 57 amino acid polypeptide of SEQ ID NO: 1
is underlined.

In some forms, the adhesive polypeptide and/or fusion polypeptide thereof is expressed as a C-terminal adduct in a fusion peptide including a V. cholerae extracellular polysaccharide lyase protein (RbmB) with amino acid sequence

(SEQ ID NO: 23)
MLLYLNQFNKEGGILRYIFISLVLFSPAIFSEVNSHNVIEYGAIA
NDGEDDSNAFQHALNQLNNGDALIIPTGEYQICKTLYLKEKNNIE
IIGSINSKLKKCRSENGEYLLHITYTQNLKIQGLSFEGLNNGDLK
PLWGEQGVYLGSTKGTLVVONQFARFGDAALRMTTASQDHSIPPG
SMAIKVSHNHFEDCAQVTTTQATAGTEMHGTQDIIIDNNQFNACK
LKLSARADTRGAKVINNQFENINGTSNEVSYYSDVYYSGNTFLNI
NGFAINIYPNSRTEQNVQWGNISIIGNTFDAIQQGIRLQSFSIND
PNNQSIKNIQISDNTFENIYFGNEIESQYKAIIRTNSQDNLVSFE
HVNITGNQYQLTPYSKFISIDHKSKLINIONNERIYSDHYYIQHF
IKDYLGLEWKTKTVPYLGVEWRTKTVSYWFFGWHTKQVAYLAPVW
KEKTIPYAVPVTLSK.
The 57 amino acid polypeptide of SEQ ID
NO:1 is underlined.

In some forms, the adhesive polypeptide and/or fusion polypeptide thereof is expressed as a C-terminal adduct in a fusion peptide including a V. cholerae extracellular endonuclease protein (Dns) with amino acid sequence

(SEQ ID NO: 24)
MMIFRFVTTLAASLPLLTFAAPISFSHAKNEAVKIYRDHPVSFYC
GCEIRWQGKKGIPDLESCGYQVRKNENRASRIEWEHVVPAWQFGH
QLQCWQQGGRKNCTRISPEFNQMEADLHNLTPAIGEVNGDRSNFS
FSQWNGVDGVTYGQCEMQVNFKERTAMPPERARGAIARTYLYMSE
QYGLRLSKAQSQLMQAWNNQYPVSEWECVRDQRIEKVOGNSNRFV
REQCPNYLGLEWKTKTVPYLGVEWRTKTVSYWFFGWHTKQVAYLA
PVWKEKTIPYAVPVTLSK.
The 57 amino acid polypeptide of SEQ ID NO:1 is
underlined.

In some forms, the adhesive polypeptide and/or fusion polypeptide thereof is expressed as a C-terminal adduct in a fusion peptide including a V. cholerae extracellular exonuclease protein (Xds) with amino acid sequence

(SEQ ID NO: 25)
MEMRTTPNLTRSTLALAISFGLVAPSYADLLISQYVEGSSENKAI
EIANTSDQTVSLNGYQLAMSTNGSGTWDKTLPLDGQVIAARDVLV
IAHGSANSAILATADLINNTVVNFNGNDPIALLNSDGSVHDVVGS
MGGADFAKDNTLARTTLTPSATYQASDWATQGKDNIDGLGALDTT
TPPSAFNCTLDGAEPSFTTIQQIQGEGSTSPYIQGYPYITNEDFF
VKGVVSAVTTGLTKGFYLQSLEDDYNPNTSEGLFVFTNQSSSDLA
PGDVVCVKGKVQEYYNLTQLKAENNQWVKQGQQAAPQAQAIEILP
SDEHFAQTLERYEGMLVKTTPELDMRVTRTFGYDYASRRNNMVLA
QGRINMQPNQQHPAGSEQASQQKLDNAQRRLFVESDAKAPDGQIP
YYPTFGRTDVDQDGSTEDYIRIDDTVSGLEGVVSYSYNEYRLIVT
NTISAENLVHNAPRQAKPDLDEGDLRIATFNVLNYFNSPFGGDAN
QHGDNRGANNLAEFEVQQAKIVNAIVRLDADIVGLMEIENNGFGE
GSAIAQLVNQINSQIADKKKHYRFVAIDSNGDGKTDAADSLGTDV
ITTGVIYRDKVVKLAQNRVIPMPSQQAPEVVDANGKVIEDGKNYQ
RDTLAPTFKVKGGNEKITVAVNHFKSKGSACWEDAAPVEQGGQAG
KDLDYQGACENFRVAAAVALGDALAKIDGHKVILGDMNSYGMEDP
MLVLTDYTPEKYGKTIRAARNTYIAGLEQFGDAGAEIKHSYGYLN
AVAMKHPDSWSYSENDEVGALDHLLVSPSLKHKVVDATDWHINGA
ESTLFDYNDEFKGNLPKYKDQFRASDHDPAVLELNIYGGSLGLGA
LLGLLGLGVWRRRRYLGLEWKTKTVPYLGVEWRTKTVSYWFFGWH
TKQVAYLAPVWKEKTIPYAVPVTLSK.
The 57 amino acid polypeptide of
SEQ ID NO: 1 is underlined.

In some forms, the adhesive polypeptide and/or fusion polypeptide thereof is expressed as a C-terminal adduct in a fusion peptide including a V. cholerae extracellular chitinase protein (chiA-1) with amino acid sequence

(SEQ ID NO: 26)
MKRYCLAAVITASLGVSYSAQAYNCAGVPVWDSSTVYVGSDKVQKINTAYQARYWTQ
GNDPVTHSGQWDAWQILGQCDGGANNPPQVSIQSPLNNAKIPQGSVVGLQANASDSD
GSITQVEFLVGTQRIAIDQQAPYQVDWTATLGATSVTAIATDNQGATTSSTVNISVT
PTGNPVPPTVTLTSPTGSEQLTVGDVLAVAANATDSDGTVNAVEFYVDGQLVVIDSS
EPYQFNWNAAVGSHTFKAKAIDNDNLSTLSQEVTLTVGSGSNAGCAGLPVYSVGTAY
SAGQLVQNKNQKYRCDIAGWCSSSSGWAYEPGVGSYWKEAWSGLGACSTPPVVTLIN
PTANQVILAGSTVSVAAQASDADGSVMQVEFFAGNNSLGVVTQAPYAVNWLATTTGN
QTLKAVATDNDSNTSESAVSVTVSDQDLVVSLTSPTSGQTVGLGKPVNIAADATSLT
NNVAKVEFVVNGAVVATDTTEPFAYSWTPSAIGNYTVAAKATDAAGTSVTSSAAAIS
VVEQAQKKHRLIGYWHNFVNGAGCPIRLADMSQAWDVIDIAFAENDRNSTGTVHFNL
YAGDIYSSCPALDPAQFKQDMKALQAKGKVFVLSLGGAEGTITLNTDQDEANFVSSL
TALIKEWGFDGLDVDLESGSNLVHGSQIQARLGRALKQIEKNIGGDMFLTMAPEHPY
VQGGMVAYSGIWGAYIPVINEVRDTLDILHVQLYNNGGLPNPYTPSAAPEGSVDMMV
AQSKMLIEGFTLANGTRFEPLRDDQVAIGLPSGPSSANSGQAPTQNILDALDCLTKG
TRCGTIKPAFAYPNYAGVMTWSINWDKHDGFNFSKPVGDKLSQMNNAQYLGLEWKTK
TVPYLGVEWRTKTVSYWFFGWHTKQVAYLAPVWKEKTIPYAVPVTLSK.
The 57 amino acid polypeptide of SEQ ID NO: 1 is underlined.

In some forms, the adhesive polypeptide and/or fusion polypeptide thereof is expressed as an integral component of a V. cholerae biofilm matrix protein (RbmC) with amino acid sequence

(SEQ ID NO: 27)
MTSHYIALAVGLLSLSSNVVQATTNEAEGCIISRLNGEKYCLKVGERSGYSLPSWIY
AHPVDVQAPSGVSVMLSDWDNLSYNRLAVFDRYTGNEDLKNVKAYNGAYLDFSKPRS
MRVLASETYPEACIVSRQTGERFCLKEGERSGYSLPAYIYGHEVDVEAPLGLGVMLS
DWDNLSYNRLAVFGGNTQNEQMRAVKAYNGETLDFSKPRSMRVVPYDGDSSALNMKL
KWSWQGSAFQPNSNQVMVTPIVAQLNDDNGDGKIDEKDVADLIVVTFEGNKYANGGL
VRALSGVDGSELWSYANGGVIADARYSPAVGDLDGDGIVEIVTINNRDQFITILDNQ
GNIKKQIPTTESGWRIVGDITLADLDHDGSVEILAADGVYNYHSGLVENHPWAPSSI
NVDVDGDQQQEVFSGGTLFQNNGAINWQYQANDAVWFSSLVNLDNDAEPEIVASVPA
TFATGDNARFAVLEHDGTIKWEINNTANPGGGVQAVSNFLGKAQAVETSEFSKVYGY
QPNNNPASIALAVDGKISVRSGFAIDAIGASASTLVGGTGGNLNAAVNVKDIKAIDL
TWGKYYWGGYHLLALDFRMSNGSVISMGSKNYAYSYLGLEWKTKTVPYLGVEWRTKT
VSYWFFGWHTKQVAYLAPVWKEKTIPYAVPVTLSKKQTERFTVPAGSRIKGIKAWTA
GWLLDGVQFELATQNGTNDLDVKGIVYAGYAAVDMYNSKGERVWSVANDDTGSGKIG
VSAYDFDNDGIDEVLVQDHARVRVLDGKTGKERASLAHSTATLWEYPIVVDLEGDNN
AELIVAANDFDRQYSINHGVYVYQSADSSKPWKNATRIWNQHAFHLTNINQDGTLPT
FVEPSWLSHNTYRSSTLRAAVGGESPIFGYSNTQQSQRVVTADNLMYLRSGFAIDAI
GTTVNNLVGGPVQGTNGGVLRAPIALDQLQSVEVTSGLYNWGGYHIVAIKFTMKDGS
SVLLGSTHYASNKKVETYTVPQGKRIKQINVWTGGWLVEGFQFVY.
The 57 amino acid polypeptide of SEQ ID NO: 1 is underlined.

In some forms, the adhesive polypeptide and/or fusion polypeptide thereof is expressed as an integral component of a V. cholerae extracellular toxin protein (VCC/HlyA) with amino acid sequence

(SEQ ID NO: 28)
MPKLNRCAIAIFTILSAISSPTLLANINEPSGEAADIISQVADSHAIKYYNAADWQA
EDNALPSLAELRDLVINQQKRVLVDFSQISDAEGQAEMQAQFRKAYGVGFANQFIVI
TEHKGELLFTPFDQAEEVDPQLLEAPRTARLLARSGFASPAPANSETNTLPHVAFYI
SVNRAISDEECTFNNSWLWKNEKGSRPFCKDANISLIYRVNLERSLQYGIVGSATPD
AKIVRISLDDDSTGAGIHLNDQLGYRQFGASYTTLDAYFREWSTDAIAQDYRFVFNA
SNNKAQILKTFPVDNINEKFERKEVSGFELGVTGGVEVSGDGPKAKLEARASYTQSR
WLTYNTQDYRIERNAKNAQAVSFTWNRQQYATAESLLNRSTDALWVNTYPVDVNRIS
PLSYASFVPKMDVIYKASATETGSTDFIIDSSVNIRPIYNGAYKHYYVVGAHQSYHG
FEDTPRRRITKSASFTVDWDHPVFTGGRPVNLQLASFNNRCIQVDAQGRLAANTCDS
QQSAQSFIYDQLGRYVSASNTKLCLDGEALDALQPCNQNLTQRWEWRKGTDELINVY
SGESLGHDKQTGELGLYASSNDAVSLRTITAYTDVFNAQESSPILGYTQGKMNQQRV
GQDHRLYVRAGAAIDALGSASDLLVGGNGGSLSSVDLSGVKSITATSGDFQYGGQQL
VALTFTYQDGRQQTVGSKAYVINYLGLEWKTKTVPYLGVEWRTKTVSYWFFGWHTKQ
VAYLAPVWKEKTIPYAVPVTLSKAHEDREDLPAAAKITQLKIWSDDWLVKGVQFDLN.
The 57 amino acid polypeptide of SEQ ID NO: 1 is underlined.

In some forms, the adhesive polypeptide and/or fusion polypeptide thereof is expressed as a C-terminal adduct in a fusion peptide including Actin protein with amino acid sequence

(SEQ ID NO: 29)
MDDDIAALVVDNGSGMCKAGFAGDDAPRAVFPSIVGRPRHQGVMVGMGQKDSYVGDE
AQSKRGILTLKYPIEHGIVTNWDDMEKIWHHTFYNELRVAPEEHPVLLTEAPLNPKA
NREKMTQIMFETFNTPAMYVAIQAVLSLYASGRITGIVMDSGDGVTHTVPIYEGYAL
PHAILRLDLAGRDLTDYLMKILTERGYSFTTTAEREIVRDIKEKLCYVALDFEQEMA
TAASSSSLEKSYELPDGQVITIGNERFRCPEALFQPSFLGMESCGIHETTENSIMKC
DVDIRKDLYANTVLSGGTTMYPGIADRMQKEITALAPSTMKIKIIAPPERKYSVWIG
GSILASLSTFQQMWISKQEYDESGPSIVHRKCFYLGLEWKTKTVPYLGVEWRTKTVS
YWFFGWHTKQVAYLAPVWKEKTIPYAVPVTLSK. The 57 amino acid polypeptide
of SEQ ID NO: 1 is underlined.

In some forms, the adhesive polypeptide and/or fusion polypeptide thereof is expressed as a C-terminal adduct in a fusion peptide including Tubulin protein with amino acid sequence

(SEQ ID NO: 30)
MRECISIHVGQAGVQIGNACWELYCLEHGIQPDGQMPSDKTIGGGDDSFNTFFSETG
AGKHVPRAVFVDLEPTVIDEVRTGTYRQLFHPEQLITGKEDAANNYARGHYTIGKEI
IDLVLDRIRKLADQCTGLQGFSVFHSFGGGTGSGFTSLLMERLSVDYGKKSKLEFSI
YPAPQVSTAVVEPYNSILTTHTTLEHSDCAFMVDNEAIYDICRRNLDIERPTYTNLN
RLIGQIVSSITASLRFDGALNVDLTEFQTNLVPYPRAHFPLATYAPVISAEKAYHEQ
LSVAEITNACFEPANQMVKCDPRHGKYMACCLLYRGDVVPKDVNAAIATIKTKRTIQ
FVDWCPTGFKVGINYEPPTVVPGGDLAKVQRAVCMLSNTTAIAEAWARLDHKFDLMY
AKRAFVHWYVGEGMEEGEFSEAREDMAALEKDYEEVGVDSVEGEGEEEGEEYYLGLE
WKTKTVPYLGVEWRTKTVSYWFFGWHTKQVAYLAPVWKEKTIPYAVPVTLSK.
The 57 amino acid polypeptide of SEQ ID NO: 1 is underlined.

In other forms, the adhesive polypeptide and/or fusion polypeptide thereof is expressed as part of a fusion protein in a bacterial expression system. Suitable bacterial expression systems include E. coli. Therefore, in some forms, the adhesive polypeptide and/or fusion polypeptide thereof is expressed as a C-terminal adduct to a C-terminal fragment of FrhA a fusion peptide. In some forms, the fusion protein includes one or more additional peptide motifs to assist the isolation and/or purification of the expressed protein. An exemplary motif is a poly-histidine tag. In some forms, the fusion peptides include a cleavage site, for example, a protease recognition sequence, e.g., for removal of the one or more additional peptide motifs to assist the isolation and/or purification of the expressed protein and thrombin cleavage site from pET28b.

In some forms, the adhesive polypeptide and/or fusion polypeptide thereof is expressed as a C-terminal adduct to a C-terminal fragment of FrhA a fusion peptide having a N-terminal histidine tag and protease cleavage sequence with amino acid sequence

(SEQ ID NO: 31)
MGSSHHHHHHSSGLVPRGSHMASMTGGQQMGRDPSGLNGANAELTIHESNLSGGSSP
DHSALSKVGSFSFTSIDGLASLVIAGHSFNVAELLALNSSEATISTPYGELTLTGFS
GDLTGGTVQYEYTLNENVDNDSATNANDTDYTDHISVTVIDVDGSQITDSLDVKIVD
DASTALDDRGEVDIVADSFTVSGVVANWTSWSNGTNVTTFDGINAPNGGGLDNDSGK
DQIRWGQPASSYSSGYGFIDNDSALNGEFALNQDIILGTFTHYNYPVYSGGAITSAS
MDVAFSVTDAHGVLTPVTLKLNFDHNETPNTNNPEASKDIIKVGNTNVTFENAGALY
TLQVIGFRIPGTNQIVTEIRTGENATNSYELVVRVGPGEGYELPSTSGNVLSNDVSG
ADVDMTVVGAASGNHVSSGVSGSVGSMIAGLYGNLILLADGSYTYQVTANASSIPND
AIEIFTYTMKDGDGDTSTALLSINVNRVTMADFNANQDHKVGLEDTVVAGNVLDNDG
SKNTSVDHFTVGNDATSHVVGSPVSLEQGELTLNSDGSYTFKPADNWNGEVPVITYT
THTGKTSTLAITITPVDDATVTQPDHKSIAEDTIATGNVLANDCDIDSALSVTSFHV
EGVNGVYTAGNTMYQLAEGTLVLKANGDYTFDPKDNWSGSLPEITYTTNTGATGTLN
IHVEAVADVPNLTINGYTSVAAINFEDARLNGSWDGVVANQIKGLNTIGTWHISNNS
GKVEIGYENIYVSGGSSTNKVMEIEFNNGDKTLYTDIHAQAGRFYELDFDIAARAGS
VNSSGLTIKLVPLNAYGVPILAEAITLYDENPTNANWLRDQKVTLPIDQTGEYRLLF
ESDDANSYGAILDNLAFKVVDNMGYRGDFIKLSEISTSLNDTDTSETLSLKLKGMPE
GSILKDDKGHEVTVGSNGEVDITGWDYSSLQIKTPNHGNFNITVEATATESSNQDSA
TTSATIPVTVLHPNEYLGRGGVDSFLLTKSNGDNANLNIALNAYYEGTTAVAPVTQQ
VAVTIDTDLVIHSGNSNDYIDLGISRADNTVYTGSSIPNENNSTPSQSTLADSAFMK
NDVITDHDGVLLQSVQSQIQPITDTVNLGSGNDTVYGGGGNLAAYGGAGNDTLIGGD
GNDALRGGADNDYLSGGRGNDVLRGDSGNDVLIGGLGHDILTGGSGEDLFKWVDGDL
DGSTDRITDFHLSEKDKIDLSDLEDNPSEQEVTALLDSIKSTVQGDDHSSSFKVEKN
DGSSVTIQLDGVSSVELINNLASIIQIKEDLEYLGLEWKTKTVPYLGVEWRTKTVSY
WFFGWHTKQVAYLAPVWKEKTIPYAVPVTLSK.
The 57 amino acid polypeptide of SEQ ID NO: 1 is underlined. The
histidine tag motif and protease cleavage sequence are indicated
in italics.

All of the expressly provided fusion protein sequences also expressly provided wherein the underlined sequence is replaced with alternative adhesion domain as described herein and/or wherein the adhesion domain is additionally or alternatively at the N-terminus of the protein.

In some forms, the adhesive polypeptide and/or fusion polypeptide thereof is expressed as an integral component of a VCC fusion peptide having a N-terminal histidine tag and protease cleavage sequence with amino acid sequence

(SEQ ID NO: 32)
MSYYHHHHHHDYDIPTTENLYFQGAMGSNINEPSGEAADIISQVADSHAIKYYNAAD
WQAEDNALPSLAELRDLVINQQKRVLVDFSQISDAEGQAEMQAQFRKAYGVGFANQF
IVITEHKGELLFTPFDRTEEIDPALLEAPRTAALLGRSGFASPAPANSETNTLPHVA
FYISVNRAISDEECTFNNSWLWKNEKGSRPFCKDANISLIYRVNLERSLQYGIVGSA
TPDAKIVRISLDDDSTGAGIHLNDQLGYRQFGASYTTLDAYFREWSTDAIAQDYRFV
FNASNNKAQILKTFPVDNINASYLGLEWKTKTVPYLGVEWRTKTVSYWFFGWHTKQV
AYLAPVWKEKTIPYAVPVTLSKGTTYNTQDYRIERNAKNAQAVSFTWNRQQYATAES
LLNRSTDALWVNTYPVDVNRISPLSYASFVPKMDVIYKASATETGSTDFIIDSSVNI
RPIYNGAYKHYYVVGAHQSYHGFEDTPRRRITKSASFTVDWDHPVFTGGRPVNLQLA
SFNNRCIQVDAQGRLTANMCDSQQSAQSFIYDQLGRYVSASNTKLCLDGAALDALQP
CNQNLTQRWEWRKGTDELTNVYSGESLGHDKQTGELGLYASSNDAVSLRTITAYTDV
FNAQESSPILGYTQGKMNQQRVGQDNRLYVRAGAAIDALGSASDLLVGGNGGSLSSV
DLSGVKSITATSGDFQYGGQQLVALTFTYQDGRQQTVGSKAYVTNAHEDREDLPDAA
KITQLKIWADDWLVKGVQFDLN. The 57 amino acid polypeptide of SEQ ID
NO: 1 is underlined. The histidine tag motif and protease cleavage
sequence are indicated in italics.

In other forms, the adhesive polypeptide and/or fusion polypeptide thereof is expressed as a stand-alone polypeptide with one or more adjunct amino acids to assist expression, e.g., with amino acid sequence

(SEQ ID NO: 33)
MDYLGLEWKTKTVPYLGVEWRTKTVSYWFFGWHTKQVAYLAPVWKEKT
IPYAVPVTLSK.

In other forms, the adhesive polypeptide and/or fusion polypeptide thereof is expressed as a polypeptide with one or more adjunct amino acid sequences, such as a PelB sequence secretion signal and C-terminal polyhistidine tag, e.g., with amino acid sequence

(SEQ ID NO: 34)
MKYLLPTAAAGLLLLAAQPAMAMDYLGLEWKTKTVPYLGVEWRTKTVS
YWFFGWHTKQVAYLAPVWKEKTIPYAVPVTLSKLEHHHHHH.

In other forms, the adhesive polypeptide and/or fusion polypeptide thereof is expressed as a polypeptide with one or more adjunct amino acid sequences, such as a thrombin protease recognition sequence and N-terminal polyhistidine tag, e.g., with amino acid sequence

(SEQ ID NO: 35)
MGSSHHHHHHSSGLVPRGSHMYLGLEWKTKTVPYLGVEWRTKTVSYWF
FGWHTKQVAYLAPVWKEKTIPYAVPVTLSK.

e. Complexation and Modification

In some forms, the adhesive polypeptide and/or fusion polypeptide thereof is bound to, conjugated with, or otherwise associated with one or more additional heterologous molecules or moieties.

Exemplary heterologous molecules or moieties are selected from an amino acid, a protein, a nucleic acid, a carbohydrate, a lipid, a metal, a polymer, a cell, a virion, a small molecule, and a mineral, or combinations thereof. In some forms, the additional molecules is a drug, a diagnostic agent, a prophylactic agent, or combinations thereof. In some forms, the additional molecule is a therapeutic agent. In some forms, the additional molecule is an imaging agent or a dye. In some forms, the additional agent is a tag, such as a barcode, or a fluorescent, magnetic or radioactive tag. Additional agents can be conjugated to the adhesive peptides using any system known in the art for conjugation of polypeptides with additional agents.

In other forms, the adhesive polypeptide and/or fusion polypeptide thereof is modified by chemical modification of one or more or the amino acids within the polypeptide. The modification can be biological posttranslational modification, for example, within a cell or solution from which the polypeptide is expressed or produced, or it may be synthetically modified, for example, by chemical modification. One or more amino acids are modified. In some forms, all of the amino acids in the polypeptide are modified. In other forms, only one or more types of amino acid are modified, whilst other amino acids are non-modified. The amount of modified residues may be expressed as a % value of the total (100%) of the residues in the sequence. In some forms, 10%, 20%, 30%, 40%, 50%, 60%, 70%, 80%, 90%, or more than 90%, such as 95% or 99% of the total number of amino acid residues within the sequence are modified. In some forms, amino acids are modified according to one or more characteristic, for example, in some forms, basic or neutral or acidic, hydrophobic or hydrophilic residues are modified. Exemplary types of modification include lipidation, phosphorylation, hydroxylation, crosslinking, acetylation, ubiquitination, methylation, glycosylation, succinylation, methylation, malonylation, sumoylation, nitrosylation, amidation, oxydation, and sulfation. Therefore, in some forms, an adhesive domain, or a polypeptide including an adhesive domain is modified such that one or more residues within the domain or polypeptide including the domain is lipidated, phosphorylated, hydroxylated, crosslinked, acetylated, ubiquitinated, methyled, glycosylated, succinylation, methylated, malonylated, sumoylated, nitrosylated, amidated, oxydated, or sulfated, or combinations thereof.

### 2. Nucleic Acids

Isolated Nucleic acids expressing or encoding the adhesive domain polypeptides derived from Vibrio cholerae are also described.

a. Isolated Nucleic Acid Molecules

Isolated nucleic acid sequences encoding the disclosed adhesive polypeptides are provided.

As used herein, “isolated nucleic acid” refers to a nucleic acid that is separated from other nucleic acid molecules that are present in a genome, including nucleic acids that normally flank one or both sides of the nucleic acid in a bacterial genome. An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation or can be complementary to a reference sequence encoding an adhesive domain polypeptide. Thus, nucleic acids encoding SEQ ID NOS:1-6, and fragments and variants thereof, in sense and antisense, and in single stranded and double stranded forms, are provided.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

b. Vectors

The application also relates to vectors including an isolated polynucleotide encoding an adhesive polypeptide and/or fusion polypeptides. As used herein, a “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). A vector can be a DNA vector or an RNA vector. In some forms, a vector is a DNA plasmid. One of ordinary skill in the art can construct a vector of the application through standard recombinant techniques in view of the present disclosure.

A vector of the application can be an expression vector. As used herein, the term “expression vector” refers to any type of genetic construct including a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

Vectors can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e., mRNA) into the host cell or organism. In the context of the disclosure, this term encompasses promoters, enhancers and other expression control elements (e.g., polyadenylation signals and elements that affect mRNA stability).

In some forms, the vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g., a linear covalently closed DNA, e.g., a linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically include an origin of replication, a multiple cloning site, and a selectable marker, which for example, can be an antibiotic resistance gene. Examples of suitable DNA plasmids that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC®. Complete baculovirus expression system (Thermo Fisher Scientific), which can be used for production and/or expression in insect cells; pcDNA™. Or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in bacterial, yeast, insect or mammalian host cells. Expression vectors suitable for protein expression in mammalian host cells include, but are not limited to, pcDNA™, pcDNA3™, pVAX, pVAX-1, ADVAX, NTC8454, etc. In some forms, an expression vector is based on pVAX-1, which can be further modified to optimize protein expression in mammalian cells. pVAX-1 is a commonly used plasmid in DNA vaccines, and contains a strong human immediate early cytomegalovirus (CMV-IE) promoter followed by the bovine growth hormone (bGH)-derived polyadenylation sequence (pA). pVAX-1 further contains a pUC origin of replication and a kanamycin resistance gene driven by a small prokaryotic promoter that allows for bacterial plasmid propagation.

The vector can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, arenavirus viral vectors, replication-deficient arenavirus viral vectors or replication-competent arenavirus viral vectors, bi-segmented or tri-segmented arenavirus, infectious arenavirus viral vectors, nucleic acids which include an arenavirus genomic segment wherein one open reading frame of the genomic segment is deleted or functionally inactivated (and replaced by a nucleic acid encoding an adhesive polypeptide), arenavirus such as lymphocytic choriomeningitidis virus (LCMV), e.g., clone 13 strain or MP strain, and arenavirus such as Junin virus e.g., Candid #1 strain, etc.

In some forms, the viral vector is an adenovirus vector, e.g., a recombinant adenovirus vector. A recombinant adenovirus vector can for instance be derived from a human adenovirus (HadV, or AdHu), or a simian adenovirus such as chimpanzee or gorilla adenovirus (ChAd, AdCh, or SadV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other forms, an adenovirus vector is a rhAd vector, e.g. rhAd51, rhAd52 or rhAd53. A recombinant viral vector can be prepared using methods known in the art in view of the present disclosure. For example, in view of the degeneracy of the genetic code, several nucleic acid sequences can be designed that encode the same polypeptide. A polynucleotide encoding a adhesive polypeptide can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

The vectors, e.g., a DNA plasmid or a viral vector (particularly an adenoviral vector), can include any regulatory elements to establish conventional function(s) of the vector, including but not limited to replication and expression of the adhesive polypeptide encoded by the polynucleotide sequence of the vector.

c. Regulatory Elements

Any of the disclosed nucleic acids, including RNAs and DNAs such as DNA vectors can include one or more regulatory elements.

Regulatory elements include, but are not limited to, a promoter, an enhancer, a polyadenylation signal, translation stop codon, a ribosome binding element, a transcription terminator, selection markers, origin of replication, etc. An isolated include acid can be, and a vector can include, one or more expression cassettes. An “expression cassette” is part of a nucleic acid such as a vector that directs the cellular machinery to make RNA and protein. An expression cassette typically includes three components: a promoter sequence, an open reading frame, and a 3-untranslated region (UTR) optionally including a polyadenylation signal. An open reading frame (ORF) is a reading frame that contains a coding sequence of a protein of interest (e.g., an adhesive polypeptide) from a start codon to a stop codon. Regulatory elements of the expression cassette can be operably linked to a polynucleotide sequence encoding an adhesive polypeptide.

As used herein, the term “operably linked” is to be taken in its broadest reasonable context and refers to a linkage of polynucleotide (or polypeptide, etc.) elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

i. Promotors

The disclosed nucleic acids, including vectors, can include a promoter sequence, preferably within an expression cassette, to control expression of a adhesive polypeptide or adhesive fusion polypeptide of interest. The term “promoter” is used in its conventional sense and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding an adhesive polypeptide or adhesive fusion polypeptide within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metalothionein.

A promoter can also be a tissue specific promoter, such as a kidney specific promoter, preferably a kidney epithelial cell promoter, which can be natural or synthetic. Examples include, but are not limited to, the CDH 16 promoter, which is mostly kidney specific (it is also expressed in the thyroid) (Igarashi, et al., Am J Physiol., 277(4):F599-610 (1999). Doi: 10.1152/ajprenal.1999.277.4.F599. PMID: 10516285.); the Pax-8 promoter, which is also expressed primarily in the kidney as well as in the thyroid (Dehbi, et al., EMBO J., 15(16):4297-306 (1996) PMID: 8861958); the aquaporin 2 promoter, which drives expression specifically in principal cells of the renal collecting duct (which are the target of Tolvaptan) (Stricklett, et al., Exp Nephrol., 7(1):67-74 (1999). Doi: 10.1159/000020587. PMID: 9892817.), and kidney tubule-specific promoters in association with gene delivery viral vectors (Watanabe, et al., PloS one, vol. 12,3 e0168638 (2017), doi:10.1371/journal.pone.0168638).

In some forms, the promoter is a strong prokaryotic or eukaryotic promoter, such as cytomegalovirus immediate early (CMV-IE) promoter.

ii. Other Expression Control Elements

The disclosed nucleic acids, including vectors, can include additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for an adhesive polypeptide or adhesive fusion polypeptide within an expression cassette of the vector. Enhancer sequences are regulatory DNA sequences that, when bound by transcription factors, enhance the transcription of an associated gene. An enhancer sequence is preferably located upstream of the polynucleotide sequence encoding an adhesive polypeptide or adhesive fusion polypeptide, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human beta-globin polyadenylation signal. Preferably, a polyadenylation signal is a bovine growth hormone (bGH) polyadenylation signal or a SV40 polyadenylation signal.

Any enhancer sequence known to those skilled in the art in view of the present disclosure can be used. For example, an enhancer sequence can be a human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit beta-globin intron, or any combination thereof. Preferably, an enhancer sequence is a composite sequence of three consecutive elements of the untranslated R-U5 domain of HTLV-1 LTR, rabbit beta-globin intron, and a splicing enhancer, which is referred to herein as “a triple enhancer sequence.”

A vector can include a polynucleotide sequence encoding a signal peptide sequence. Preferably, the polynucleotide sequence encoding the signal peptide sequence is located upstream of the polynucleotide sequence encoding an adhesive polypeptide or adhesive fusion polypeptide. Signal peptides typically direct localization of a protein, facilitate secretion of the protein from the cell in which it is produced, and/or improve expression the therapeutic polypeptide when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding an adhesive polypeptide, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., an adhesive polypeptide. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan^{r}), ampicillin resistance gene (Amp^{r}), and tetracycline resistance gene (Tet^{r}), as well as genes conferring resistance to chloramphenicol, bleomycin, spectinomycin, carbenicillin, etc.

An expression vector can include a tag sequence, such as those discussed above.

### 3. Host Cells

Host cells expressing or including an adhesive polypeptide or adhesive fusion polypeptide are also described.

Vectors containing nucleic acids to be expressed can be transferred into host cells. The term “host cell” is intended to include prokaryotic and eukaryotic cells into which a recombinant expression vector can be introduced.

As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell) can be used to, for example, produce the adhesive polypeptide or adhesive fusion polypeptide described herein.

Exemplary host cells include prokaryotic and eukaryotic cells. For example, in some forms host cells include bacteria, yeast, fungi, algae, protozoans, insect cells, mammalian cells, fish cells, worm cells, avian cells and reptile cells.

Any cell lines for expression of isolated recombinant proteins that are known in the art can be used to express the described proteins and fusion proteins thereof.

### 4. Delivery Vehicles

Any of the disclosed compositions including, but not limited to polypeptides and/or nucleic acids, can be delivered to target cells or organisms using a delivery vehicle. The delivery vehicles can be, for example, polymeric particles, inorganic particles, silica particles, liposomes, micelles, multilamellar vesicles, etc.

Delivery vehicles may be microparticles or nanoparticles. Nanoparticles are often utilized for intertissue application, penetration of cells, and certain routes of administration. The nanoparticles may have any desired size for the intended use. The nanoparticles may have any diameter from 10 nm up to about 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In some forms the nanoparticles can have a diameter less than 400 nm, less than 300 nm, or less than 200 nm. The range can be between 50 nm and 300 nm.

Thus, in some forms, the delivery vehicles are nanoscale compositions, for example, 10 nm up to, but not including, about 1 micron. However, it will be appreciated that in some forms, and for some uses, the particles can be smaller, or larger (e.g., microparticles, etc.). Although many of the compositions disclosed herein are referred to as nanoparticle or nanocarrier compositions, it will be appreciated that in some forms and for some uses the carrier can be somewhat larger than nanoparticles. Such compositions can be referred to as microparticulate compositions. For example, a nanocarriers according to the present disclosure may be a microparticle. Microparticles can a diameter between, for example, 0.1 and 100 μm in size.

In some forms, the carrier is a liposome or micelle. Liposomes are spherical vesicles composed of concentric phospholipid bilayers separated by aqueous compartments. Liposomes can adhere to and form a molecular film on cellular surfaces. Structurally, liposomes are lipid vesicles composed of concentric phospholipid bilayers which enclose an aqueous interior (Gregoriadis, et al., Int. J. Pharm., 300, 125-30 2005; Gregoriadis and Ryman, Biochem. J., 124, 58P (1971)). Hydrophobic compounds associate with the lipid phase, while hydrophilic compounds associate with the aqueous phase.

Liposomes include, for example, small unilamellar vesicles (SUVs) formed by a single lipid bilayer, large unilamellar vesicles (LANs), which are vesicles with relatively large particles formed by a single lipid bilayer, and multi-lamellar vesicles (MLVs), which are formed by multiple membrane layers. Thus, the liposomes can have either one or several aqueous compartments delineated by either one (unilamellar) or several (multilamellar) phospholipid bilayers (Sapra, et al., Curr. Drug Deliv., 2, 369-81 (2005)). Multilamellar liposomes have more lipid bilayers for hydrophobic therapeutic agents to associate with. Thus, potentially greater amounts of therapeutic agent are available within the liposome to reach the target cell.

Liposomes have the ability to form a molecular film on cell and tissue surfaces. Clinical studies have proven the efficacy of liposomes as a topical healing agent (Dausch, et al., Klin Monatsbl Augenheilkd 223, 974-83 (2006); Lee, et al., Kin Monatsbl Augenheilkd 221, 825-36 (2004)). Liposomes have also been used in ophthalmology to ameliorate keratitis, corneal transplant rejection, uveitis, endophthalmitis, and proliferative vitreoretinopathy (Ebrahim, et al., 2005; Li, et al., 2007).

### B. Adhesive Polypeptide Glues and Coatings

In some forms the adhesive polypeptide e.g., that includes all or part of the adhesin Biofilm-Associated Protein 1 (Bap1) from V. cholerae, or a variant thereof is formulated into an adhesive composition, such as an adhesive glue.

Typically, adhesive polypeptide compositions include one or more suitable solvents, fillers, mixers or excipients for combining with an adhesive polypeptide or fusion polypeptide thereof to provide a formulation such as a glue.

Therefore, in some forms, composition of adhesive glues include:
(i) an adhesive polypeptide e.g., including or having an amino acid sequence of SEQ ID NO:1 or 4, or a functional fragment, variant include homologous, or fusion polypeptide thereof; and
(ii) an excipient.

Typically, the adhesive polypeptide is suspended or mixed within the excipient. In some forms, the adhesive polypeptide component includes the majority (i.e., more than 50%) of the total weight of the formulation. In other forms, the adhesive polypeptide or a functional fragment, variant, or fusion polypeptide thereof includes 50% or less of the total weight of the formulation. In some forms, the adhesive polypeptide or a functional fragment, variant, or fusion polypeptide thereof imparts between 0.1% and 99.9% by weight of the glue or coating. In other forms, the adhesive polypeptide or a functional fragment, variant, or fusion polypeptide thereof imparts between 1% and 50% by weight of the glue or coating.

Exemplary glues and/or coatings include the form of a liquid, an oil, a gel, an emulsion, a cream, an aerosol, a powder and a foam. Therefore, in some forms, the adhesive polypeptide is formulated with one or more excipients to provide a glue in the form of a liquid, an oil, a gel, an emulsion, a cream, an aerosol, a powder and a foam.

Excipients can include one or more preservatives or other active agents to maintain, enhance or otherwise affect the adhesive polypeptides. Exemplary excipients can be toxic or non-toxic, and they can be biodegradable or non-biodegradable. In some forms, formulations of adhesive peptides include a filler, a surfactant, or an oxidant. Exemplary fillers include collagen, hyaluronic acid, condroitan sulfate, elastine, laminin, caseine, hydroxyapatite, albumin, fibronectin, and hybrin. Coating materials including adhesive polypeptide or a functional fragment, variant, or fusion polypeptide thereof are also provided.

In some forms adhesive peptide glues are developed for use in aqueous solutions. In some forms, the aqueous solutions include ions and small molecules including magnesium, sodium, potassium, calcium, sulfate, chloride, sugars, phosphate, lipids, detergents, and chaotropes. In some forms, the aqueous solutions include organic solvents, such as ethanol, methanol, acetone, toluene, and ammonia.

Any of the glues and coating materials can be packaged into a container. Typically, the container includes means for extrusion of the glue or coating material. In some forms, the container is formulated from a material that does not adhere to the glue or coating. Therefore, in some forms, the glue or coating material does not adhere to the inside of the container.

C. Compositions Coated with or Including Adhesive Polypeptides

Compositions including the adhesive polypeptide or a functional fragment, variant, or fusion polypeptide thereof in the form of a glue or an adhesive patch or strip coated or painted onto a substrate are also provided.

Exemplary substrates include biotic and abiotic surfaces. In some forms the substrate is or includes a material selected from metal, stone, plastic, glass, silica, concrete, paint, carbon, rubber, ceramic, lipids, and polymers, or combinations thereof. Therefore, in some forms, the compositions include an adhesive polypeptide, e.g., having or including an amino acids sequence of SEQ ID NO:1 or 4, or a functional fragment, variant including homologous thereof, or fusion polypeptide thereof in the form of a glue or an adhesive patch or strip coated or pained onto a substrate selected from metal, stone, plastic, glass, silica, concrete, paint, carbon, rubber, ceramic, lipids, and polymers, or combinations thereof. In some forms, the substrate is a bead, such as a metal, glass, silicate, carbon, polymeric bead, or a biotic surface such as a cell (i.e., plasma membrane).

In some forms, the substrate is or includes one or more hydrophilic polymers.

In some forms, the substrate is or includes one or more hydrophobic polymers. In some forms, the substrate is a particle. In other forms, the substrate is a surface on a composition that is not a particle.

In some forms, the substrate is or contains an aliphatic polyester. In some forms, the substrate is a particle containing poly(lactic acid), poly(glycolic acid), or poly(lactic acid-co-glycolic acid).

The substrate can contain one or more biodegradable polymers. Biodegradable polymers include polymers that are insoluble or sparingly soluble in water that are converted chemically or enzymatically in the body into water-soluble materials. In some forms, the substrate contains biodegradable polymers including soluble polymers crosslinked by hydolyzable cross-linking groups to render the crosslinked polymer insoluble or sparingly soluble in water.

In some forms, the substrate is or contains one or more amphiphilic polymers. Amphiphilic polymers can be polymers containing a hydrophobic polymer block and a hydrophilic polymer block. The hydrophobic polymer block can contain one or more of the hydrophobic polymers above or a derivative or copolymer thereof. The hydrophilic polymer block can contain one or more of the hydrophilic polymers above or a derivative or copolymer thereof. In some forms the amphiphilic polymer is a di-block polymer containing a hydrophobic end formed from a hydrophobic polymer and a hydrophilic end formed of a hydrophilic polymer. In some forms, a moiety can be attached to the hydrophobic end, to the hydrophilic end, or both.

In some forms, the substrate is or contains a first amphiphilic polymer having a hydrophobic polymer block, a hydrophilic polymer block, and targeting moiety conjugated to the hydrophilic polymer block; and a second amphiphilic polymer having a hydrophobic polymer block and a hydrophilic polymer block but without the targeting moiety. The hydrophobic polymer block of the first amphiphilic polymer and the hydrophobic polymer block of the second amphiphilic polymer may be the same or different. Likewise, the hydrophilic polymer block of the first amphiphilic polymer and the hydrophilic polymer block of the second amphiphilic polymer may be the same or different.

In some forms, the substrate is or contains a biodegradable polyesters or polyanhydrides such as poly(lactic acid), poly(glycolic acid), and poly(lactic-co-glycolic acid).

The substrate can also contain one or more polymer conjugates containing end-to-end linkages between the polymer and a targeting moiety or a detectable label. For example, a modified polymer can be a PLGA-PEG-peptide block polymer.

The substrate can contain one or a mixture of two or more polymers. When the substrate is a particle, the particles may contain other entities such as stabilizers, surfactants, or lipids. The particles may contain a first polymer having a targeting moiety and a second polymer not having the targeting moiety. By adjusting the ratio of the targeted and non-targeted polymers, the density of the targeting moiety on the exterior of the particle can be adjusted.

The substrate can contain an amphiphilic polymer having a hydrophobic end, a hydrophilic end, and a targeting moiety attached to the hydrophilic end. In some forms the amphiphilic macromolecule is a block copolymer having a hydrophobic polymer block, a hydrophilic polymer block covalently coupled to the hydrophobic polymer block, and a targeting moiety covalently coupled to the hydrophilic polymer block. For example, the amphiphilic polymer can have a conjugate having the structure A-B-X where A is a hydrophobic molecule or hydrophobic polymer, B is a hydrophilic molecule or hydrophilic polymer, and X is a targeting moiety. Exemplary amphiphilic polymers include those where A is a hydrophobic biodegradable polymer, B is PEG, and X is a targeting moiety that targets, binds.

In some forms, the substrate is or contains a first amphiphilic polymer having the structure A-B-X as described above and a second amphiphilic polymer having the structure A-B, where A and B in the second amphiphilic macromolecule are chosen independently from the A and B in the first amphiphilic macromolecule, although they may be the same.

In some embodiments, the substrate is an abiotic surface. In other forms, compositions include adhesive polypeptides coated onto a silica substrate, such as a silica bead. In other forms, compositions include adhesive polypeptides coated onto a polystyrene substrate, such as a latex bead or particle.

In some forms, the substrate is or includes Polytetrafluoroethylene (PTFE). In some forms, the substrate is or includes Polydimethylsiloxane (PDMS).

In some forms, when compositions include a glue or coating in contact with a substrate, the detachment force required to remove the adhered glue or coating corresponds to the maximum adhesive strength of the glue F_max. F_max is related to W, the work of adhesion, through F_max=3/2πRW, in which R is the radius of a bead coated with the glue.

In an exemplary form, the peak value of F_max is between about 1 nN and about 100 nN, inclusive, such as between about 2 nN and about 10 nN, inclusive. In a particular form, the peak value of F_max is 2.82 nN, equivalent to a W of 0.24 mJ/m².

In some embodiments, the surface is biotic surface. The results in the Examples below illustrate that the biofilm-derived peptides can adhere to a wide range of lipid compositions, and the various parts of the sequence play complementary roles in lipid adhesion. This is some embodiments, the substate is a lipid(s), and lipid-containing surfaces such as plasma membranes. These results indicate that the peptide not only binds to abiotic surfaces but also to important biotic surfaces such as those in humans, making it suitable for biomedical applications, e.g., in vivo.

### III. Methods of Making

### A. Methods for Producing Adhesive Polypeptides

Isolated adhesive polypeptides, e.g., having or including an amino acid sequence of SEQ ID NO:1 or 4, or functional fragments, variants including homologues, and fusion polypeptides thereof can be obtained by, for example, chemical synthesis or by recombinant production in a host cell.

To recombinantly produce adhesive polypeptides, a nucleic acid containing a nucleotide sequence encoding the polypeptide, typically a vector such as those discussed above, can be used to transform, transduce, or transfect a bacterial or eukaryotic host cell (e.g., an insect, yeast, or mammalian cell). In general, nucleic acid constructs include a regulatory sequence operably linked to a nucleotide sequence encoding the adhesive polypeptides. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well know in the art include, for example, Escherichia coli strains such as BL-21, and cultured mammalian cells such as CHO cells.

In eukaryotic host cells, several viral-based expression systems can be utilized to express the provided adhesive polypeptides. Viral based expression systems are well known in the art and include, but are not limited to, baculoviral, SV40, retroviral, or vaccinia based viral vectors.

Mammalian cell lines that stably express adhesive polypeptides can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of variant costimulatory polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, the polypeptide can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Adhesive polypeptides can be isolated using, for example, chromatographic methods such as DEAE ion exchange, gel filtration, and hydroxylapatite chromatography. For example, the 57-aa sequence of SEQ ID NO:1 or 4, or a functional fragment, variant including homologues, or a fusion polypeptide thereof in a cell culture supernatant or a cytoplasmic extract can be isolated using a protein G column.

As discussed above, in some forms, the adhesive polypeptide is “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify the polypeptides.

### 1. Production of Adhesive Proteins Alone

As discussed in the Examples, in some forms, the adhesive polypeptide is expressed alone, for example, by over-expression in a prokaryotic expression system. Exemplary expression techniques to express the adhesive polypeptide include using E. coli expression constructs of the adhesive polypeptide. In some forms, the adhesive polypeptide is fused to a polyhistidine tag, and/or with a fused pelB secretion tag (and his tag) which is a signal that directs proteins to the periplasm. In some forms, a pelB leader is included on the original GFP_{UV} fusion construct to facilitate periplasmic targeting and help with solubility.

Additionally, or alternatively, adhesive polypeptide can be prepared as a protein conjugate with one or more functional elements (e.g., protein transduction domains, fusogenic peptides, targeting molecules, tags, etc. chemically conjugated thereto. Methods for attaching peptides, small molecules, and other compounds to polypeptides are well known in the art and can include use of bifunctional chemical linkers such as N-succinimidyl (4-iodoacetyl)-aminobenzoate; sulfosuccinimidyl(4-iodoacetyl)-aminobenzoate; 4-succinimidyl-oxycarbonyl-V-(2-pyridyldithio) toluene; sulfosuccinimidyl-6-[alpha-methyl-V-(pyridyldithiol)-toluami-do]hexanoate; N-succinimidyl-3-(-2-pyridyldithio)-proprionate; succinimidyl-6-[3 (-(-2-pyridyldithio)-proprionamido]hexanoate; sulfosuccinimidyl-6-[3 (-(-2-pyridyldithio)-propionamido]hexanoate; 3-(2-pyridyldithio)-propionyl hydrazide, Ellman's reagent, dichlorotriazinic acid, S-(2-thiopyridyl)-L-cysteine, and the like. Further bifunctional linking molecules are discussed in, for example, U.S. Pat. Nos. 5,349,066, 5,618,528, 4,569,789, 4,952,394, and 5,137,877. The linker can be cleavable or noncleavable. Highly stable linkers can reduce the amount of payload that falls off in circulation, thus improving the safety profile, and ensuring that more of the payload arrives at the target cell. Linkers can be based on chemical motifs including disulfides, hydrazones or peptides (cleavable), or thioethers (noncleavable) and control the distribution and delivery of the active agent to the target cell. Cleavable and noncleavable types of linkers have been proven to be safe in preclinical and clinical trials (see, e.g., Brentuximab vedotin which includes an enzyme-sensitive linker cleavable by cathepsin; and Trastuzumab emtansine, which includes a stable, non-cleavable linker). In particular forms, the linker is a peptide linker cleavable by Edman degradation (Bachor, et al., Molecular diversity, 17 (3): 605-11 (2013)).

### 2. Production of Adhesive Proteins as Fusions

When the adhesive polypeptide, e.g., the 57-aa sequence of SEQ ID NO:1 or 4, or a functional fragment, variant, or fusion polypeptide thereof includes a heterologous sequence or sequences it is most typically prepared as a fusion protein. Fusion proteins or chimeric proteins are proteins created through the joining of two or more nucleic acid sequences that originally coded for separate polypeptides. Translation of this fusion gene results in a single polypeptide with functional properties derived from each of the original polypeptides. Recombinant fusion proteins are created artificially by recombinant DNA technology. e.g., as discussed above.

In some forms, the adhesive domain, e.g., the 57-aa sequence of SEQ ID NO:1 or 4, or a functional fragment, or variant, or a fusion polypeptide thereof is attached to the end of a fragment of a stable, well-expressed carrier protein, such as FrhA. The larger protein improves the solubility of the small peptide. In some forms, the expression construct includes an additional proteolytic site between FrhA and the adhesive domain to cleave off the adhesive domain.

In some forms, the adhesive domain, e.g., the 57-aa sequence of SEQ ID NO:1 or 4, or a functional fragment, or variant, or a fusion polypeptide thereof is expressed together with Vibrio cholerae cytolysin (VCC), a pore-forming toxin that is expressed in E. coli. Pore-forming toxins are secreted in a water-soluble state, but then bind to membranes and form a transmembrane channel. So, an important feature of their function is that in the water-soluble state, they can shield a hydrophobic loop from the solvent and keep it soluble in solution. In some forms, this hydrophobic loop is replaced with the adhesive domain, so that VCC can act as a molecular chaperone to produce soluble material. In some forms, the methods also include engineering in protease sites to release the peptide following purification. In other forms one or more other pore-forming toxins are expressed together with the adhesive polypeptide. In some forms, the methods include contacting the expressed proteins with detergents or other small molecules that trigger correct folding and assembly of the adhesive polypeptides in solution. The methods preferably express a polypeptide including an adhesive domain, e.g., the 57-aa sequence of SEQ ID NO:1 or 4, or a functional fragment, variant, or fusion polypeptide thereof in a soluble form that can be triggered to attach to a surface by exposure of the adhesive domain loop. Therefore, in some forms the methods provide a triggerable glue that might have interesting applications in industrial or biomedical settings.

In some forms, the polypeptide including an adhesive domain, e.g., the 57-aa sequence of SEQ ID NO:1 or 4, or a functional fragment, variant, or fusion polypeptide thereof is expressed in the presence of, and/or associated with one or more additional proteins or molecules that are known in the art to function to keep insoluble sequences in solution. Exemplary proteins or molecules that assist to keep proteins in solution include pore-forming toxins, molecular chaperones and other stable, soluble proteins, such as albumen.

In some forms, the methods tag the polypeptide including an adhesive domain, e.g., the 57-aa sequence of SEQ ID NO: 1 or 4, or a functional fragment, variant, or fusion polypeptide thereof with one or more targeting domain or moiety that targets other proteins or nucleic acids to the site of the glue. Therefore, in some forms, the methods express a tagged glue that is targeted to a specific site or location or cell type in vivo, and then target a second protein or molecule to the glue.

### B. Methods for Producing Isolated Nucleic Acid Molecules

Isolated nucleic acid molecules encoding polypeptides such including an adhesive domain, e.g., the 57-aa sequence of SEQ ID NO:1 or 4, or a functional fragment, variant, or fusion polypeptide thereof can be produced by standard techniques, including, without limitation, common molecular cloning and chemical nucleic acid synthesis techniques. For example, polymerase chain reaction (PCR) techniques can be used to obtain an isolated nucleic acid encoding a variant costimulatory polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Polypeptide encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

### IV. Pharmaceutical Compositions

The disclosed compositions alone or in a delivery vehicle can be formulated with appropriate pharmaceutically acceptable carriers into pharmaceutical compositions for administration to an individual in need thereof. The formulations can be administered enterally (e.g., oral) or parenterally (e.g., by injection or infusion).

The disclosed compositions can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

The peptides may be in solution, emulsions, or suspension (for example, incorporated into microparticles, liposomes, or cells). In some embodiments, an appropriate amount of a pharmaceutically-acceptable salt is used in the formulation to render the formulation isotonic. Examples of pharmaceutically-acceptable carriers include, but are not limited to, saline, Ringer's solution and dextrose solution. The pH of the solution is preferably from about 5 to about 8, and more preferably from about 7 to about 7.5. Pharmaceutical compositions may include carriers, thickeners, diluents, buffers, preservatives, detergents, and surface active agents. Further carriers include sustained release preparations such as semi-permeable matrices of solid hydrophobic polymers containing the peptides, which matrices are in the form of shaped particles, e.g., films, liposomes or microparticles. It will be apparent to those persons skilled in the art that certain carriers may be more preferable depending upon, for instance, the route of administration and concentration of composition being administered. Pharmaceutical compositions may also include one or more active ingredients such as antimicrobial agents, anti-inflammatory agents, and anesthetics. Preparations for parenteral administration include sterile aqueous or non-aqueous solutions, suspensions, and emulsions. Parenteral vehicles include sodium chloride solution, Ringer's dextrose, dextrose and sodium chloride, lactated Ringer's, or fixed oils. Preservatives and other additives may also be present such as, for example, antimicrobials, anti-oxidants, chelating agents, and inert gases.

To aid dissolution of peptides into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios. Additives which potentially enhance uptake of peptides are for instance the fatty acids oleic acid, linoleic acid and linolenic acid.

In some embodiments, where the peptide is aggregated to use in a water-soluble form, an additive and/or non-aqueous solvent can be used. For in vivo application these are preferably compatible with medical uses. Examples include, but are not limited to, alcohols, DMSO, urea, guanidinium hydrochloride, detergents, amphiphiles, etc.

### V. Methods of Use

In a serendipitous discovery while studying how Vibrio cholerae (Vc) biofilms adhere to surfaces, it was discovered that a short protein sequence made of 57-amino acids that is majorly responsible for Vc adhesion to various abiotic surfaces, including glass and plastics (see FIGS. 8A-8C). The peptide also adheres to biotic surfaces such lipids and lipid-containing compositions such as plasma membranes.

Through further investigation of the purified peptide and variations of the original sequence, it was established that this is an effective, generic, and readily manipulatable peptide for applications involving underwater adhesion. Because of its bacterial origin and the absence of posttranslational modifications, it is believed it can be mass produce through fermentation, paving the way for scaling up its production for industrial or biomedical use. This sequence is also more stable under various pH conditions and insensitive to oxidation.

These findings identify a polypeptide sequence derived from a pathogen that are useful for industrial applications. The sequence is not associated with Vc pathogenicity and is also present in other Vibrio species that are not human pathogens so it is safe. The simplicity in manipulating peptide sequences in bacteria allows rapid testing and screening for optimal sequences for particular applications (i.e., a particular surface to which a desired protein is targeted). Also, the sequence can be easily integrated into other recombinant proteins to direct the protein to adhere to surfaces. It was also found that the adhesion function of the peptide is unaffected by the properties of the surface including hydrophobicity and charge, which is advantageous for wide range of applications.

### A. Methods of Adhesion Under Aqueous Conditions

Methods and systems for using the described polypeptides including an adhesive domain, e.g., having or including the 57-aa sequence of SEQ ID NO:1 or 4, or a functional fragment, variant, or fusion polypeptide thereof to adhere to one or more substrates, such as abiotic and/or biotic surfaces are provided. Typically, the methods apply or include an effective amount of the adhesive polypeptide to one or more substrates under aqueous conditions, such as under water. Exemplary abiotic surfaces include substrates formulated from a material selected from metal, stone, plastic, glass, silica, concrete, paint, carbon, rubber, ceramic, and polymer fabric, or combinations thereof.

Therefore, methods of adhering two or more compositions together under aqueous conditions are provided. Typically, the methods include:
(i) contacting a first composition with an effective amount of adhesive polypeptide, e.g., including the 57-aa sequence of SEQ ID NO:1 or 4, or a functional fragment, variant, or fusion polypeptide thereof, or with a glue or coating including the adhesive polypeptide, to form a first adhesive composition including an adhesive layer; and
(ii) contacting a second composition with the first adhesive composition. Typically, the contacting includes interaction between the second composition and the adhesive layer, sufficient for the first adhesive composition and second composition to adhere together.

In some forms, the contacting in step (i), or step (ii), or in both steps (i) and (ii) occurs in an aqueous environment.

In some forms the adhesive layer includes the adhesive polypeptide or the glue in an amount between about 0.01 μg/cm² and about 100 μg/cm², inclusive.

The methods can be carried out in vitro or in vivo, and can be applied to adhere two materials together, via an adhesive surface including the adhesive polypeptide. The methods can adhere two abiotic materials, two biotic materials, or a biotic material and an abiotic material. Therefore, methods for adhering a biotic surface to an abiotic surface are provided. Typically, the methods include coating or contacting a biotic and/or abiotic material with an effective amount of the adhesive polypeptide e.g., including the 57-aa sequence of SEQ ID NO:1 or 4, or a functional fragment, variant, or fusion polypeptide thereof to create an adhesive surface on the material. The methods then contact an abiotic material with a biotic material in the region of the adhesive surface, to adhere the biotic and abiotic materials together.

### 1. In Vivo Applications

The aqueous environment can be inside or outside of the body of a subject. For example, in some forms, the aqueous environment is within the body of a human. Therefore, in some forms the methods include administering the adhesive polypeptide e.g., including the 57-aa sequence of SEQ ID NO:1 or 4, or a functional fragment, variant, or fusion polypeptide thereof in vivo. Typically, the administration includes application onto or into a tissue, cell, or bodily fluid.

a. Tissue Glue

In some forms, the application includes administration of an adhesive polypeptide or fusion protein thereof into or onto a biotic surface in vivo, or in vitro, for example, administration onto a tissue or organ or cells ex vivo. The results presented below show that peptides can bind to lipids and lipid-containing biotic surfaces such as plasma membranes.

Thus, the peptides can be used with, or without, the addition of a further tissue-binding moiety. For example, in some in vivo applications for binding to biotic surfaces, the adhesive peptides or fusion polypeptides thereof include at least one tissue or cell-binding moiety for binding the adhesive protein to an abiotic surface.

In some forms, following administration in vitro or ex vivo, the methods include administering the composition(s) and/or devices including the adhesive polypeptide e.g., including the 57-aa sequence of SEQ ID NO:1 or 4, or a functional fragment, variant, or fusion polypeptide thereof into or onto the body of a subject. For example, in some forms, the methods administer an organ or tissue or blood into or onto the body of a subject following administration of an adhesive domain or fusion peptide thereof onto or into the organ or tissue or blood. In some forms, the adhesive polypeptide e.g., including the 57-aa sequence of SEQ ID NO:1 or 4, or a functional fragment, variant, or fusion polypeptide thereof is used to adhere one or more first tissues or organs or other biological material to a second or further tissue or organ or other biological material. In some forms, the tissue is skin. For example, in some forms, a composition including the adhesive polypeptide, e.g., including the 57-aa sequence of SEQ ID NO:1 or 4, or a functional fragment, variant, or fusion polypeptide thereof is used to glue a wound or tear in the skin together, for example to stop or inhibit bleeding or movement of fluids from a wound or tear in the skin. In other forms, a composition including the adhesive polypeptide, e.g., including the 57-aa sequence of SEQ ID NO:1 or 4, or a functional fragment, variant, or fusion polypeptide thereof is used to attach in the mouth or nose, for example, to coat or bind to a tooth, gum, tonsil, lip or other tissue in or around the oral cavity.

b. Components of Prosthetic Devices

In some forms, the application includes administration into or onto an abiotic surface in vivo, or in vitro, for example, administration onto or into a prosthetic device. The administration can be in vitro or in vivo prior to administration into or onto the device. In some forms, following administration to a prosthetic device in vitro or ex vivo, the methods include administering the prosthesis including the adhesive polypeptide e.g., including the 57-aa sequence of SEQ ID NO:1 or 4, or a functional fragment, variant, or fusion polypeptide thereof into or onto the body of a subject. For example, in some forms, the methods administer a stent, a vascular graft, a pacemaker, a prosthetic valve, a prosthetic joint, a catheter, a colostomy device, or other prosthesis into or onto the body of a subject following administration of an adhesive domain or fusion peptide thereof onto or into the device. In some forms, the adhesive material is used to create an adhesive force for a specific period of time, for example, to allow for tissue growth/regrowth, would healing, tissue remodeling, etc.

i. Stents

In some forms, the adhesive polypeptide e.g., including the 57-aa sequence of SEQ ID NO:1 or 4, or a functional fragment, variant, or fusion peptide thereof is used to coat or otherwise bind to a stent.

Many stents are commercially available or otherwise know in the art. Stents can be formed, i.e., etched or cut, from a thin tube of suitable material, or from a thin plate of suitable material and rolled into a tube.

Suitable materials for the stent include but are not limited to stainless steel, iridium, platinum, gold, tungsten, tantalum, palladium, silver, niobium, zirconium, aluminum, copper, indium, ruthenium, molybdenum, niobium, tin, cobalt, nickel, zinc, iron, gallium, manganese, chromium, titanium, aluminum, vanadium, and carbon, as well as combinations, alloys, and/or laminations thereof. For example, the stent may be formed from a cobalt alloy, such as L605 or MP35N®, Nitinol (nickel-titanium shape memory alloy), ABI (palladium-silver alloy), Elgiloy® (cobalt-chromium-nickel alloy), etc. It is also contemplated that the stent may be formed from two or more materials that are laminated together, such as tantalum that is laminated with MP35N®. The stents may also be formed from wires having concentric layers of different metals, alloys, or other materials. Forms of the stent may also be formed from hollow tubes, or tubes that have been filled with other materials. The aforementioned materials and laminations are intended to be examples and are not intended to be limiting in any way.

Stents can be drug-eluting stents. Various drug eluting stents that simultaneously deliver a therapeutic substance to the treatment site while providing artificial radial support to the wall tissue are known in the art. Endo-luminal devices including stents can be coated on their outer surfaces with a substance such as a drug releasing agent, growth factor, antibody, or the like. Stents have also been developed having a hollow tubular structure with holes or ports cut through the sidewall to allow drug elution from a central lumen. Although the hollow nature of the stent allows the central lumen to be loaded with a drug solution that is delivered via the ports or holes in the sidewall of the stent, the hollow tubular structure may not have suitable mechanical strength to provide adequate scaffolding in the vessel. Therefore, in some forms, the adhesive polypeptide e.g., including the 57-aa sequence of SEQ ID NO:1 or 4, or a functional fragment, variant, or fusion peptide thereof is used to coat or otherwise bind to a stent.

ii. Catheters

In some forms, the adhesive polypeptide e.g., including the 57-aa sequence of SEQ ID NO:1 or 4, or a functional fragment, variant, or fusion peptide thereof is used to coat or otherwise bind to a catheter.

Many catheters are commercially available or otherwise know in the art. catheters can be formed, i.e., etched or cut, from a thin tube of suitable material, or from a thin plate of suitable material and rolled into a tube.

iii. Bone Repair Matrices

In some forms, the adhesive polypeptide e.g., including the 57-aa sequence of SEQ ID NO:1 or 4, or a functional fragment, variant, or fusion peptide thereof is used to coat or otherwise bind to a device for bone repair and regrowth.

Many devices for the repair and regrowth of bones are commercially available or otherwise know in the art. Exemplary devices include tissue grafts for the ingrowth of bone and collagen cells. The devices can be formed, i.e., fabricated or woven, from a mixture of flexible or semi-rigid fibers that can be biodegradable or non-biodegradable.

iv. Dental Devices

In some forms, the adhesive polypeptide e.g., including the 57-aa sequence of SEQ ID NO:1 or 4, or a functional fragment, variant, or fusion peptide thereof is used to coat or otherwise bind to a dental device, e.g., for use in the mouth or throat. In some forms, the material is used to adhere a prosthetic tooth into a socket, to other teeth and/or to the underlying tissue.

Many dental prosthetic devices and fillers for the repair, regrowth or replacement of teeth or oral tissue are commercially available or otherwise know in the art. Exemplary devices include prosthetic teeth and filling agents.

### 2. Underwater Applications

In other forms, the methods are carried out outside of the body, for example, to adhere two materials together in an aqueous environment, via an adhesive surface including the adhesive polypeptide. The methods can adhere two abiotic materials, two biotic materials, or a biotic material and an abiotic material together underwater. Therefore, methods for adhering two or more surfaces underwater are provided. Typically, the methods include coating or contacting a first material with an effective amount of the adhesive polypeptide e.g., including the 57-aa sequence of SEQ ID NO:1 or 4, or a functional fragment, variant, or fusion polypeptide thereof to create an adhesive surface on the first material. The contacting can be carried out in an aqueous environment such as underwater, or outside of aqueous environment, e.g., prior to submersion underwater. The methods then contact the first material with a second material in the region of the adhesive surface, to adhere the first and second abiotic materials together. The adhesion can occur underwater, or outside of aqueous environment, prior to submersion underwater. Exemplary underwater environments include salt water or freshwater, such as an ocean or sea, or a lake, river, loch, reservoir or other body of water.

### 3. Flocculation

The experiments below show the disclosed peptides tend to aggregate in aqueous solution together with suspended particles. The tendency of the peptides to both aggregate on their own and to adsorb on particles (exemplified in the experiments with polystyrene) renders it a good flocculant for applications such as wastewater treatment and chemical purification, in which particles in aqueous solutions need to be removed or recycled. Such methods can include contacting the aqueous solution with an effective amount of a disclosed peptide, waiting a sufficient time for the peptide and target material to co-aggregate, and removing the peptide-bound target material. Such removal can be by or assisted by any suitable means, e.g., gravity, centrifugation, affinity binding, filtration, etc., and combinations thereof Target materials include but are not limited to: soil colloids, emulsion droplets, clay particles, plastic contaminant, algae cells during an unwanted algal bloom, chitin particles in oceanic, particulate aggregates during chemical synthesis, etc.

The disclosure can be further understood by reference to the following paragraphs:

1. An adhesive polypeptide including an adhesive domain including an amino acid sequence of SEQ ID NO:1 or 4, or a functional fragment or variant thereof, and does not include 100% sequence identity to the entire amino acid sequence of SEQ ID NO:2 or 6, wherein the polypeptide adheres to a surface under aqueous conditions.

2. The adhesive polypeptide of paragraph 1, further including one or more additional heterologous amino acid sequences.

3. The adhesive polypeptide of paragraphs 1 or 2, wherein the polypeptide is complexed to, complexed with, or is otherwise associated with one or more additional heterologous molecule(s).

4. The adhesive polypeptide of paragraph 2 or 3, wherein the heterologous molecule is selected from the group including an amino acid, a protein, a nucleic acid, a carbohydrate, a lipid, a metal, a polymer, a cell, a virion, a small molecule, and a mineral, or combinations thereof.

5. The adhesive polypeptide of any one of paragraphs 2, 3 or 4, wherein the heterologous molecule improves a physicochemical property of the polypeptide selected from the group including solubility, adhesion force, cross-linking, and improvement in protein expression, purification, recovery rate, and biodegradability of the adhesive protein.

6. An adhesive polypeptide including an adhesion domain including at least 70% and less than 100% sequence identity to SEQ ID NO:1 or 4, or functional fragment thereof,
wherein the polypeptide adheres to a surface under aqueous conditions, optionally further including a heterologous amino acid sequence and/or a heterologous molecule;
optionally comprising the amino acid sequence of

(SEQ ID NO: 37)
YLGLEWATATVPYLGVEWATATVSYWFFGWATAQVAYLAPVWAEATIPY
AVPVTLSK,
(SEQ ID NO: 38)
YLGLELKTKTVPLLGVELRTKTVSLWFFGLHTKQVALLAPVLKEKTIPL
AVPVILSK,
or
(SEQ ID NO: 39)
YLGLEWKAKAVPYLGVEWRAKAVSYWFFGWHAKQVAYLAPVWKEKAIPY
AVPVTLSK.

7. The adhesive polypeptide of paragraph 6, wherein the variant or fragment is between 25 and 70 amino acids inclusive, or any subrange or specific integer therebetween.

8. The adhesive polypeptide of any one of paragraphs 1-7, wherein the heterologous amino acid sequence and/or heterologous molecule includes one or more purification tags.

9. The adhesive polypeptide of any one of paragraphs 1-8, wherein the heterologous amino acid sequence and/or heterologous molecule includes a second adhesion domain optionally wherein the second adhesion domain adheres to biotic surfaces.

10. The adhesive polypeptide of any one of paragraphs 1-9, wherein the variant includes at least 75% sequence identity of SEQ ID NO:1 or 4, or a functional fragment thereof.

11. The adhesive polypeptide of any one of paragraphs 1-10, wherein the polypeptide adheres to an abiotic surface in an aqueous environment.

12. The adhesive polypeptide of paragraph 11, wherein the abiotic surface includes a material selected from the group including metal, stone, plastic, glass, silica, concrete, paint, carbon, rubber, ceramic, and polymer fabric, or combinations thereof.

13. A polypeptide having an amino acid sequence that is at least 75 to 99% identical to SEQ ID NO:1 or 4, wherein one or more residues are mutated compared to SEQ ID NO:1 or 4, and wherein the polypeptide does not adhere to a surface
optionally comprising the amino acid sequence of

(SEQ ID NO: 40)
YLGLEAKTKTVPALGVEARTKTVSAWFFGAHTKQVAALAPVAKEKTIPA
AVPVTLSK,
(SEQ ID NO: 41)
YLGLEWKTKTVPYWRTKTVSYWHTKQVAYWKEKTIPYAVPVTLSK,
(SEQ ID NO: 42)
YLGLEWKTKTVPYLGVEWRIKTVSYLGPEWHTKQVAYLAPVWKEKTIPY
AVPVTLSK,
(SEQ ID NO: 43)
WKTKTVPY,
(SEQ ID NO: 44)
LGLEWKTKTVPYLGVEWRTKTVSY,
or
(SEQ ID NO: 45)
VSYWFFGWHTK.

14. The polypeptide of paragraph 13, wherein the mutation(s) includes one or more Lysine to Alanine.

15. A nucleic acid encoding the polypeptide of any one of paragraphs 1-14 and a heterologous nucleic acid sequence.

16. The nucleic acid of paragraph 15, wherein the nucleic acid is RNA or DNA.

17. The nucleic acid of paragraphs 15 or 16, wherein the nucleic acid includes a vector, such as an expression vector.

18. A cell including the nucleic acid of any one of paragraphs 15-17, wherein the nucleic acid sequence is heterologous in the cell.

19. The cell of paragraph 18, wherein the cell expresses a polypeptide including the amino acid sequence of SEQ ID NO:1 or 4.

20. The cell of paragraph 18 or 19, wherein the cell is selected from the group including a bacterium, a protozoan, a plant cell, an insect cell, a mammalian cell, a yeast cell and a fungal cell.

21. A composition including the adhesive polypeptide of any one of paragraphs 1-12, wherein the polypeptide forms an adhesive layer within or on the composition.

22. The composition of paragraph 21, wherein the adhesive layer is in contact with a surface, and wherein the adhesive layer adheres the composition to the surface.

23. The composition of paragraph 22, wherein the surface includes a material selected from the group including metal, stone, plastic, glass, silica, concrete, paint, carbon, rubber, ceramic, and polymers, or combinations thereof.

24. The composition of any one of paragraphs 21-23, wherein the adhesive polypeptide includes between 0.1% and 50% by weight of the total composition.

25. An adhesive glue, including
(i) the adhesive polypeptide of any one of paragraphs 1-12; and
(ii) an excipient,
   wherein the adhesive polypeptide is suspended or mixed within the excipient; and
   wherein the adhesive polypeptide includes between 0.1% and 99.9% by weight of the glue.

26. The glue of paragraph 25, wherein the adhesive polypeptide includes between 1% and 50% by weight of the glue.

27. The glue of paragraph 25 or 26, wherein the glue is in the form of a liquid, a gel, an emulsion, a cream, an aerosol, a powder, or a foam.

28. The glue of any one of paragraphs 25 to 27, wherein the excipient is not an aqueous solution.

29. The glue of any one of paragraphs 25 to 28, wherein the excipient is selected from the groups including a surfactant, an oxidant, and a filler,
optionally wherein the filler is selected from the group including collagen, hyaluronic acid, condroitan sulfate, elastine, laminin, caseine, hydroxyapatite, albumin, fibronectin, and hybrin.

30. A coating material including the glue of any one of paragraphs 25-29.

31. A container including the glue of any one of paragraphs 25-29, or coating material of paragraph 30,
wherein the container includes means for extrusion of the glue or coating material, and
wherein the glue or coating material does not adhere to the inside of the container.

32. A method of adhering two or more compositions together, including
(i) contacting a first composition with the adhesive polypeptide of any one of paragraphs 1-12, or with the glue of any one of paragraphs 25-29, to form a first adhesive composition including an adhesive layer;
(ii) contacting a second composition with the first adhesive composition,
   wherein the contacting includes interaction between the second composition and the adhesive layer, sufficient for the first adhesive composition and second composition to adhere together.

33. The method of paragraph 32, wherein the contacting in step (i), or step (ii), or in both steps (i) and (ii) occurs in an aqueous environment.

34. The method of paragraph 32 or 33, wherein the adhesive layer includes the adhesive polypeptide of any one of paragraphs 1-12, or the glue of any one of paragraphs 25-29 in an amount between about 0.01 μg/cm² and about 100 μg/cm², inclusive.

35. The method of paragraph 34, wherein the aqueous environment is underwater.

36. The method of paragraph 35, wherein the underwater environment is in an ocean or sea, or a lake, river, loch, reservoir or other body of water.

37. The method of any one of paragraphs 32-34, wherein the aqueous environment is in or on the body of a subject.

38. The method of paragraph 37, wherein the subject is a human.

39. A method of removing a target molecule in a solution comprising contacting a solution comprising the target molecule with an effective amount of the adhesive polypeptide of any one of paragraphs 1-12 for an effective amount of time to form an aggregate with the target molecule and removing the aggregate from the solution.

40. The method of paragraph 39, wherein the solution is an aqueous solution, and optionally wherein removing the aggregate comprises allowing the aggregate to settle by gravity and/or centrifugation, and/or optionally capturing the aggregate by affinity binding to a substrate the binds to the polypeptide.

41. The method of paragraphs 39 or 40, wherein removing the aggregate includes gravity settling, centrifugation, affinity binding, filtration, or a combination thereof.

42. The method of any one of paragraphs 39-42, wherein the target material is selected from soil colloids, emulsion droplets, clay particles, plastic contaminant, algae cells, chitin particles, and particulate aggregates.

### EXAMPLES

### Example 1: Vibrio cholerae Uses Two Biofilm-Specific Adhesins with Overlapping but Distinct Functions to Achieve Robust Adhesion to Various Types of Surfaces

Recently, single-cell imaging demonstrated that Bap1 and RbmC also play important roles in defining cell ordering during biofilm development in Vibrio cholerae by generating surface-mediated compression (Yan, et al., Proc. Natl. Acad. Sci. USA 113, e5337-5343 (2016)).

To pinpoint the structural basis underlying the adhesion mechanism, the crystal structures of close-to-full length Bap1 and two individual domains of RbmC were determined (De, et al., PLOS Pathog. 14, e1006841 (2018), Kaus, et al., J. Biol. Chem. 294, 14499-14511 (2019)). In the current study, insights from the prior crystallographic work are combined with functional assays in V. cholerae biofilms to demonstrate the modular nature of Bap1 and RbmC and how V. cholerae uses two biofilm-specific adhesins with overlapping but distinct functions to achieve robust adhesion to various types of surfaces

### Methods

### Bacterial Strains

All V. cholerae strains used in this study were derivatives of the wild-type V. cholerae O1 biovar El Tor strain C6706str2. The rugose strain background harbors a missense mutation in the vpvC gene (vpvC^{W240R}) that elevates intracellular c-di-GMP levels. The rugose strains form robust biofilms and thus allow us to focus on the biochemical mechanisms governing biofilm adhesion rather than mechanisms involving gene regulation. Additional mutations were genetically engineered into this V. cholerae strain using the natural transformation (MuGENT) method.

### Bacterial Growth

All strains were grown overnight in lysogenic broth (LB) at 37° C. with shaking. 1×M9 salts were filter sterilized and supplemented with 2 mM MgSO4 and 100 μM CaCl₂) (abbreviated as M9 medium below). Biofilm growth was generally performed in M9 medium supplemented with 0.5% glucose. For complementation experiments, 100 μg/mL Kanamycin is used.

### Strain Construction

Linear PCR products were constructed using splicing-by-overlap extension (SOE) PCR as previously described and used as transforming DNA (tDNA) in chitin dependent transformation reactions. Briefly, individual V. cholerae colonies were grown in LB media at 30° C. for 6 hours to an OD₆₀₀=0.8-1.0. Cells were washed with Instant Ocean (IO) solution and then incubated on chitin particles suspended in IO for 8-16 hours at 30° C. before the tDNA was added. The cultures were then incubated at 30° C. for an additional 8-16 hours. LB was added to the cultures and incubated at 37° C. for 2 hours before plating on LB agar with the appropriate antibiotic. Multiple deletion mutants were made by co-transformation as previously described.

### Biofilm Adhesion Assay

Overnight cultures of the indicated strains constitutively expressing mNeonGreen were grown from individual colonies at 30° C. with shaking in 1.5 ml LB. 30 μL from each culture was used to inoculate 1.5 mL of M9 medium supplemented with 0.5% glucose and grown at 30° C. with shaking until the OD₆₀₀ is between 0.1 and 0.3. The cultures were then diluted to an OD₆₀₀≅0.001. 100 μL of the regrown culture was aliquoted into the wells of a 96-well plate with a glass bottom (MatTek P96G-1.5-5-F) and incubated at 30° C. for 1 hour. The wells were then washed twice with M9 medium and replaced with M9 medium with 0.5% glucose and 0, 0.2, 0.4, 0.6, 0.8, 1, or 1.2 mg/mL BSA. In the complementation experiment, the growth medium additionally contains 0.2% arabinose for P_{BAD}-bap1 and 2% arabinose for P_{BAD}-rbmC. The lid was secured with a layer of parafilm and the 96-well plate was subsequently incubated at 30° C. for 16-24 hours. Thus-prepared samples were imaged with a spinning disk confocal microscope (Nikon Ti2-E connected to Yokogawa W1) using a 60× water objective (numerical aperture=1.20) and a 488 nm laser excitation. For each sample, several locations with 3×3 tiles where imaged and captured with a sCMOS camera (Photometrics Prime BSI). The x-y pixel size was 0.22 m and the z-step size was 3 m. The wells were then washed twice with M9 medium and re-imaged at the same locations. All images presented in this study are raw data rendered using the Nikon Elements software.

### NaOH Treatment of Glass Substrates

For a subset of experiments, the surface of the 96-well plate was treated with NaOH to render it more hydrophilic and negatively charged. Briefly, before adding the cell culture, 100 μL of 1M NaOH aqueous solution was added to the wells and incubated at room temperature for 3 hours, after which the wells were washed twice with DI water.

### Quantification of Adhesion Assays

Image analysis was performed with built-in functions of the Nikon Elements software by thresholding each image layer-by-layer and measuring the total binarized area above the threshold in each layer. The binary area for each sample z-slice was then summed to give the total biovolume, and the ratio of the total biovolume after versus before the washing step was calculated.

### In Situ Immunostaining

Overnight cultures of the indicated strains with WT or mutated rbmC or bap1 tagged with 3×FLAG at the C-terminus and constitutively expressing mNeonGreen were grown following the same procedure as described above. The initial incubation time was 10 min or 1 hour when biofilms were to be grown in the presence of asialofetuin or BSA, respectively. The wells were then washed twice with M9 medium; subsequently, 100 μL of M9 medium with 0.5% glucose and 1 mg/ml asialofetuin (Sigma-Aldrich A4781) or 0.5 mg/ml BSA (Sigma-Aldrich A9647) was added to the well. BSA and asialofetuin spontaneously coat the surface under these conditions. Both conditions included 1 μg/mL Anti-FLAG antibody conjugated to Cy3 (Sigma-Aldrich A9594). The lid was secured with a layer of parafilm and incubated at 30° C. for 16-24 hours. Thus-prepared samples were imaged with a spinning disk confocal microscope (Nikon Ti2-E connected to Yokogawa W1) using a 100× oil immersion objective (numerical aperture=1.35) or a 60× water immersion objective (numerical aperture=1.20) and a 488 nm laser excitation to observe the cells and a 561 nm laser excitation to observe protein localization, with the corresponding filters. The images were captured with a sCMOS camera (Photometrics Prime BSI) at a z-step size of 0.5 μm.

### Quantification of Protein Distribution

The in situ staining image stacks were used for quantifying protein distribution using built-in functions of the Nikon Element software. First, background noise in the 561 nm channel was measured by taking images in locations without any biofilms and subtracted from the data. Next, a circular region of interest was manually defined that contains a single biofilm cluster. To be consistent, only biofilms of similar heights (25-30 μm) were included in the analysis. Subsequently, Anti-FLAG-Cy3 signals at the glass surface ±0.5 μm were added and the total signal over the entire biofilm height was integrated; the ratio between the two values was calculated to quantify the ability of the adhesin to localize at the biofilm-glass interface. The sizes of the puncta in the bap1_{ΔVelcro} biofilms were manually measured using built-in tools in Nikon Element Software.

### Crystal Violet Assay

The indicated V. cholerae strains were grown on LB agar plates at 37° C., and individual colonies were picked to inoculate culture tubes with 3 mL LB and glass beads. The cultures were grown at 37° C. with shaking until exponential phase (OD₆₀₀˜0.5). 1×3-inch glass slides were cut into similar sizes, washed with ethanol, and flame sterilized before being inserted into sterile culture tubes containing 1 mL LB. Exponential phase cultures were used to inoculate the cell culture tubes with the glass slides at an OD₆₀₀=0.01 (for example, 20 μL of a culture at OD₆₀₀=0.5 was used to inoculate the tube containing 1.0 mL LB and a glass slide). The cultures were grown statically at 37° C. for 16 hours. One at a time, the glass slides were carefully removed and washed 3 times with DI water, stained with 1.5 mL of a 0.1% crystal violet solution for 10 min, washed 3 times with DI water, and transferred to a fresh tube containing 1.5 mL of 30% acetic acid to dissolve the stain associated with the pellicles. The stained acetic acid solution was then transferred to a 1.5 mL cuvette to measure the OD₅₅₀.

### Contact Angle Measurement

V. cholerae strains were streaked on LB plates containing 1.5% agar and grown at 37° C. overnight. Individual colonies were inoculated into 3 mL of LB liquid medium containing glass beads, and the cultures were grown with shaking at 37° C. to mid-exponential phase (5-6 h). Subsequently, the cells in the cultures were vortexed, the OD₆₀₀ was measured, and the cultures were back diluted to an OD₆₀₀ of 0.5. 50 μL of this inoculum was applied to an agar plate and spread with a sterile glass rod to enable growth of a biofilm covering the entire plate. Plates were incubated at 37° C. for 24 hours to form a continuous bacterial lawn. A strip of biofilm (about 1 cm×4 cm) with the underlying agar was cut out with a razor blade and transferred onto a piece of glass for imaging. To overcome uptake of water by the underlying biofilm/agar, a dynamic sessile drop method was used: Water was slowly added to the surface by a syringe pump, and the advancing contact angle was measured to approximate the equilibrium contact angle. Side views of biofilm-liquid interfaces were recorded with a Nikon camera (D3300) equipped with a macrolens (Sigma). The contact angle was extracted using the Droplet_Analysis plugin in ImageJ.

### Western Blot and Secretion Assay

V. cholerae strains encoding the indicated constructs with a C-terminal 3×FLAG tag were grown in culture tubes containing 3 mL LB and sterile glass beads overnight at 30° C. The next day, cultures were vortexed to break up pellicles and cell clusters and the OD₆₀₀ was measured. 1 mL of cell suspensions were transferred to a sterile 1.5 ml microcentrifuge tube and spun at 18,000×g for 3 minutes. 500 μL of the cell supernatant was transferred to a fresh 1.5 mL microcentrifuge tube and the rest discarded from the pellet. The cell pellets were resuspended to an OD₆₀₀=10 and lysed for 30 minutes using a lysis solution (1× Bugbuster solution, lysozyme (0.1 mg/mL), and benzonase (≥250 units/mL). 30 μL of each cell suspension was combined with 10 μL of 4×SDS PAGE sample buffer (40% Glycerol, 240 mM Tris pH 6.8, 8% SDS, 0.04% Bromophenol Blue, 5% β-mercaptoethanol) and boiled for 10 minutes at 95° C. Samples were run on a 4-15% Mini-PROTEAN TGX gel in 1×SDS PAGE running buffer (25 mM Tris, 192 mM Glycine, 1% SDS, pH 8.3) at 120 V for 70 minutes. The proteins were transferred to a PVDF membrane in 1× Transfer buffer (25 mM Tris, 192 mM Glycine, 10% methanol, pH 8.3) at 100 V for 1 hour. The membranes were incubated in 5% milk in TBST overnight at 4° C. The membranes were washed 3×10 minutes in 1×TBST. The membranes were blotted using antibody (BioLegend 637311) against DYKDDDDK (SEQ ID NO:8) at 0.1 μg/mL in TBST with 3% BSA for 1 hour at room temperature and washed 3×10 minutes with 1×TBST. Blots were developed by incubation with Super Signal PLUS Pico West Chemiluminescent Substrate for 5 minutes and pictures taken using the BioRad Chemidoc-MP. Analysis of sample signal was performed in ImageJ.

E. coli Protein Expression and Purification

GFP_{UV}-tagged proteins were cloned, expressed, and purified in E. coli. New constructs were made by PCR from V. cholerae genomic DNA or previously cloned genes and traditional sticky-end cloning into the GFP_{UV} fusion vector pNGFP-BC. RbmC_{β-propeller} was made using a two-step PCR stitching reaction. Clones containing inserts were confirmed by DNA sequencing. For expression and purification, LB media supplemented with 100 μg/mL carbenicillin was inoculated with overnight cultures grown at 37° C. to an OD₆₀₀ of 0.5-0.6, induced with 1 mM IPTG, and grown at 18° C. overnight. Cells were pelleted at 5,000 rpm in a Sorvall LYNX 6000 centrifuge (F9-6×1000 LEX rotor) for 15 minutes and lysed by passing three times through an Emulsiflex-C5 high-pressure homogenizer (Avestin, Inc.). Lysate was cleared at 18,000 rpm for 30 minutes at 4° C. (F20-12×50 LEX rotor). The resulting supernatant was loaded onto a 5 mL HisTrap Ni-NTA column (GE Healthcare) equilibrated in 1×TBS (20 mM Tris-HCl, pH 7.8, 150 mM NaCl) and washed with 1×TBS containing 40 mM imidazole. Protein was eluted with 15 mL of 1×TBS containing 250 mM imidazole. Protein samples were further purified over a Sepharose S6 10/300 size-exclusion column (GE Healthcare) preequilibrated with 1×TBS. Protein fractions were pooled after assessing purity using an SDS-PAGE gel.

Biofilm Staining with Purified Proteins

V. cholerae biofilms from cells constitutively expressing mScarlet-I were grown as described above. After overnight biofilm growth, the growth media was replaced with 100 μL of M9 media containing 1 μM of purified GFP-tagged Bap1 domain constructs. The samples were incubated for 30 minutes at room temperature. The media containing protein was then removed and replaced with fresh M9 media. The samples were imaged with a spinning disk confocal microscope using a 60× water objective and a 488 nm laser excitation to observe protein localization and a 561 nm laser excitation to observe the biofilm, with the corresponding filters.

### Caco-2 Cell Culturing

Human colonic epithelial Caco-2 cells (ATCC HTB-37) were cultured in flasks containing Dulbecco's Modified Eagle's Medium (DMEM; Gibco) supplemented with 10% (v/v) heat-inactivated fetal bovine serum (FBS-HI; Gibco) at 37° C. in a humidified 5% CO₂ incubator. After 72 h, cells were collected via dissociation using TrypLE Express (Gibco) and pelleted by centrifugation (300 rcf, 3 min, room temperature in 15-mL conical tubes (Corning); then 21,000 rcf, 2 min, room temperature in Eppendorf tubes). Cell pellets were stored at −80° C. prior to further analysis.

Mammalian Cell Staining with Purified Proteins

For Caco-2 cells, a frozen aliquot of Caco-2 cells as prepared above was gently thawed and then added to 1 mL of M9 medium containing 300 nM DAPI and incubated for 5 minutes at room temperature 100 μL of this cell suspension was aliquoted to sterile 1.5 mL microcentrifuge tubes and spun at 10,000×g for 3 minutes. The staining media were removed and replaced with 100 μL of M9 media containing 1 mg/mL BSA and 1 μM of purified GFP-tagged Bap1/RbmC Q-prism domain constructs or GFP alone. The samples were incubated for 30 minutes at room temperature and then the media was replaced with 100 μL fresh M9 medium and transferred to the wells of a 96-well plate. The samples were imaged with a spinning disk confocal microscope using a 60× water objective and a 405 nm laser excitation to observe the Caco-2 cell nuclei and a 488 nm laser excitation to observe protein localization, with the corresponding filters.

For jejunum tissue slices (Novus Biologicals NBP2-30201), prepared slides were deparaffinized according to the manufacturers protocol. Briefly, the slides were dried for 1 hr at 60° C. and then soaked in xylene 5×4 minutes. The slides were then hydrated in 100%, 95%, and 75% ethanol 2×3 minutes and immersed in water for 5 minutes. Staining solutions containing 4 μg/ml FM-4-64, 300 nM DAPI, 1 mg/ml BSA and 1 μM of purified protein in 1×PBS were added to the slides and incubated for 30 minutes at room temperature. Slides were carefully washed twice with 1×PBS. The samples were imaged with a spinning disk confocal microscope using a 60× water objective and a 405 nm laser excitation to observe the intestinal cells' nuclei, a 561 nm laser excitation to observed cell membranes, and a 488 nm laser excitation to observe the protein localization, with the corresponding filters.

### VPS Purification

VPS purification was performed according to a published protocol with several modifications¹⁸. First, a rugose ΔrbmAΔbap1ΔrbmCΔpomA strain was used as the starting strain for easier separation of cells and VPS and to avoid confounding factors due to matrix proteins. This strain was grown in LB at 30° C. overnight. 50 μL of this inoculum was added into 3 mL of LB liquid medium containing glass beads, and the cultures were grown with shaking at 30° C. for 3-3.5 h. 50 μL of this inoculum was applied to an agar plate containing M9 medium with 0.5% glucose and 0.5% casamino acids and shaken with glass beads to enable growth of a biofilm covering the entire plate. Plates were incubated at 30° C. for 2 days to form a continuous bacterial lawn. For each purification batch, 10 plates were used. The biofilms were scraped off the agar plates carefully and resuspended in 1×PBS. Biofilm cells were collected by centrifugation (5,000×g, 4° C., 45 min). The supernatant was clarified with additional centrifugation (8,000×g, 4° C., 45 min) and dialyzed for 2 days against distilled water using a dialysis cassette (10 kDa MWCO) with repeated water changes. The dialyzed sample was lyophilized to prepare crude VPS extract. The crude extract was dissolved in 10 mM Tris buffer at 1.5 mg/mL, treated with DNAse and RNAse (37° C., 24 h), and then Proteinase K (37° C., 48 h), followed by ultracentrifugation at 100,000×g for 1 h to remove lipopolysaccharide. This solution was dialyzed against water for 3 days and lyophilized to provide VPS for the binding assay. For each purification batch, typically 10 mg of VPS was obtained as a white powder after the final lyophilization step. The VPS solutions were heated at 95° C. for 10 mins to denature Proteinase K before use.

### VPS or BSM Binding Assays

Gels were prepared with a final concentration of 10% acrylamide (Bio-Rad) in the running gel and 5% in the stacking gel. The native running buffer contained 25 mM Tris-HCl, pH 8.3, and 192 mM glycine. The native loading buffer was made with 62.5 mM Tris-HCl, pH 6.8, 25% glycerol and 1% bromophenol blue dye. Samples for the gel-shift assay were prepared with 5 g of protein per sample. For the VPS gradient, 0, 0.0625, 0.125, 0.25, 0.50, 1 and 5 μg of VPS was preincubated with the representative protein for 5 min. For the BSM gradient, 0, 0.5, 1, 2, 4, 6, 8, 10 g of BSM was preincubated with RbmC_{M1M2} for 5 min. For the GFP control, the highest amount of VPS or BSM was used. For testing different polysaccharides, 5 μg (highest amount used in the VPS concentration gradients) was preincubated with the Bap1A57aa (5 g) for 5 min. Gel electrophoresis was performed at 85 V for 4 hours in an ice bath. Images were acquired on gels (still encapsulated in glass) with an excitation wavelength of 492 nm and an emission wavelength of 513 nm using a Typhoon FLA 9000 imaging system (GE Healthcare).

### Fluorescent Labeling of RbmC_{M1M2}

Purified RbmC_{M1M2} was fluorescently labeled by primary-amine chemistry using an Alexa Fluor-488 TFP ester reagent (Thermo Scientific A37570). Prior to labeling, purified RbmC_{M1M2} was buffer exchanged into 1× phosphate buffered ½ saline (10 mM Na₂HPO₄, 1.8 mM KH₂PO₄, 2.7 mM KCl, and 75 mM NaCl, pH 8.3). For labeling, 1 mg/mL of RbmC_{M1M2} was used. While stirring, 100 μg of the dye (resuspended in 10 μL DMSO) was added to 600 μL of protein-containing solution and incubated for 1 hour at room temperature. Unreacted dye was removed from labeled proteins by running over a Superose 6 10/300 size exclusion column equilibrated with 1× phosphate buffered ½ saline.

### Phylogeny Analysis

Bap1 or RbnmC protein sequences from V. cholerae were used as a query to BLAST against each of the 20 genomes of Vibrio species. BLAST hits with an E-value lower than 1e⁻¹⁵ and alignment coverage (fraction of overlapping positions over the sequence alignment length) higher than 80% of the query were recorded as significant hits—the query gene is recovered in the target genome. Blast hits with an E value higher than 1e⁻¹⁵ but lower than 1e⁻⁵ were recorded as potential hits of the query gene with low conservation. A protein sequence alignment containing significant hits and the query sequence was manually examined to confirm the recovery of the query gene. The Bap1 protein with a 6 aa insertion in V. cholerae O16 str. 877-163 was recovered from a BLAST search against NCBI nr database GenBank accession No. KQA25168.1.

### Statistics

Error bars correspond to standard deviations from measurements taken from distinct samples. Standard t-tests were used to compare treatment groups and are indicated in each figure legend. Tests were always two-tailed, unpaired, and used Welch's correction, as demanded by the details of the experimental design. All statistical analyses were performed using GraphPad Prism software.

### Results

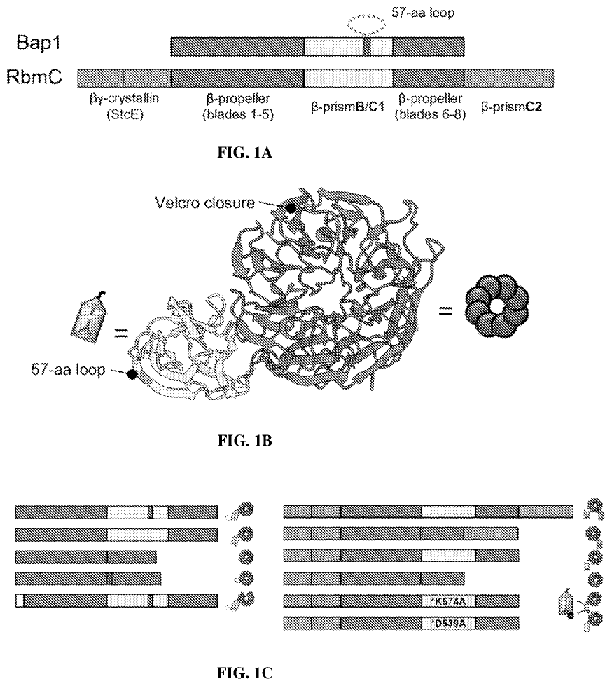

A simple representation of the constituent domains within the protein sequences of Bap1 and RbmC is shown in FIG. 1A. A schematic representation of the crystal structure of Bap1 without the 57-aa loop and the corresponding cartoon for each domain, with the positions of the 57-aa loop and the Velcro closure indicated in FIG. 1B. A Schematic representation of the domains and the corresponding cartoon representation of Bap1 (Left) and RbmC (Right) mutants used in this study is shown in FIG. 1C).

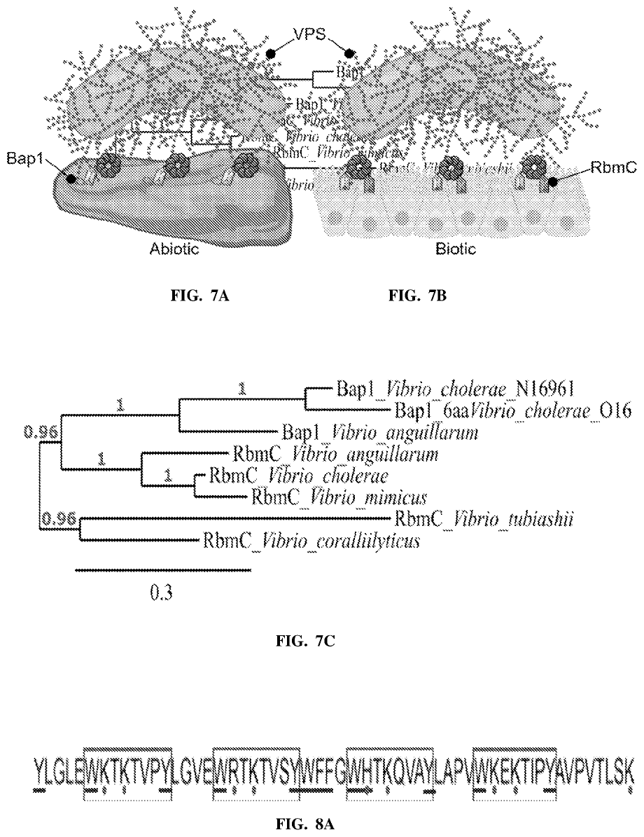

The evolutionary origin of the two matrix proteins were first delved into by performing bioinformatic analyses of Bap1 and RbmC homologues in the Vibrio genus. This is motivated by one interesting genomic feature: While RbmC (VC0930) is located between the two VPS biogenesis clusters along with the other matrix proteins, Bap1 (VC1888) is located outside of the biofilm gene cluster, flanked by genes not related to biofilm structure or regulation. Four other species were found that contain RbmC homologs (V. tubiashii, V. coralliilyticus, V. anguillarum, and V. mimicus) among the 21 Vibrio species analyzed, with two other species showing weak partial hits. Only one species, V. anguillarum, has both a RbmC homolog and a Bap1 homolog. From the protein sequences of the Bap1/RbmC homologues, a phylogenetic tree was generated (FIG. 7C), which shows significant deviation from the species tree (Lin, et al., BMC Genomics 19, 135 (2018)).

This deviation, together with the wider presence of RbmC and the genomic feature mentioned above, indicates that Bap1 likely evolved through a partial gene duplication event from RbmC and was subsequently lost in some Vibrio species but retained in V. cholerae and V. anguillarium. To investigate this, a hypothetical evolutionary intermediate was searched for, a “Bap1” without the 57-aa loop. Although such an intermediate was not found, in one environmental V. cholerae isolate a second copy of Bap1 with a short 6-aa loop was observed. This observation further indicated that Bap1 is potentially a mobile element, and so is the loop. It was conjectured that the ancient gene duplication event and the subsequent sequence evolution could allow Bap1 to acquire new functionality, a process known as “neofunctionalization” (Conant & Wolfe, Nature Reviews Genetics 9, 938-950 (2008)).

To test the difference in the functions of two adhesins and the contribution of their constituent domains, V. cholerae mutants in which one or multiple domains of Bap1 and RbmC are deleted or modified were generated (FIG. 1C), and whenever possible, the corresponding mutant proteins or domain(s) from E. coli attached to a GFPUV label were overexpressed and purified (Crameri, et al., Nature Biotechnology 14, 315-319 (1996)). To modify the protein sequence in the native locus in V. cholerae, the recently-developed MuGENT technique was used (Dalia, et al., Proc. Natl. Acad. Sci. USA 111, 8937-8942 (2014)). In order to focus on the molecular mechanism of biofilm adhesion rather than the confounding effects of gene regulation, a strain background locked in a high cyclic diguanylate level was used that constitutively produces biofilms (i.e. rugose strain) (Beyhan and Yildiz, Mol. Microbiol. 63, 995-1007 (2007)).

Additionally, to study the effects of mutations of individual adhesins in functional assays, a genetic background was used in which the other adhesin is deleted. All constructed strains were complementable with an arabinose-inducible plasmid expressing either wild-type (WT) Bap1 or RbmC.

### Bap1 and RbmC Mutants in the β-Prism Show Adhesion Defects

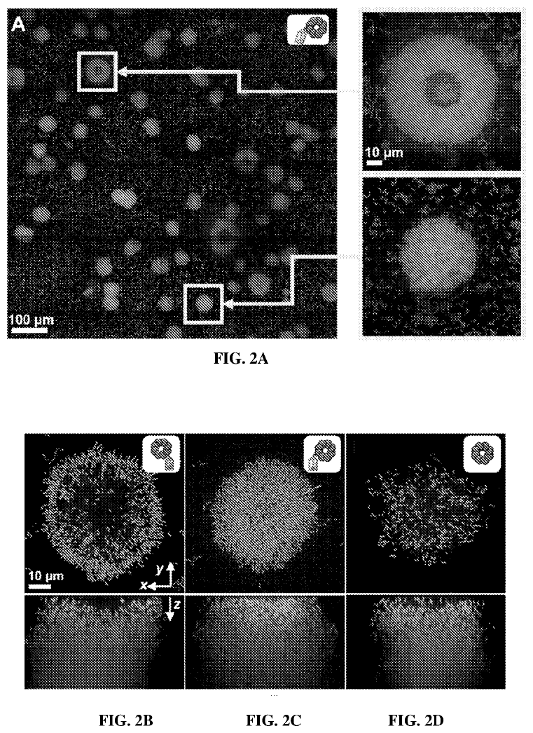

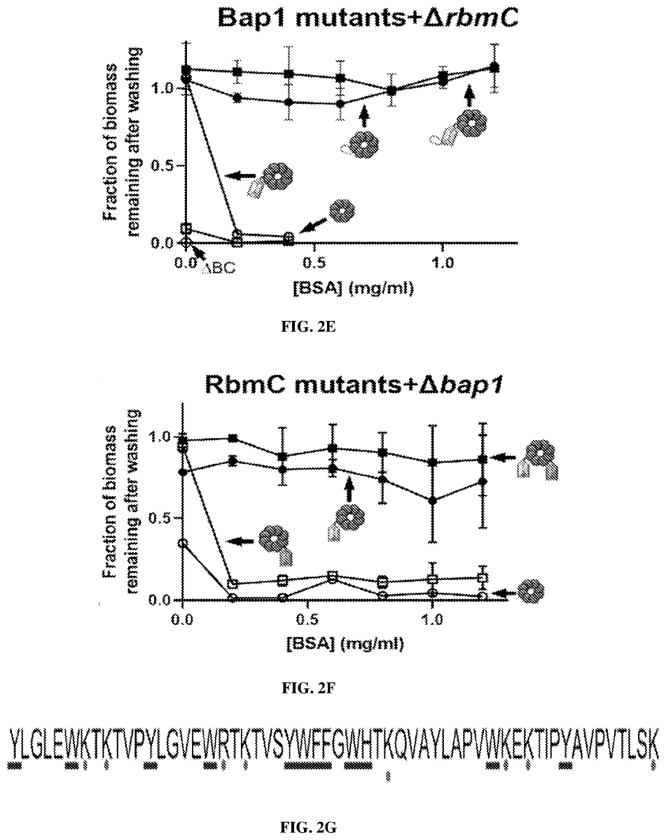

V. cholerae biofilms were first grown on glass substrates and imaged individual cell clusters formed by each mutant using confocal microscopy. Previously, it was shown that in such cell clusters, V. cholerae cells form a densely packed, highly ordered core in which the curved-rod-shaped cells align vertically on the substrate, surrounded by peripheral cells aligning in the radial direction (Hartmann, et al., Nat. Phys. 15, 251-256 (2019), Qin, et al., Science 369, 71-77 (2020), Yan, et al., Proc. Natl. Acad. Sci. USA 113, e5337-5343 (2016)). This organization remains unchanged upon the deletion of either RbmC or Bap1 (Yan, et al., Proc. Natl. Acad. Sci. USA 113, e5337-5343 (2016)). To study the role of each domain in Bap1 and RbmC in promoting biofilm adherence, the biofilm growth was repeated with each of the different constructs shown in FIG. 1C. Interestingly, in the ΔrbmC Δ57-aa mutant, a central void was observed in large cell clusters (diameter >50 μm) not observed in smaller clusters or in the rugose WT (FIG. 2A). It has been previously shown that mechanical stresses during biofilm expansion on solid substrates lead first to cell verticalization and eventually to cell ejection from the substrate (Beroz, et al., Nat. Phys. 14, 954-960 (2007), Nijjer, et al., BioRxiv 2021.05.11.440221 (2021)). The central void observed in large cell clusters in the ΔrbmC Δ57-aa mutant therefore indicates that this strain has diminished surface adhesion.

When the entire β-prismB domain was further deleted, a defective morphology detached from the glass substrate, similar to the ΔrbmC Δbap1 mutant was observed. When investigating RbmC using the same approach, unexpected asymmetry in the role of the two β-prismCs in mediating adhesion was found an: a central void is observed in the Δbap1 Δβ-prismC1 mutant biofilm (FIG. 2B) whereas the Δbap1Δβ-prismC2 mutant displays a normal phenotype indistinguishable from the parental strain (FIG. 2C). Deleting both β-prismCs leads to a nonfunctional RbmC with only a few sporadic cells within the basal layer (FIG. 2D). The microscopy results indicate that the β-prisms play an important role in biofilm adhesion.

To quantitively study the degree to which these mutants affect V. cholerae biofilm adhesion, an assay in which the total biofilm biomass is measured before and after challenging the biofilm clusters with a vigorous washing step was used. The fraction of the biomass remaining on the glass after washing is an indication of adhesion strength. In this assay, biofilms possessing either a WT Bap1 or RbmC will have a value close to 1 and non-adhesive mutant such as ΔrbmC Δbap1 will have a value close to 0. To amplify any differences in adhesion strength between the mutants, Bovine Serum Albumin (BSA) was used to reduce nonspecific binding to the glass substrate (FIG. 2E).

Consistent with its defective biofilm morphology, it was observed that the ΔrbmC Δ57-aa mutant is unable to remain attached to the glass substrate in the presence of BSA (open circles, FIG. 2F). This result confirms that the 57-aa loop is required for robust adhesion of Bapt to glass. On the other hand, a Bap1 construct in which the β-prismB was deleted and the 57-aa directly attached to the β-propeller is fully functional: the resulting biofilms remain attached to surface at all [BSA] tested (closed circles, FIG. 2F). This result is consistent with the idea that the 57-aa loop nested in β-prismB contributes more than the β-prismB per se to biofilm adherence to abiotic surfaces. Results from the deletion of RbmC's β-prisms using this adhesion assay supports the conclusion of a dominant role of β-prismC1 over C2 in abiotic surface adhesion (compare open boxes to filled circles FIG. 2F).

Removal of all β-prisms, either in Bap1 or in RbmC, results in minimal adhesion that is easily abolished by BSA. It has been confirmed that the loss of function in these mutants are unlikely due to changes in the production or secretion level of the mutant protein; for example, overexpressing the Δβ-prismB mutant protein does not rescue its function.

Another common biofilm quantification method is an assay based on crystal violet (CV) binding, which quantifies pellicle formation at the air-liquid interface and adherence to the air-liquid-solid contact (O'Toole, JoVE e2437 (2011)). The CV assay results were generally consistent with the adhesion assay findings, and more importantly, deletion of Bap1 leads to a much more severe adhesion defect than the deletion of RbmC.

The data showed that Bap1 plays a more important role than RbmC in maintaining the biofilm pellicle at the air-liquid interface, echoing prior observation in the literature that on abiotic substrates, Bap1 is the dominant adhesin (Absalon, et al., PLoS Pathog. 7, e1002210 (2011), Hollenbeck, et al., Biophys. J. 107, 2245-2252 (2014), Yan, et al., Adv. Mater. 30, 1804153 (2018)).

### Mutant Proteins Show Different Spatial Distributions in Biofilms

To probe the underlying mechanism of adhesion defects in mutants missing β-prism(s), it was determined mutant constructs exhibited an altered localization within the cell cluster. To do so, the relevant Bap1 and RbmC mutants were tagged with a C-terminal 3×FLAG and used an Anti-FLAG antibody conjugated to Cy3 to label these proteins in situ. Note that in this assay, a functional, WT copy of the alternative adhesin is present to anchor the biofilm to the surface, allowing us to examine the spatial distribution of the mutant proteins in surface-attached biofilms regardless of whether the mutant protein itself is functional. Previous work has shown differential distribution of Bap1 and RbmC (Absalon, et al., PLoS Pathog. 7, e1002210 (2011), Berk, et al., Science 337, 236-239 (2012), Yan, et al., Proc. Natl. Acad. Sci. USA 113, e5337-5343 (2016)). Specifically, while both proteins are distributed throughout the biofilm, Bap1 is particularly concentrated at the biofilm-substrate interface, whereas RbmC is not. These observations were confirmed with a strain expressing WT Bap1 protein. Next, it was observed that the Bap1Δ57-aa mutant completely abolishes the immunosignal at the biofilm-glass interface, while retaining staining at the periphery of the biofilm despite proper protein production and secretion. The same is true for the Bap1 mutant with the β-propeller domain alone. These data are consistent with the reduced adhesion strength of the corresponding biofilms in the adhesion assay: these mutant proteins have a diminished tendency to adsorb to glass surfaces and therefore are defective in adhering the biofilms to the surface. Additionally, the Bap1 construct with the 57-aa directly attached to the β-propeller still exhibits a strong signal at the surface, consistent with its full function in adhesion assays. These observations are consistent with the conclusion that the 57-aa loop in Bap1 is the primary player in promoting V. cholerae biofilm adhesion to abiotic surfaces.

### Positive Charges on the β-Prism Cap Mediates Adhesion to Abiotic Surfaces

Extensive work on adhesive mussel foot proteins (Mfps) highlights the importance of positively charged residues, in conjunction with adjacent aromatic residues, in promoting adhesion to abiotic surfaces (such as negatively-charged mica) in oceanic environments (Gebbie, et al., Nat. Chem. 9, 473-479 (2017), Lee, et al., Annu. Rev. Mater. Res. 41, 99-132 (2011), Li, et al., Nat. Commun. 11, 3895 (2020)). In this regard, the ability of the 57-aa loop to adhere to glass is consistent with its distinct sequence (FIG. 2G): it is rich both in aromatic units such as tyrosine (8.8%) and tryptophan (8.8%) and in positively charged amino acids such as lysine (12.7%). Moreover, in the crystal structure of the Bap1Δ57-aa, the cap of the β-prismB domain (facing away from the β-propeller) shares similar features (Kaus, et al., J. Biol. Chem. 294, 14499-14511 (2019)). Therefore, the β-prismB cap together with the 57-aa loop may form a continuous, positively charged surface enabling Bap1 to function as the predominant adhesin interacting with abiotic surfaces.

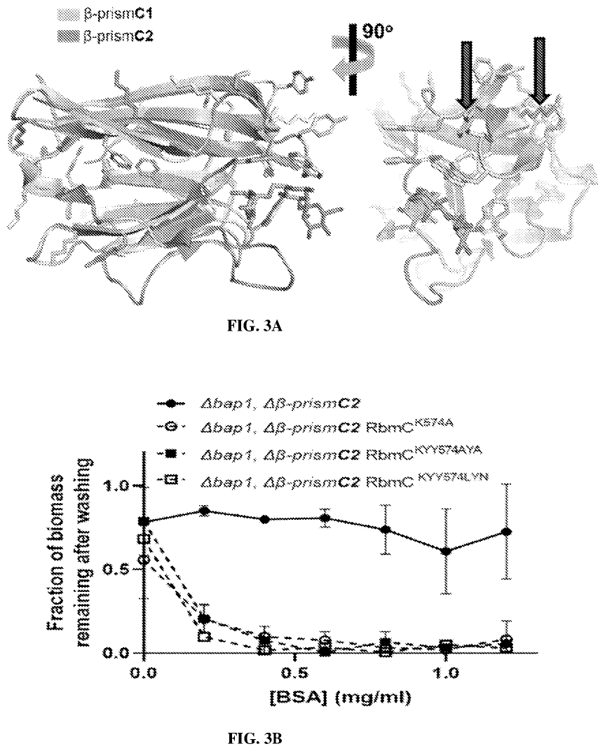

Many prior studies have claimed redundancy regarding Bap1 and RbmC's function. Indeed, it was observed that RbmC possesses residual adhesion to glass surfaces under the experimental conditions, mainly due to β-prismC1, which does not have a loop. Following the analogy with Mfps, experiments were designed to test if the same positive-charge/aromatic residue combination may explain this residual adhesion and also why β-prismC1 is superior to C2 in the adhesion assay. To visualize this, electrostatic surface maps of the three β-prism domains were generated. Indeed, the surface charge follows the order β-prismB>C1>C2. Moreover, it was observed that an outward facing lysine (K574) is present on the cap of β-prismC1 but not C2. Closer analysis of the nearby surface region reveals a sequence of KYY in β-prismC1, compared to LYN in β-prismC2 in the same location (FIG. 3A). Next, site-specific mutations were introduced in D-prismC1 converting KYY to LYN and the surface adhesion ability of the resulting mutant was examined. To show that the results are not due to a detrimental effect of L and/or N, a mutant in which both K and Y are mutated to alanine (AYA) was generated. Results show that removing the lysine and tyrosine in this position significantly reduced the adhesive properties of β-prismC1 to the glass surface (FIG. 3B). In fact, a single point mutation from K to A (AYY) is sufficient to reproduce the same loss of adhesion. These results demonstrate that small differences in the cap sequence of β-prisms, i.e., at the interface between the biofilm and the external environment, can dramatically change the ability of the adhesins to adsorb on surfaces. This sensitivity could be because, in biofilms, small differences in the binding affinity at the molecular level can be amplified by an intrinsic, macroscopic “avidity” effects (Erlendsson & Teilum, Front. Mol. Biosci. 7, 2020.615565 (2021)) due to the numerous molecular contacts at the biofilm-substrate interface.

### RbmC, but not Bap1, Binds to N-Glycans on Host Cell Surfaces

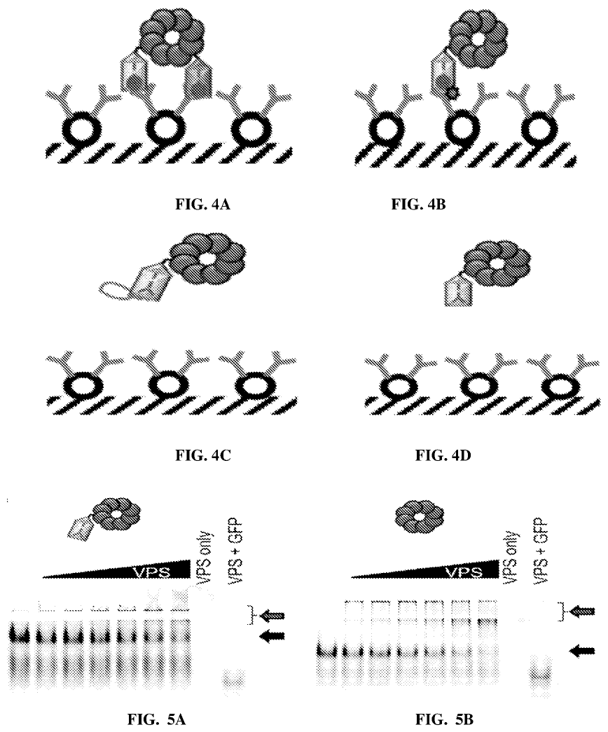

The structural and sequence differences between β-prismB and Cs lead to the design of experiments to test if RbmC and Bap1 behave differentially when interfacing with biotic surfaces that V. cholerae might encounter during colonization and infection. Previously, a glycan array analysis was performed, which indicated specific binding of β-prismCs to complex N-glycans prevalent on the surface proteins of host cells (De, et al., PLOS Pathog. 14, e1006841 (2018)). Subsequently, it was shown that β-prismCs can bind to the core branch-region of N-glycans with nanomolar affinity, and a model based on crystal structures of glycan fragments bound to β-prismC2 was generated. All these data strongly support the potential of RbmC to bind tightly to host cell surfaces. Indeed, using Caco-2 cells as a model for host cells that V. cholerae may encounter during colonization (Cont, et al., ELife 9, e56533 (2020)), it was shown that purified, GFP-tagged β-prismC1 and C2 both show specific binding to these cells (FIG. 4A). This binding is a direct consequence of the N-glycan binding pocket identified earlier through crystallography (De, et al., PLOS Pathog. 14, e1006841 (2018)), because mutation of a key aspartic residue (D539A) in this binding pocket can abolish the β-prismC1's binding to Caco-2 cells. Interestingly, binding of β-prismB or any Bap1 construct to Caco-2 cells was note observed. Consistent with the absence of Caco-2 cell binding, a glycan array analysis with Bapt failed to identify any mammalian glycan targets (Kaus, et al., J. Biol. Chem. 294, 14499-14511 (2019)). In addition to the three β-prisms in Bap1 and RbmC, the V. cholerae genome contains another β-prism in V. cholerae cytotoxin (VCC), and similar to RbmC, VCC also uses this β-prism domain to bind to complex N-glycans on host cell surfaces (Levan & Olson, J. Mol. Biol. 425, 944-957 (2013)).

To test the glycan-binding ability of different mutant V. cholerae biofilms, a convenient in vitro assay was developed: biofilms are grown on glass substrates coated with asialofetuin, an extensively glycosylated protein (Windwarder & Altmann, Journal of Proteomics 108, 258-268 (2014)), and in situ immunostaining is performed as before. Interestingly, the surface staining pattern of Bap1 and RbmC is “reversed” in the presence of asialofetuin compared to that on bare glass (FIGS. 4B, 4C): RbmC shows a strong signal concentrated at the core of the asialofetuin-coated glass substrate, whereas Bap1 only shows a peripherical staining pattern around cell clusters. The evidence described above indicates that Bap1, during its evolution towards a dedicated adhesin for abiotic surfaces, has lost the ability to bind host cell surfaces. Using β-prismC1 and C2 as a template, mutations were attempted to be introduced into the β-prism B domain to restore N-glycan binding to determine if they could restore N-glycan binding ability, but this was not successful indicating a significant departure from glycan-binding activity for the Bap1 β-prism domain.

An interesting feature of 1-prismC1 is that the glycan binding pocket seems to be orthogonal to the surface responsible for abiotic surface adhesion, which resides on the other side of the cap (FIG. 4A). To illustrate this point, an asialofetuin binding assay was performed with the rbmCK574A mutant that exhibits defective glass-surface adhesion. Indeed, this mutant still retains the ability to coat asialofetuin-coated surfaces in a manner indiscernible from the parental strain (FIG. 4C). In contrast, the β-prismC1 mutant missing the key aspartic acid residue in the glycan binding pocket (D539A) abolishes asialofetuin binding but retains a WT-level of adhesion to the abiotic glass surface (FIG. 4D). This confirms that the glycan-binding pocket in β-prismC1 functions independently from the glass-adhering surface, rendering β-prismC1 a bi-functional adhesive domain.

A conserved β-Propeller Domain Anchors Bap1 and RbmC to VPS

Next, the question of what makes Bap1 and RbmC “biofilm-specific” as opposed to classical adhesins that function at a cellular level was asked. The key to this answer lies in the connection between the adhesins and the main structural component of the V. cholerae biofilm, VPS. It was further hypothesized that the conserved β-propeller domain anchors RbmC/Bap1 to VPS Because VPS is only synthesized by V. cholerae cells in a biofilm but not planktonic state (Yildiz, et al., PLoS ONE 9, e86751 (2014)), this connection renders RbmC/Bap1 biofilm-specific. According to the crystal structure (FIG. 1B), Bap1's β-propeller is a ring-like domain with eight blade repeats, each of which contains a four-stranded β-sheet in a twisted configuration. The three strands of the last blade are circularly permutated with the N-terminus of the protein, which contains a single β-sheet; hydrogen bonding between the two generates a molecular “Velcro closure” that stabilizes the circular shape of the β-propeller (Chaudhuri, et al., Proteins: Structure, Function, and Bioinformatics 71, 795-803 (2008)). Although there is no crystal structure of the RbmC β-propeller, due to the high degree of sequence conservation between the two β-propellers it is believed that they will have a similar structure.

To investigate the functional role of the β-propeller several complementary methods were used. First, was shown that mutants with an intact 1-propeller but no β-prism(s), both in Bap1 and RbmC, are defective in the adhesion assay (FIGS. 2A-2F) yet show staining on the periphery of the biofilm with immunolabeling. Second, E. coli-purified domain(s), tagged with GFP, were added to mature V. cholerae biofilms (FIG. 5A). Consistent with the hypothetical VPS-β-propeller interaction, it was observed that any construct containing an intact β-propeller domain shows a positive staining signal. Moreover, the interaction between the 1-propeller domain and biofilm cells is VPS-dependent, as ΔvpsL cells lacking the ability to produce VPS do not bind to the GFP-labelled proteins. These results confirm that the 1-propeller binds to VPS and not directly to V. cholerae cells, and this binding explains why Bap1 and RbmC specifically function in the context of the biofilm but not in the initial attachment of planktonic cells to a surface.

The binding between VPS and the 1-propeller domain is direct as opposed to through other intermediate factors. To determine this end, VPS were first purified according to a modified procedure (Yildiz, et al., PLoS ONE 9, e86751 (2014)), starting from a ΔrbmAΔbap1ΔrbmC mutant strain with overproduction of VPS but no matrix proteins to avoid confounding factors. An electrophoretic mobility shift assay (EMSA) was then performed under non-denaturing conditions with several GFP-tagged protein constructs and purified VPS at increasing concentrations (FIG. 5B). For constructs that contain the β-propeller, a decrease in the intensity of the unbound protein bands and simultaneously, the emergence of slower moving, high molecular weight bands or bands that do not enter the gel, were observed indicating the formation of large aggregates (Nero, et al., Nucleic Acids Research 46, 6099-6111 (2018)). In contrast, the GFP-tagged β-prismB and the GFP-only negative controls do not show a decrease in signal intensity nor the appearance of large aggregates. The same observation was confirmed with β-propeller from RbmC. These results provide evidence of the direct binding between VPS and the β-propeller in solution.

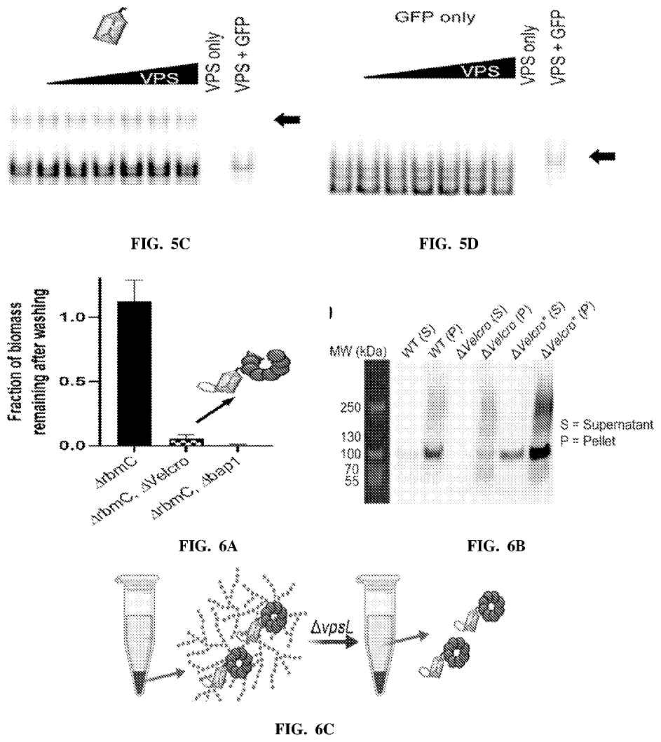

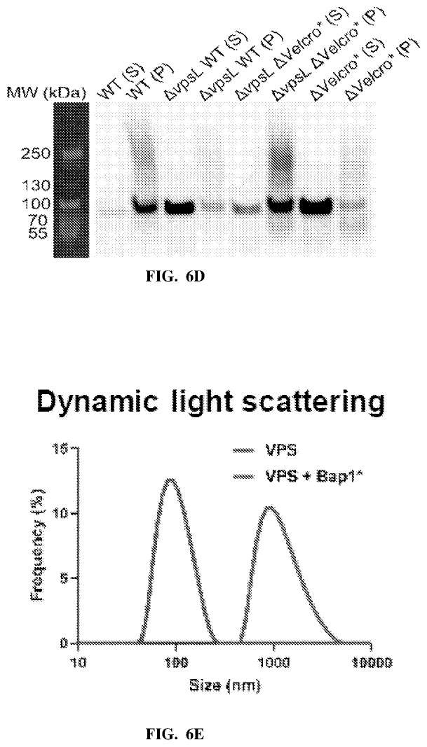

To further test this interaction in V. cholerae biofilms, it was attempted to generate Bap1/RbmC mutants in V. cholerae without the β-propeller domain. These constructs are not properly secreted, presumably because the Type-IL secretion machinery recognizes certain sequences on the Q-propeller required for secretion (Johnson, et al., J. Bacteriol. 196, 4245-4252 (2014), Reichow, et al., Nat. Struc. Mol. Biol. 17, 1226-1232 (2010)). As an alternative strategy, a Bap1 construct was generated in which the Velcro sequence at the N-terminus of blade 1 is removed (Bap1ΔVelcro). Previous work with other β-propellers have shown that the removal of the Velcro closure that locks the ends of the complex in a circular conformation leads to a nonfunctional but stable domain (Bonsor, et al., The EMBO Journal 28, 2846-2857 (2009), Mylemans, et al., Protein Science 29, 2375-2386 (2020)). It was reasoned that such manipulation will result in a secretable Bapt protein with a nonfunctional β-propeller domain with intact surface adsorption but an interrupted VPS interaction. Indeed, the Bap1ΔVelcro construct is successfully secreted, but is defective in adhering V. cholerae biofilms to the surface in the ΔrbmC background (FIG. 6A).

To uncover the root of the dysfunction of Bap1ΔVelcro, the corresponding 3×FLAG-tagged version was made and immunostaining performed as before. Unlike the nonfunctional Bap1Δβ-prismB mutants (FIG. 3B), the Bap1ΔVelcro construct shows a staining pattern at the biofilm-glass interface indistinguishable from that of WT Bap1, indicating that the mutant protein itself can adsorb properly to the glass substrate due to its intact β-prismB with the 57-aa loop. Interestingly, in the bulk of the biofilm, the Bap1ΔVelcro construct shows puncta-like structures on the order of 200-500 nanometers throughout the biofilm, in contrast to the envelope pattern formed by WT Bap1. The altered spatial distribution of Bap1ΔVelcro in the biofilm bulk indicates that the VPS-β-propeller binding is perturbed. Degradation of Bap1ΔVelcro could lead to the perturbed spatial distribution (FIG. 6B). To rule out the effects of degradation, hapA and ivaP, the two major extracellular proteases in V. cholerae (Hatzios, et al., Nat. Chem. Biol. 12, 268-274 (2016), Smith, et al., Proc. Natl. Acad. Sci. USA 112, 10491-10496 (2015)) were deleted to resolve the degradation issues (FIG. 6B). Still, the same staining pattern was observed in the protease-deficient mutant, indicating that the disrupted function of Bap1ΔVelcro is not due to degradation.

Although VPS-β-propeller binding is perturbed in the Bap1ΔVelcro mutant, it is not completely abolished. The puncta-like structures are not associated with individual cells but are rather retained in the biofilm matrix. Moreover, free-floating puncta was not observed outside of the biofilm, nor in non-tagged biofilms in a co-culture. These observations indicate that there are still some residual interactions between VPS and Bap1ΔVelcro. Indeed, Western blots show that Bap1ΔVelcro coprecipitates with the cell-associated VPS, while in the VPS-background (ΔvpsL), Bap1ΔVelcro is found instead mainly in the supernatant (FIG. 6C). The aggregation of the Bap1ΔVelcro construct into puncta may be related to the tendency of the 57-aa loop to drive aggregation—indeed, even on denatured Western blots, constructs with the 57-aa sequence generally display extended, slower moving bands that are absent in constructs without the 57-aa loop (FIG. 6D).

### A Model for Overlapping but Distinct Function of Bap1 and RbmC

With all the evidence above, a model was developed in which RbmC and Bap1 promote V. cholerae biofilm adhesion to divergent types of external surfaces with their differing β-prism domains while sharing conserved β-propeller domains that bind VPS, thereby acting as a unique bacterial “double-sided tape” (FIGS. 7A-7B). Specifically, it is believed that Bap1 specializes in sticking to abiotic surfaces via the mussel-like chemistry while RbmC mainly mediates binding to host cell surfaces through the glycan-binding pockets. Consequently, V. cholerae biofilms can adhere to a wide range of foreign surfaces with different chemical properties in the aquatic environment and during host colonization/infection.

### Summary

A combination of microscopy, bacterial genetics, and biochemical approaches were applied to delve into the fundamentals of biofilm adhesion in Vibrio cholerae. The data indicate that biofilm adhesins in V. cholerae adopt a modular approach that, through evolution, acquired specialized functionalities to attach to diverse surfaces while maintaining their affinity for the native exopolysaccharide. The two adhesins Bap1 and RbmC rely on a conserved β-propeller domain to anchor them to the biofilm via VPS binding, making them biofilm-specific adhesins in contrast to classic adhesins, which attach directly to bacterial cell surfaces. On the other hand, the two adhesins differ in the adhesive properties of their β-prism domains that interface with the external environment. While RbmC's β-prisms show tight binding to N-glycans and consequently host cell surfaces, Bap1's β-prism has evolved to specialize in adherence to abiotic surfaces while losing its N-glycan binding ability. Notably, during evolution, Bap1 acquired a 57-aa loop inside its β-prism domain, which shares some sequence similarity with mussel adhesion proteins. This 57-aa loop is important for robust adhesion to abiotic surfaces. Through the division of labor between these two adhesins previously thought to be functionally redundant, arises a strategy for how V. cholerae biofilms recognize host surfaces specifically during infection while retaining generic adhesion to abiotic surfaces encountered in their natural niches. The existence of these biofilm-specific adhesins in several Vibrio species other than V. cholerae implies that this strategy may apply to other Vibrios. The conceptual, double-sided-tape-like design may be generalizable to other biofilm-forming species, although the specific biochemistry of surface adhesion and protein-polysaccharide binding may vary from species to species.

It is interesting to compare these findings with those of RbmA, the other major matrix protein in V. cholerae biofilms (Fong, et al., J. Bacteriol. 188, 1049-1059 (2016), Teschler, et al., Nat. Rev. Microbiol. 13, 255-268 (2015)). RbmA's primary function is to adhere biofilm cells to each other and to VPS, thereby controlling the compactness and integrity of V. cholerae biofilms (Absalon, et al., PLoS Pathog. 7, e1002210 (2011), Berk, et al., Science 337, 236-239 (2012), Yan, et al., Proc. Natl. Acad. Sci. USA 113, e5337-5343 (2016)). Structural and genetic work further demonstrated that RbmA binds VPS directly using its FnIII domains (Fong, et al., ELife 6, e1002210 (2017)). RbmA shares no sequence or structural homology with RbmC/Bap1. It has no additional domains to interface with foreign surfaces, consistent with its major role in adhering cells to each other and to VPS. It is interesting to note that V. cholerae has evolved two sets of proteins with structurally different sugar-binding domains that both recognize the same carbohydrate polymer: VPS.

The presence of RbmC and Bapt homologs in other Vibrio species indicate that the adhesion biochemistry revealed here could be conserved in those species. Among them, V. coralliilyticus is a coral pathogen that causes coral bleaching (de O Santos, et al., The ISME Journal 5, 1471-1483 (2011)), V. anguillarum is a fish pathogen (Frans, et al., Journal of Fish Diseases 34, 643-661 (2011)), and V. tubiashii infects mollusks (Hasegawa, et al., Appl Environ Microbiol 74, 4101-4110 (2008)).

While biofilm formation has been reported in these species, the biochemical component of these biofilms is unknown, as well as the role of biofilm formation in their infection pathology. The existence of biofilm-specific adhesin homologs in these species indicate that they may produce exopolysaccharide similar to VPS. If true, an intriguing question thus arises as to whether VPS synthesized by one Vibrio species can be recognized by the RbmC or Bap1 secreted by another species. Such crosstalk has been observed in autoinducer recognition in quorum sensing, the process in which bacteria communicate with each other through small molecules (Papenfort & Bassler, Nat. Rev. Microbiol. 14, 576-588 (2016)).

Going beyond the Vibrio genus, matrix proteins are commonly found in many other biofilm-forming species, but in general structural and biochemical information is lacking about how they function at the molecular level, in particular during adhesion. For example, CdrA has been proposed to increase the elasticity of P. aeruginosa biofilms by crosslinking the main exopolysaccharide PsI (Kovach, et al., NPJ Biofilms Microbiomes 3, 1 (2017), Melia, et al., Proc Natl Acad Sci USA 118, e2109940118 (2021), Reichhardt, et al., MBio 9, e01376-18 (2018)). However, it is believed a role in surface anchoring has not been previously proposed. Instead, adhesion to surfaces has been shown to be achieved by Psl and Pel, the two major exopolysaccharides in P. aeruginosa (Cooley, et al., Soft Matter 9, 3871-3876 (2013)), in conjunction with pili (Laventie, et al., Cell Host Microbe 25, 140-152.e6 (2019), Luo, et al., MBio 6, e02456-14 (2015), Siryaporn, et al., Proc Natl Acad Sci USA 201415712 (2014)). It is tantalizing to contemplate how and why P. aeruginosa uses exopolysaccharides rather than proteins to achieve adhesion, or if it possesses additional biofilm-specific adhesins yet to be discovered. Another notable example is BslA in Bacillus subtilis biofilms (Hobley, et al., Proc. Natl. Acad. Sci. USA 110, 13600-13605 (2013), Hobley, et al., FEMS Microbiol. Rev. 39, 649-669 (2015)). BslA has been shown to be a hydrophobin that coats and renders B. subtilis biofilms hydrophobic. BslA self-assembles in vitro at the air-liquid interface into an elastic film, similar to small-molecule surfactants. V. cholerae biofilms are also hydrophobic, and previous results have shown that this hydrophobicity arises from Bap1 (Hollenbeck, et al., Biophys. J. 107, 2245-2252 (2014), Yan, et al., Adv. Mater. 30, 1804153 (2018)). However, in contrast to BslA, the function of Bap1 relies on its binding to VPS: colonies from mutants unable to make VPS, such as ΔvpsL, are hydrophilic. Therefore, the hydrophobicity of V. cholerae biofilms is a byproduct of Bap1's main function of adhesion to abiotic surfaces, which relies on the existence of extensive aromatic residues on the cap of β-prismB and in the 57-aa loop. Indeed, water contact angle measurements show that these features contribute additively to the hydrophobicity of V. cholerae biofilms. Consistent with this argument, RbmC, missing the loop and extensive aromatic residues on either of its β-prisms, does not contribute to the hydrophobicity of the biofilm.

Many bacteria, in particular pathogens, express adhesins on their surfaces and therefore adhere to host cells. Many such adhesins use a modular approach. For example, Staphylococcus aureus possesses >20 cell-wall-anchored adhesins that bind eukaryotic host factors like fibronectin (Foster, et al., Nat. Rev. Microbiol. 12, 49-62 (2014), Mazmanian, et al., Science 285, 760-763 (1999)). In this manner, they are designed to function at the level of the individual bacterium. Another example in V. cholerae is GbpA (Kim, et al., Nature 438, 863-866 (2005)). GbpA contains four separate domains, two of which bind to chitin and the remaining two which are required for interacting with the bacterial cell surface (Wong, et al., PLOS Pathog. 8, e1002373 (2012)). Therefore, while the modular approach is conceptually similar, the recognition of the bacterial cell surface by classical adhesins is tied to single-cell behavior.

The results address many questions regarding the molecular mechanism underlying V. cholerae biofilm adhesion. For example, the β-propeller domain directly binds to VPS, which might explain the close juxtaposition of RbmC and VPS signals observed in super-resolution microscopy (Berk, et al., Science 337, 236-239 (2012)). It is likely that VPS-β-propeller binding is multivalent: previous data from mechanical measurements indicate crosslinking of VPS by RbmC/Bap1 (Yan, et al., Adv. Mater. 30, 1804153 (2018)), which requires one RbmC/Bap1 molecule to bind to two or more VPS monomers. Indeed, the high-molecular weight bands in the EMSA may likely arise from the crosslinking of VPS by the β-propeller.

The phylogenetic analysis indicates that the 57-aa loop was acquired after an ancient gene duplication event, however, the origin of the 57-aa is difficult to trace. Since many Vibrio species including V. cholerae are naturally competent (Meibom, et al., Science 310, 1824-1827 (2005)), the long loop might be derived from environmental DNA sources rather than a gradual evolutionary process. Biochemistry wise, the prevalence of lysine and aromatic residues in the 57-aa sequence warrant future studies on different variations of the loop.

There are two additional domains with high sequence identity at the N-terminus of RbmC. They are homologous to the C-terminal domain of StcE, the secreted mucinase of enterohemorrhagic E. coli (Yu, et al., Structure 20, 707-717 (2012)). Recent work by Nason et al. shows that this domain (X409 in SctE) binds to but is not responsible for mucin degradation (Nason, et al., Nature Communications 12, 4070 (2021)). This comparison indicates an interesting scenario that RbmC, in addition to anchoring V. cholerae biofilms to N-glycosylated host cell surfaces, may also assist in adhering to the intestinal mucus layer. If true, this scenario will further establish RbmC as the host-specific adhesin whereas Bap1, lacking a functional N-glycan-binding (3-prism and the additional biotic-motif-binding domains, has evolved to be an abiotic-surface-specific adhesin.

### Example 2: Wet Adhesives Derived from Vibrio cholerae Biofilm Adhesins for Industrial and Biomedical Applications

### Methods

In Vc cells, the 57-aa sequence nested in the adhesion protein Bap1 helps Vc biofilms adhere to abiotic surfaces. Data presented here show the adhesive properties of the 57-aa based on in vitro characterization using a chemically synthesized peptide. The amino acid sequence of the 57-aa loop nested in Vibrio cholerae adhesin responsible for biofilm adhesion to various abiotic surfaces is depicted in FIG. 8A-8B, with positive residues highlighted by dots and aromatic residues underlined, with the repeating motif highlighted by boxes. A sequence logo showing the most conserved amino acids in the repeating motif is depicted in FIG. 8C.

### Media

Most of the measurements were performed in M9 minimal media (Sigma Aldrich) supplemented with 2 mM MgSO4 (JT Baker) and 100 μM CaCl2) (JT Baker) (henceforth referred to as M9 media). This media was extensively used to grow V. cholerae biofilm; and was therefore used for adhesion measurement for consistency.

### NaOH Treatment of Glass Substrates

100 μL of 10 M NaOH aqueous solution was added to the wells of the 96-well plate and incubated at room temperature for 10 minutes, after which the wells were washed with DI water until the pH was neutral. The treated wells were kept hydrated with DI water prior to use. This treatment is necessary to minimize spontaneous adsorption of the peptide in the fluorescence-based assay described below.

### Microbeads Adsorption Assay

Chemically synthesized, FITC-labeled peptides (Atlantic Peptides or Lifetein) or FITC (Fisher Scientific F0026100MG) were dissolved in DMSO at a 150 μM stock concentration and stored at 4° C. 5 am microspheres/beads with different surfaces: silica (Polysciences 25348), carboxylate-modified latex (Thermo Fisher C37255), and sulfate-modified latex (Thermo Fisher S37227), were also diluted to 1% (weight percent) stock solutions in MilliQ water. Immediately prior the adsorption assay, M9 media containing 0.2 mg/ml BSA and 0.01% wt % of 5 μm silica microspheres (Polysciences 25348) was made, followed by preparing dilutions of the labeled peptide in DMSO in PCR tubes to a series of concentrations ranging from 0.005 μM to 3 μM. 1 μL of peptide at each concentration was quickly added to 99 μL of the M9 media with 0.2 mg/ml BSA (Sigma-Aldrich A9418) in another PCR tube, then briefly vortexed. (NOTE: the peptide/DMSO dilutions were prepared immediately prior to mixing with the beads to minimize effects of peptide adsorption to the tube over time.) Afterwards, the peptide-bead mixtures were gently shaken for 30 mins at room temperature, shielded from light to avoid photobleaching. This was followed by bath sonication for 20 min with ice before transferring to a NaOH-treated 96-well plate with a glass bottom (MatTek P96G-1.5-5-F) and allowing to settle at room temperature for 5 min before imaging. The prepared samples were imaged with a spinning disk confocal microscope (Nikon Ti2-E connected to Yokogawa WI) using a 60× oil objective (numerical aperture=1.40) and a 488 nm laser excitation or bright field. For each sample, at least three locations were imaged and captured with a sCMOS camera (Photometrics Prime BSI). Each field of view contained roughly 100-150 beads.

The 57-aa peptide (SEQ ID NO:1) and its variants were tested for adsorption assays. Below is a list of these variant sequences:

(SEQ ID NO: 37)
YLGLEWATATVPYLGVEWATATVSYWFFGWATAQVAYLAPVWAEATIPY
AVPVTLSK,
(SEQ ID NO: 38)
YLGLELKTKTVPLLGVELRIKTVSLWFFGLHTKQVALLAPVLKEKTIPL
AVPVTLSK,
(SEQ ID NO: 39)
YLGLEWKAKAVPYLGVEWRAKAVSYWFFGWHAKQVAYLAPVWKEKAIPY
AVPVTLSK,
(SEQ ID NO: 40)
YLGLEAKTKTVPALGVEARTKTVSAWFFGAHTKQVAALAPVAKEKTIPA
AVPVTLSK,
(SEQ ID NO: 41)
YLGLEWKTKTVPYWRTKTVSYWHTKQVAYWKEKTIPYAVPVTLSK,
(SEQ ID NO: 42)
YLGLEWKTKTVPYLGVEWRTKTVSYLGPEWHTKQVAYLAPVWKEKTIPY
AVPVTLSK,
(SEQ ID NO: 43)
WKTKTVPY,
(SEQ ID NO: 44)
LGLEWKTKTVPYLGVEWRTKTVSY,
(SEQ ID NO: 45)
VSYWFFGWHTK.

### Quantification of Microbeads Adsorption Assay

Background noise in the 488 nm channel was measured by taking images of M9 medium and quantifying with built-in functions of Nikon Element software. Signal intensity per unit area in the solution or in the adsorbed layer of the beads was measured by a customized code written in MATLAB program and the difference was calculated as a measurement of excessive molecular concentration on bead surface.

### Silane Chemistry Modification

5 μm dry silica beads (polysciences 25348) were placed in an 1.5 mL Eppendorf tube with lid open and incubated with the vapor of 1 mL (3-aminopropyl) triethoxysilane or triethoxy(propyl)silane at room temperature for 2 days.

### Zeta Potential Measurement

The pH of a 10 mM NaCl solution was adjusted to be 3 or 7 with a NaOH solution or a HCl solution. Silica beads and latex beads were resuspended in the 10 mM NaCl solution (0.01% silica and 0.04% latex) and added to the cell for zeta potential measurements using a dynamic light scattering equipment (Horiba) (FIG. 6E).

### Atomic Force Microscopy

AFM measurements were performed using an Asylum Cypher ES atomic force microscope (Asylum Research). A 5 μm silica bead (polysciences 25348) was attached to a tipless probe (Nanoworld, PNP-TR-TL-AU) with UV curable glue. The spring constant of the probes was calibrated using the thermal method. The AFM was operated in contact force mode. 1.5 μM 57-aa M9 solution was placed on a mica substrate, and the cantilever with glued bead was also submerged in the 1.5 μM 57-aa M9 solution. The probe was operated to approach the mica substrate, also precoated with the 57-aa peptide, at a velocity of 500 nm/s and a dwell of 5 seconds before retraction, and the force was recorded and plotted over distance to generate a force curve.

### FACS-Based Adsorption Assay

FITC-labeled 57-aa or FITC alone was diluted with M9 medium with 0.01% beads to 1.5 M. The mixture was vortexed and incubated at room temperature with shaking for 30 minutes before sending to FACS (BD FACS LSR Fortessa X20). 10,000 events were recorded, in which 99% were selected and plotted to give a histogram.

### Lipid-Coated Microbead Adhesion Assay

TABLE 1
SUV compositions

| SUV lipids | Lipid-molar ratio (%) * |
|---|---|
| DOPC/DOPS/PIP2 | 75:20:5 |
| DOPC/DOPS | 75:25 |
| DOPC | 100 |
| DOPC/Cholesterol | 50:50 |
| DOPC/Cholesterol/Sphingomyelin | 33.4:33.3:33.3 |

* DOPC mol % has been rounded up by 0.1 for all compositions. This remaining 0.1% corresponds to Rhodamine-PE, used to label all SUVS.

Silica microbeads were coated with lipid layers according to published protocols with modification (Bridges, et al., J. Cell Biol. 213, 23-32 (2016)). Lipids DOPC (Avanti Polar Lipids 850375C), DOPS (Avanti Polar Lipids 840035C), PIP2 (Avanti Polar Lipids 850155P), Brain Sphingomyelin (Avanti Polar Lipids 860062C), Cholesterol (Sigma-Aldrich C3045) and L-α-phosphatidylethanolamine-N-(lissamine rhodamine B sulfonyl) (abbreviated as RhPE, Avanti Polar Lipids 810146), were purchased to prepare small unilamellar vesicles (SUVs) with the compositions shown in Table 1. Briefly, the lipids were mixed in chloroform inside a glass vial that was cleaned with chloroform. The vial was placed underneath a light stream of nitrogen for 15 mins to evaporate excess solvent, followed by at least 2 h in a vacuum desiccator. Afterwards, the lipids were rehydrated for 30 min at 37° C. at a final lipid concentration of 5 mM in buffer (20 mM Tris, pH 8.0, 300 mM KCl, and 1 mM MgCl₂) with vortexing and rough agitation every 5 min, followed by probe sonication to clarity (4 min, with intermittent breaks) to form small unilamellar vesicles (SUVs). The SUVs were adsorbed onto 5 μm silica microspheres by mixing 50 nmol lipids with 440 mm² of silica microspheres surface area in a final volume of 80 μL and 1 h rotary shaking at room temperature. Excess SUVs were removed by pelleting coated beads for 30 s at 862×g (3030 rpm), followed by washing 4 times with excess buffer (100 mM KCl and 50 mM Tris, pH 8.0). Instead of M9 buffer, the lipid-coated beads were diluted to 0.01% in a 2^{nd} sample buffer (100 mM KCl, 50 mM Tris, pH 8.0, 0.1% methylcellulose (Sigma-Aldrich M7027), 0.1% BSA). Then, a series of dilutions of the labeled peptide were prepared in DMSO in PCR tubes. For each dilution, 1 μL was quickly added to 99 μL of the M9 media with silica microspheres in another PCR tube, then briefly vortexed. (NOTE: the peptide/DMSO dilutions were prepared immediately prior to mixing with the beads to minimize effects of peptide adsorption to the tube over time.) After adding the FITC-labeled peptide to the bead solutions, samples were shielded from light in a small box to avoid photobleaching, and gently shaken for at least 1 h at room temperature. Afterwards, prepared samples were imaged with a spinning disk confocal microscope (Nikon Ti2-E connected to Yokogawa WI) using a 60× water objective and a 488 nm laser excitation or a 561 nm laser excitation. For each sample, at least three locations were imaged and captured with a sCMOS camera (Photometrics Prime BSI). Each field of view contained roughly 100-150 beads

Assays with Mice

All animal work was approved by Yale University's Institutional Animal Care and Use Committee. CD1 (Charles River) mice of both sexes, aged 4-12 weeks were used in this study. CDl IGS mice were purchased from Charles River Laboratories, Strain 022 and were bred for up to two generations within the Yale Animal Resource Center. Mice were maintained in ventilated Techniplast limit racks with ambient temperature of 22° C. and 50%±10% humidity in a barrier facility with 12 hour light/dark cycles. They were given ad libitum access to food and water.

### Enteroid Monolayer Generation and Culture

96-well Black/clear plates (Corning 353219) were coated with 30 μL growth factor reduced Matrigel (Corning 356231) diluted 1:5 in Basal organoid medium. Plates were incubated at 37° C. for at least 1 hour to allow Matrigel to polymerize. Basal organoid medium was comprised of: advanced DMEM/F12 (Thermo Fisher 12634010) supplemented with 1×N-2 supplement (Thermo Fisher 17502-048), 1×B-27 supplement (Thermo Fisher 17504044), 10 mM HEPES (AmericanBio AB6021-00100), 1× Glutamax (Thermo Fisher 35050061), 1 mM N-acetyl-cysteine (Sigma Aldrich A9165), and 1× Penicillin/Streptomycin (Thermo Fisher 15140-122).

Enteroid monolayers were generated as described previously (Thome, et al., Dev. Cell 44, 624-633.e4 (2018) with modifications. Briefly, ˜4 cm of jejunum was removed from 4-12 week old mice, flushed with ice-cold PBS and cut open longitudinally to expose the epithelium. The tissue was scraped with a 22×22 mm coverslip to remove villi and placed in PBS+3 mM EDTA at 4° C. with rotation for 30 minutes. The tissue was then manually shaken with forceps in a 6 cm petri dish to release villi. The PBS was replaced to deplete villar fractions. This process was repeated until the PBS contained mostly crypts by visual inspection. The solution containing crypts was strained through a 70 μm filter (Fisher) and centrifuged at 300×g for 3 minutes to pellet crypts. Crypts were resuspended in 2D attachment media, which consisted of basal organoid media (above) supplemented with 50 ng/mL EGF (Thermo Fisher PMG8041), 100 nM LDN-193189 (Cayman, 11802), 1 μg/mL R-spondin 1 (R&D Systems, 3474-RS-050), 10 μM CHIR99021 (Cayman, 13122), and 10 μM Y27632 (Tocris, 1254). 100 μL of resuspended crypts were added to each Matrigel-coated well of a 96-well plate and incubated at 37° C. for 4 hours. The wells were then washed 3 times with 1×PBS and placed in supplemented ENR media for the remainder of the culture. Supplemented ENR media was comprised of: basal organoid media plus 50 ng/mL EGF, 50 ng/mL Noggin (R&D Systems, 6057-NG-100), and 1 μg/mL R-spondin 1. Media was replaced every other day and put into antibiotic-free media the morning of the bacterial colonization experiments.

For protein staining, the samples were washed once with 1×PBS. After the wash, the samples were fixed with pre-warmed 4% PFA in 1×PBS for 10 minutes. The fixation solution was removed, and the samples incubated in 1×PBST containing 0.2% Triton X-100 for 10 minutes at room temperature. Monolayers were then blocked in 1% BSA in 1×PBS for 30 minutes with shaking, before being incubated in staining solution containing 300 nM DAPI, 1 mg/mL BSA, 0.66 μM Alexa Fluor 647 phalloidin (Invitrogen, A22287) and 1 M of FITC-labeled 57-aa peptide in 1×PBS. The monolayers were washed with 1×PBS twice before imaging. The samples were imaged with a spinning disk confocal microscope using a 60× water objective and a 405 nm laser excitation to observe monolayer nuclei, a 488 nm laser excitation to observe protein localization, and a 647 nm laser excitation to observe actin, with the corresponding filters.

### Staining and Visualization of Jejunum Tissue Slices

Pre-fixed human jejunum tissue slices were obtained from Novus Biologicals (NBP2-30201). Prepared slides were deparaffinized according to the manufacturers protocol. Briefly, the slides were dried for 1 hr at 60° C. and then soaked in xylene 5×4 minutes. The slides were then hydrated in 100%, 95%, and 75% ethanol 2×3 minutes and immersed in water for 5 minutes. Staining solutions containing 4 g/mL FM-4-64, 300 nM DAPI, 1 mg/mL BSA and 1 μM of FITC-labeled 57aa peptide in 1×PBS were added to the slides and incubated for 30 minutes at room temperature. Slides were carefully washed twice with 1×PBS. The samples were imaged with a spinning disk confocal microscope using a 60× water objective and a 405 nm laser excitation to observe the intestinal cells' nuclei, a 561 nm laser excitation to observed cell membranes, and a 488 nm laser excitation to observe the protein localization, with the corresponding filters.

### Aggregation Assay

Two stock solutions, 0.02% (weight percent) 200 nm carboxylate-modified microspheres (ThermoFisher F8807) in MilliQ water, and 150 μM FITC-labeled 57aa (Atlantic Peptides) in DMSO, were prepared. In an Eppendorf tube, 1 μL of 200 nm beads stock was added to 97 μL of M9 media with 0.2 mg/ml BSA (Sigma-Aldrich A9418), briefly vortexed, followed by 2 μL of FITC-labeled peptide stock, and briefly vortexed again, creating a bead solution with 0.0002% beads and 3 μM peptide. The sample was wrapped in aluminum and gently shaken for 1 hr at room temperature. Afterwards, it was transferred to a NaOH-treated 96-well plate with a glass bottom (MatTek P96G-1.5-5-F) and allowed to settle at room temperature for 5 min before imaging. The prepared samples were imaged with a spinning disk confocal microscope (Nikon Ti2-E connected to Yokogawa W1) using a 60× oil objective (numerical aperture=1.40) and laser excitation at 488 nm, 640 nm and bright field. Z-series images were captured with a sCMOS camera (Photometrics Prime BSI) and converted to 3D images in NIS-Elements.

E. coli Expression

The 57-aa peptide sequence was PCR amplified and cloned into the pET-28b expression plasmid (Novagen) using the NdeI and XhoI restriction sites, which includes a 6-His tag followed by a thrombin cleavage site. For expression without the his-tag, the 57-aa sequence was PCR amplified and cloned into the NcoI and XhoI restriction sites in pET-28b. Following an overnight culture, expression was carried out in T7-Express cells (New England Biolabs) until an OD₆₀₀ of 0.6 was reached. 1 mM IPTG was added and cells allowed to express for 4 hours at 37° C. Cells were collected by centrifuging for 15 minutes at 4000×g, resuspended in TBS (20 mM Tris, pH 7.6, 150 mM NaCl) and disrupted using an Avestin Emusiflex-C5 high pressure homogenizer. Inclusion bodies were collected by centrifuging for 40,000×g for 30 minutes and washed once in a buffer containing TBS, once in TBS containing high salt and Triton X-100 (TBS with 3 M NaCl and 0.1% Triton X-100), once again with TBS. Inclusion bodies were solubilized in 2-8M Urea and centrifuged at 40,000×g for 30 minutes to remove unsolubilized material.

### Results

### Microbeads-Based Adhesion Assay.

Chemically synthesized, high-purity (>95%) 57-aa peptides with the native sequence have been produced, both unlabeled and N-terminal labeled with fluorescein isothiocyanate (FITC). A protocol was developed to characterize their physical adsorption to silica beads and visualized using a spinning disk confocal microscope. An image analysis procedure was also developed to quantify the excess of fluorescence signal on the bead surface over the solution signal (FIG. 9A). By varying the concentration of the peptide in the solution and repeating the measurements, the Langmuir adsorption curve was mapped for the native 57-aa sequence (FIG. 9A). Compared to the FITC control, the 57-aa sequence had a clear, strong tendency to coat the silica beads. The adsorption is quick and irreversible: the signal does not change after 5 minutes of adsorption and washing or sonication does not detach the 57-aa peptide from the surfaces. these measurements were repeated for different pH values and ionic strengths and the results do not depend on these external factors. In addition to the microscopy-based adsorption measurement, these results were also confirmed using flow cytometry (FIGS. 9B-9C).

### Effect of Sequence Variation on Adhesion

Chemically synthesized, high-purity peptides with various designed sequences, both unlabeled and N-terminal labeled with fluorescein isothiocyanate (FITC) was obtained. Their physical adsorption to silica beads with modified and unmodified surfaces was characterized. A list of tested sequences is shown in FIG. 9D. Data shown in FIG. 9E and FIG. 9F indicate that the middle sequence WFFG (SEQ ID NO:46), flanked by the repeating motifs, plays a key role in adhesion to various surfaces, while the repeating motif at the periphery plays accessory roles. As shown in the preliminary data (FIGS. 9E-9F), removing aromatic residues such as tryptophane and tyrosine in the repeating motif in the periphery leads to a loss in biofilm adhesion to glass substrate. Meanwhile, removing the aromatic central region of the sequence decreases, but does not eliminate glass adhesion. Finally, removing the flexible linkers between the repeating motifs abolishes adhesion to glass, implicating that certain flexibility is important to allow the repeating units to simultaneously contact the glass substrate.

Adhesion Assay on Surfaces with Various Properties

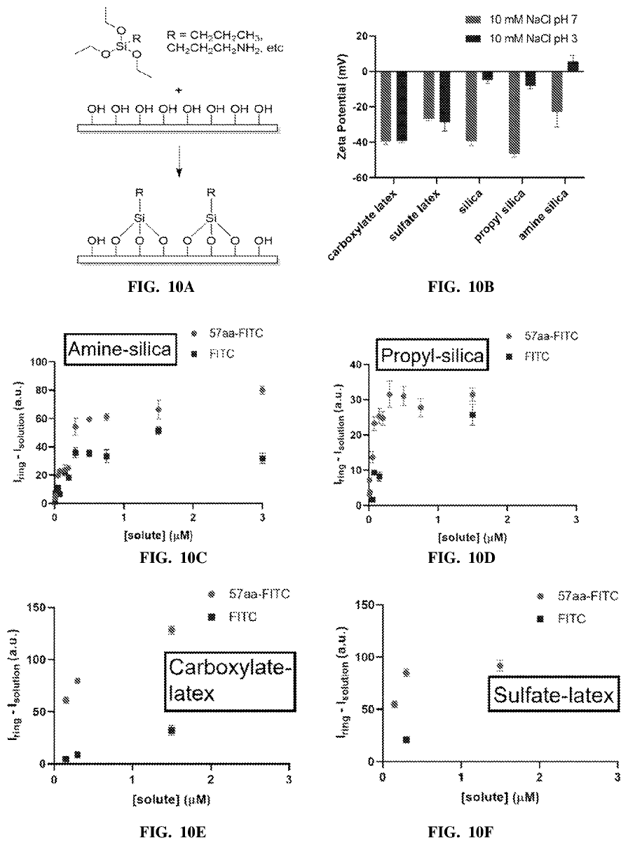

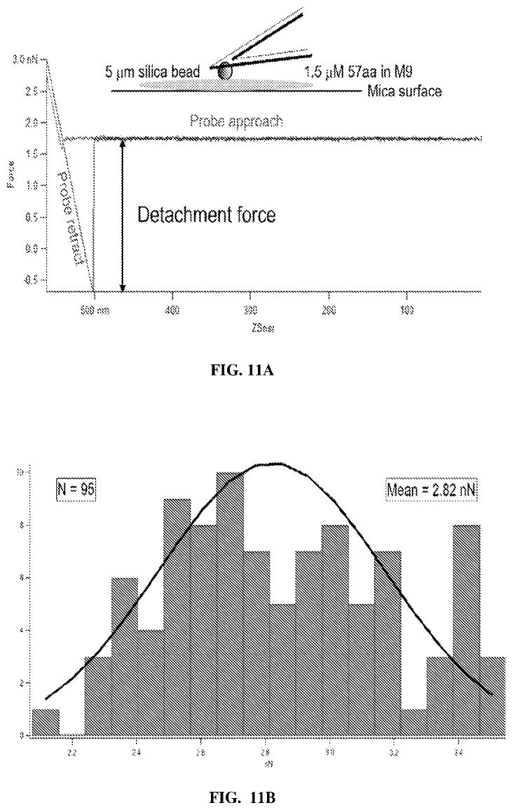

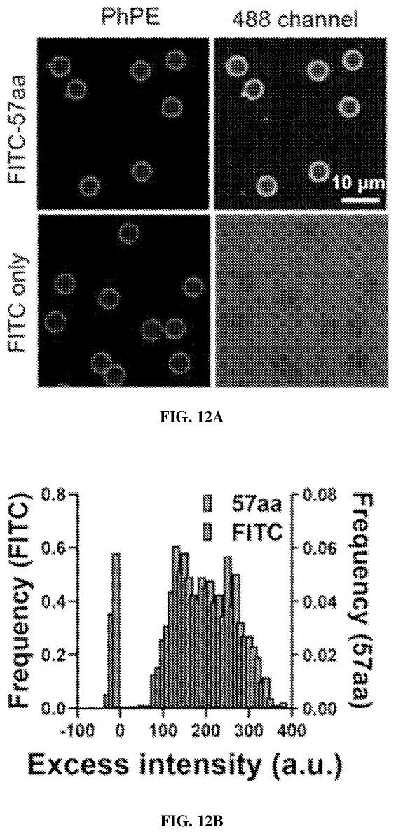

To demonstrate the broad applicability of the 57-aa sequence in adhering to various surfaces, the Langmuir curves (Israelachvili, Academic Press, St Louis, Missouri, U.S.A.) were repeated on various surfaces with different chemistry. Silane-based chemistry (Xu, et al., Proc. Natl. Acad. Sci. USA 107, 14964-14967 (2010)) (FIG. 10A) was used to functionalize the silica with different chemical groups including short alkyl chains and amine, to generate surfaces with different charges and hydrophobicities; in parallel, hydrophobic polystyrene (latex) beads were purchased with different surface charges to further strengthen the conclusions. The surface charge density of the beads was quantified using zeta potential measurements (FIG. 10B). In general, much stronger adsorption of the 57-aa peptide was observed onto hydrophobic surfaces (FIGS. 10C-10F). When the surface hydrophobicity is kept constant, the adsorption propensity increases with increasing magnitude of the negative charges on the surface. Together, the results point to a synergetic role between the positively charged lysine and the hydrophobic aromatic groups (tyrosine, tryptophane, etc.) in adhering to various abiotic surfaces commonly encountered in applications.

### Atomic Force Microscopy

To quantitatively measure the adhesive strengths of the 57-aa peptide, atomic force microscopy was used (Viljoen, et al., J Bacteriol 202, (2020)). Briefly, a silica microbead was attached to the cantilever of the AFM probe coated with the 57-aa peptide. Subsequently, the probe was operated to approach the mica substrate, also precoated with the 57-aa peptide, and dwell for 5 seconds in the initial test (FIG. 11A). Subsequently, the bead was separated from the mica surface and the detachment process was recorded; the detachment force corresponds to the maximum adhesive strength of the glue F_max. F_max is related to W, the work of adhesion, through F_max=3/2πRW, in which R is the radius of the bead (Israelachvili, Academic Press, St Louis, Missouri, U.S.A.). Many measurements (N=95) were performed to give a distribution of F_max (FIG. 11B). The peak value of F_max (2.82 nN) can be converted to a W of 0.24 mJ/m2. Although this value is smaller than what have been measured in mfps (Narayanan & Joy, Chem. Soc. Rev. 50, 13321-13345 (2021), Waite, J. Expt. Biol. 220, 517-530 (2017)) a direct comparison is difficult due to differences in the measurement geometry and amino acid composition. Also, a rather short dwell time was used in the initial experiment and work in the field of mfps implies a maturation effect in which a longer dwell time leads to stronger adhesion (Gebbie, et al., Nat. Chem. 9, 473-479 (2017), Danner, et al., Biochemistry 51, 6511-6518 (2012)).

Other measurements were also performed including circular dichroism (CD) to investigate the conformation of the peptide in the solution (Garcia Quiroz, et al., Science Advances 5, eaax5177). The melting behavior of the peptide was also measured by varying the temperature in the CD measurements.

### Adhesion of the 57-Aa Peptide to Lipids.

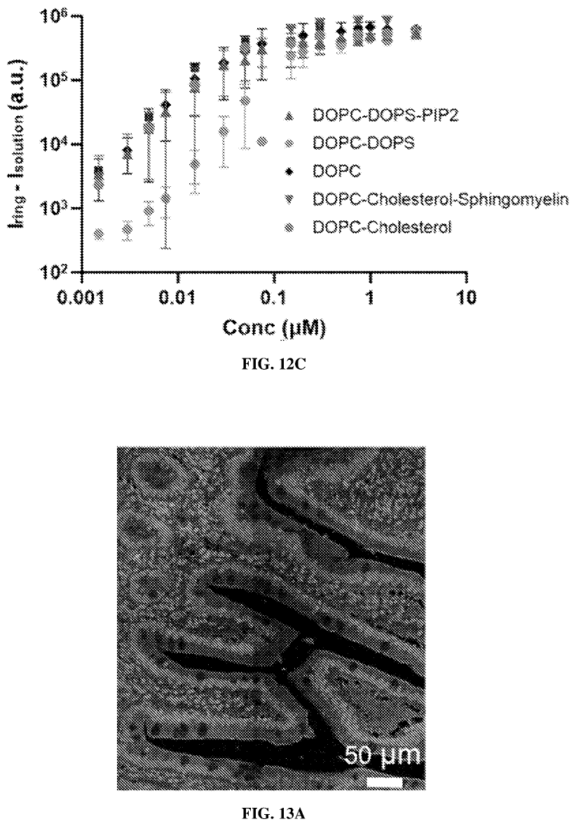

To explore the range of surfaces that the biofilm-derived peptide could adhere to, silica beads were further coated with lipids of various compositions and the adhesion assay was repeated. Indeed, in preliminary experiments, strong adsorption of the peptide to a commonly used lipid composition of 75% PC and 25% PI was observed (FIG. 12A). Computer algorithms were developed to quantify the fluorescent signal on the lipid-coated beads, which is proportional to the number of adsorbed molecules (FIG. 12A). To systematically study the adsorption kinetics and thermodynamics of the peptide on lipids, the surface signal was plotted as a function of the concentration of the peptide in the solution. FIG. 12C shows such adsorption curves for different lipid compositions. Preliminary results demonstrate that the biofilm-derived peptide can adhere to a wide range of lipid compositions, and the various parts of the sequence play complementary roles in lipid adhesion.

Using the same set of bacterial mutants described in FIG. 9D, the effect of sequence variation on lipid binding was also tested. Biofilms with different loop sequences are incubated with fluorescently labeled SUVs, and subsequently subjected to washing. Both the aromatic unit at the center of the sticky loop and the repeating units at the periphery are important for lipid binding. Results from in vitro experiments with chemically synthesized peptides have confirmed some of these conclusions.

### Adhesion of the 57-aa Peptide to Cell Surfaces.

The ability of the 57aa to adsorb on lipids suggests that it may also adhere to the plasma membrane of epithelial cell surfaces. Indeed, the FITC-labeled 57-aa peptide stains the entire cell surface of human intestinal epithelial slices (FIG. 13A), as well as the surface of a mouse enteroid monolayer (FIG. 13B). These results indicate that the 57-aa peptide not only binds to abiotic surfaces but also to important biotic surfaces such as those in humans, making it suitable for future biomedical applications in vivo.

### Flocculation Assay

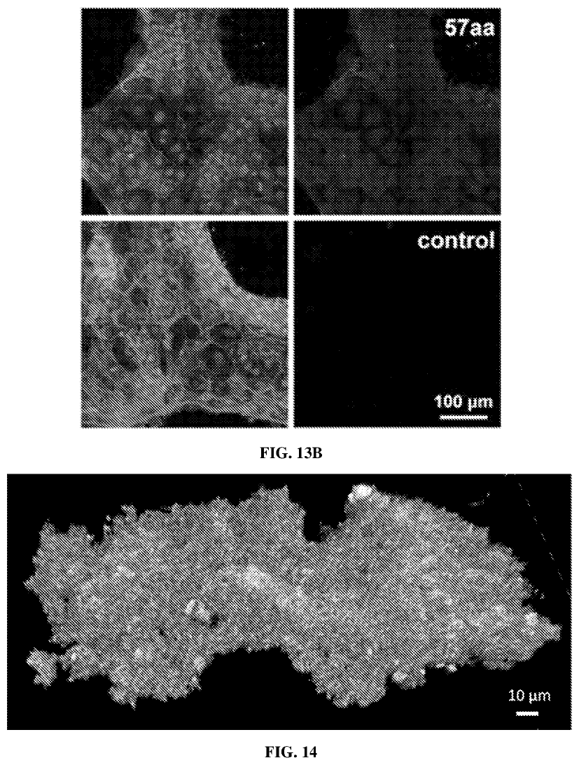

In the above experiments, a strong tendency of the 57-aa peptide to aggregate in aqueous solution was noticed. The tendency of the 57aa peptide to both aggregate on its own and to adsorb on particles renders it a good flocculant for applications such as wastewater treatment and chemical purification, in which particles in aqueous solutions need to be removed or recycled. To demonstrate this idea, the 57-aa peptide was mixed with 200 nm fluorescent polystyrene particles (FIG. 14). The formation of large aggregates was observed in which the peptide and the particles are intertwined, and the number of particles in the solution significantly dropped. Such mixed aggregates were not observed in samples with peptide or particles alone. This result demonstrated the potential of the 57aa peptide in removing suspended particles from a solution.

Strategies for E. coli Expression of the 57-Aa Peptide

Initial attempts to express the entire Bap1 gene, or the isolated β-prism domain in E. coli led to insoluble material. Bap1 was only expressed in a soluble form after removal of the 57-aa peptide. A fusion of the peptide to green fluorescent protein (GFPUV), also resulted in insoluble material. This fusion protein was isolated and solubilized from inclusion bodies using urea but attempts to refold the construct always led to insoluble material. It would be difficult to proteolytically cleave the 57-aa peptide from GFP in the presence of urea. To address this, new strategies have been designed:

### Express by Itself

To demonstrate the feasibility of large-scale production of the biofilm-derived peptide, E. coli expression constructs containing only the 57-aa sequence was designed, fused to a polyhistidine tag, and a pelB secretion tag (and his tag), which is a signal that directs proteins to the periplasm. A pelB leader was also placed on the original GFP_{UV} fusion construct to see if periplasmic targeting helps solubility.

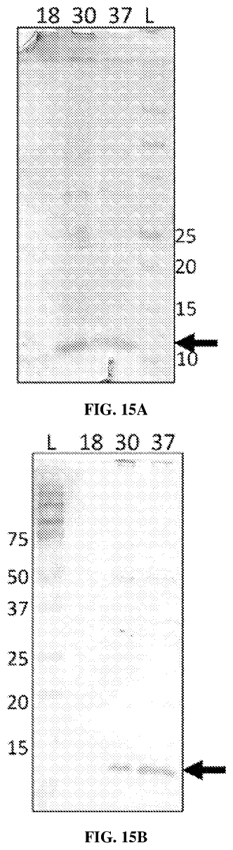

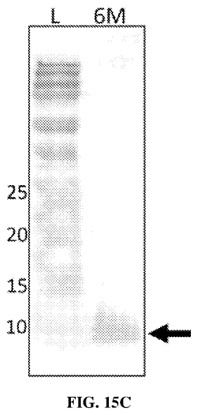

For example, proteins were in E. coli from pET28B with and without a his tag. When expressed in E. coli, the 57-aa peptide (with and without His tag) accumulates in inclusion bodies and remains in the insoluble fraction following cell disruption and centrifugation. A Western-blot using an anti-polyhistidine tag antibody demonstrates successful expression of the peptide in this way (FIG. 15). It was demonstrated that the washed inclusion bodies can be solubilized in various amounts of urea (2M-8M) as well as DMSO. At least tens of milligrams of solubilized inclusion body material per liter of E. coli culture can be produced in this way.

Other strategies for protein expression and purification are also contemplated as discussed below and elsewhere herein.

Express by Itself with a pelB Secretion Tag

E. coli expression constructs can also be made of the 57-aa sequence can also be made fused to a pelB secretion tag (and his tag) which is a signal that directs proteins to the periplasm (e.g., SEQ ID NO:34), etc. A pelB leader was can also be put on the GFP_{UV} fusion construct to see if periplasmic targeting helps solubility.

Express as a Fusion with a Large, Well-Behaved Partner:

The 57-aa sequence can also be attached to the end of a fragment of a well-behaved protein (FrhA). The idea is that the larger protein will improve the solubility of the small peptide. Next, an additional proteolytic site between FrhA and the 57-aa sequence will be added to cleave off the 57-aa peptide.

Expression as a Fusion with a Pore-Forming Toxin

Vibrio cholerae cytolysin (VCC) (De & Olson, Proc. Natl. Acad. Sci. USA 108, 7385-7390 (2011)), a pore-forming toxin, can be expressed in E. coli. Pore-forming toxins are secreted in a water-soluble state, but then bind to membranes and form a transmembrane channel. So, an important feature of their function is that in the water-soluble state, they can shield a hydrophobic loop from the solvent and keep it soluble in solution. This hydrophobic loop is to be replaced with the 57-aa sequence to see if VCC can act as a molecular chaperone to produce soluble material, and to engineer in protease sites to release the peptide following purification. There are other pore-forming toxins that may also be used—some of them can be triggered to assemble in solution using detergents or other small molecules. This could potentially lead to a protein that is expressed in a soluble form but can be triggered to attach to a surface by exposure of the 57-aa loop. In effect, this would be a triggerable glue that might have interesting applications in industrial or biomedical settings.

### Extracellular Secretion

The original protein Bap1 in which the 57-aa sequence is found in a secreted protein (Johnson, et al., J. Bacteriol. 196, 4245-4252 (2014)). Therefore, E. coli strains can be constructed in which the 57-aa is attached to an extracellularly secreted protein.

### Summary

In a serendipitous discovery while studying how Vc biofilms adhere to surfaces, a short protein sequence made of 57-amino acids was discovered that is majorly responsible for Vc adhesion to various abiotic surfaces, including glass and plastics (FIG. 1). Through further investigation of the purified peptide and variations of the original sequence, this is an effective, generic, and readily manipulatable peptide for applications involving underwater adhesion. Because of its bacterial origin and the absence of posttranslational modifications, it is possible to mass produce through fermentation, paving the way for scaling up its production for industrial or biomedical use. This sequence is also more stable under various pH conditions and insensitive to oxidation.

These findings are unusual because this sequence was discovered when studying Vibrio cholerae, a human pathogen that causes the pandemic cholera (Nelson, et al., Nat. Rev. Microbiol. 7, 693-702 (2009), Teschler, et al., Nat. Rev. Microbiol. 13, 255-268 (2015)). A sequence derived from this pathogen could be used for industrial applications. The sequence is not associated with Vc pathogenicity and the sequence is also present in other Vibrio species that are not human pathogens, so it should be safe. The simplicity in manipulating peptide sequences in bacteria allows rapid testing and screening for best sequences for particular applications (i.e. a particular surface to which a desired protein is targeted). Also, the sequence can be easily integrated into other recombinant proteins to direct the protein to adhere to surfaces. The adhesion function of this peptide is unaffected by the properties of the surface including hydrophobicity and charge, which is advantageous for wide range of applications.

Unless defined otherwise, all technical and scientific terms used herein have the same meanings as commonly understood by one of skill in the art to which the disclosed invention belongs. Publications cited herein and the materials for which they are cited are specifically incorporated by reference in their entireties.

Those skilled in the art will recognize, or be able to ascertain using no more than routine experimentation, many equivalents to the specific forms of the invention described herein. Such equivalents are intended to be encompassed by the following claims.

We claim:

1. An adhesive polypeptide comprising an adhesive domain comprising an amino acid sequence of SEQ ID NO:1 or 4, or a functional fragment or variant thereof, and does not comprise 100% sequence identity to the entire amino acid sequence of SEQ ID NO:2 or 6, wherein the polypeptide adheres to a surface under aqueous conditions.

2. The adhesive polypeptide of claim 1, further comprising one or more additional heterologous amino acid sequences.

3. The adhesive polypeptide of claim 1 or 2, wherein the polypeptide is complexed to, complexed with, or is otherwise associated with one or more additional heterologous molecule(s).

4. The adhesive polypeptide of claim 2 or 3, wherein the heterologous molecule is selected from the group consisting of an amino acid, a protein, a nucleic acid, a carbohydrate, a lipid, a metal, a polymer, a cell, a virion, a small molecule, and a mineral, or combinations thereof.

5. The adhesive polypeptide of any one of claims 2, 3 or 4, wherein the heterologous molecule improves a physicochemical property of the polypeptide selected from the group consisting of solubility, adhesion force, cross-linking, and improvement in protein expression, purification, recovery rate, and biodegradability of the adhesive protein

6. An adhesive polypeptide comprising an adhesion domain comprising at least 70% and less than 100% sequence identity to SEQ ID NO:1 or 4, or functional fragment thereof,
wherein the polypeptide adheres to a surface under aqueous conditions,
optionally further comprising a heterologous amino acid sequence and/or a heterologous molecule,
optionally wherein the polypeptide comprises the amino acid sequence
(SEQ ID NO: 37)
YLGLEWATATVPYLGVEWATATVSYWFFGWATAQVAYLAPVWAEATIPY
AVPVTLSK,
(SEQ ID NO: 38)
YLGLELKTKTVPLLGVELRTKTVSLWFFGLHTKQVALLAPVLKEKTIPL
AVPVTLSK,
or
(SEQ ID NO: 39)
YLGLEWKAKAVPYLGVEWRAKAVSYWFFGWHAKQVAYLAPVWKEKAIPY
AVPVTLSK.

7. The adhesive polypeptide of claim 6, wherein the variant or fragment is between 25 and 70 amino acids inclusive, or any subrange or specific integer therebetween.

8. The adhesive polypeptide of any one of claims 1-7, wherein the heterologous amino acid sequence and/or heterologous molecule comprises one or more purification tags and/or a second adhesion domain optionally wherein the second adhesion domain adheres to biotic surfaces.

9. The adhesive polypeptide of any one of claims 1-8, wherein the heterologous amino acid sequence and/or heterologous molecule comprises second adhesion domain optionally wherein the second adhesion domain adheres to biotic surfaces.

10. The adhesive polypeptide of any one of claims 1-9, wherein the variant comprises at least 75% sequence identity of SEQ ID NO:1 or 4, or a functional fragment thereof.

11. The adhesive polypeptide of any one of claims 1-10, wherein the polypeptide adheres to an abiotic surface in an aqueous environment.

12. The adhesive polypeptide of claim 11, wherein the abiotic surface comprises a material selected from the group consisting of metal, stone, plastic, glass, silica, concrete, paint, carbon, rubber, ceramic, and polymer fabric, or combinations thereof.

13. A polypeptide having an amino acid sequence that is at least 75 to 99% identical to SEQ ID NO:1 or 4, wherein one or more residues are mutated compared to SEQ ID NO:1 or 4, and wherein the polypeptide does not adhere to a surface
optionally wherein the polypeptide comprises the amino acid sequence
(SEQ ID NO: 40)
YLGLEAKTKTVPALGVEARTKTVSAWFFGAHTKQVAALAPVAKEKTIPA
AVPVTLSK,
(SEQ ID NO: 41)
YLGLEWKTKTVPYWRTKTVSYWHTKQVAYWKEKTIPYAVPVTLSK,
(SEQ ID NO: 42)
YLGLEWKTKTVPYLGVEWRTKTVSYLGPEWHTKQVAYLAPVWKEKTIPY
AVPVTLSK,
(SEQ ID NO: 43)
WKTKTVPY,
(SEQ ID NO: 44)
LGLEWKTKTVPYLGVEWRTKTVSY,
(SEQ ID NO: 45)
VSYWFFGWHTK.

14. The polypeptide of claim 13, wherein the mutation(s) comprises one or more one or more Lysine to Alanine.

15. A nucleic acid encoding the polypeptide of any one of claims 1-14 and a heterologous nucleic acid sequence.

16. The nucleic acid of claim 15, wherein the nucleic acid is RNA or DNA.

17. The nucleic acid of claim 15 or 16, wherein the nucleic acid comprises a vector, such as an expression vector.

18. A cell comprising the nucleic acid of claim 15, wherein the nucleic acid sequence is heterologous in the cell.

19. The cell of claim 18, wherein the cell expresses a polypeptide comprising the amino acid sequence of SEQ ID NO:1 or 4.

20. The cell of claim 18 or 19, wherein the cell is selected from the group consisting of a bacterium, a protozoan, a plant cell, an insect cell, a mammalian cell, a yeast cell and a fungal cell.

21. A composition comprising the adhesive polypeptide of any one of claims 1-12, wherein the polypeptide forms an adhesive layer within or on the composition.

22. The composition of claim 21, wherein the adhesive layer is in contact with a surface, and wherein the adhesive layer adheres the composition to the surface.

23. The composition of claim 22, wherein the surface comprises a material selected from the group consisting of metal, stone, plastic, glass, silica, concrete, polytetrafluoroethylene, paint, carbon, rubber, ceramic, and polymers, or combinations thereof.

24. The composition of any one of claims 21-23, wherein the adhesive polypeptide comprises between 0.1% and 50% by weight of the total composition.

25. An adhesive glue, comprising
(i) the adhesive polypeptide of any one of claims 1-12; and
(ii) an excipient,
wherein the adhesive polypeptide is suspended or mixed within the excipient; and
wherein the adhesive polypeptide comprises between 0.1% and 99.9% by weight of the glue.

26. The glue of claim 25, wherein the adhesive polypeptide comprises between 1% and 50% by weight of the glue.

27. The glue of claim 25 or 26, wherein the glue is in the form of a liquid, a gel, an emulsion, a cream, an aerosol, a powder or a foam.

28. The glue of any one of claims 25 to 27, wherein the excipient is not an aqueous solution.

29. The glue of any one of claims 25 to 28, wherein the excipient is selected from the groups consisting of a surfactant, an oxidant, and a filler,
optionally wherein the filler is selected from the group consisting of collagen, hyaluronic acid, condroitan sulfate, elastine, laminin, caseine, hydroxyapatite, albumin, fibronectin, and hybrin.

30. A coating material comprising the glue of any one of claims 25-29.

31. A container comprising the glue of any one of claims 25-29, or coating material of claim 30,
wherein the container comprises means for extrusion of the glue or coating material, and
wherein the glue or coating material does not adhere to the inside of the container.

32. A method of adhering two or more compositions together, comprising
(i) contacting a first composition with the adhesive polypeptide of any one of claims 1-12, or with the glue of any one of claims 25-29, to form a first adhesive composition comprising an adhesive layer;
(ii) contacting a second composition with the first adhesive composition,
wherein the contacting comprises interaction between the second composition and the adhesive layer, sufficient for the first adhesive composition and second composition to adhere together.

33. The method of claim 32, wherein the contacting in step (i), or step (ii), or in both steps (i) and (ii) occurs in an aqueous environment.

34. The method of claim 32 or 33, wherein the adhesive layer comprises the adhesive polypeptide of any one of claims 1-12, or the glue of any one of claims 25-29 in an amount between about 0.01 μg/cm² and about 100 μg/cm², inclusive.

35. The method of claim 34, wherein the aqueous environment is underwater.

36. The method of claim 35, wherein the underwater environment is in an ocean or sea, or a lake, river, loch, reservoir or other body of water.

37. The method of any one of claims 32-34, wherein the aqueous environment is in or on the body of a subject.

38. The method of claim 37, wherein the subject is a human.

39. A method of removing a target molecule in a solution comprising contacting a solution comprising the target molecule with an effective amount of the adhesive polypeptide of any one of claims 1-12 for an effective amount of time to form an aggregate with the target molecule and removing the aggregate from the solution.

40. The method of claim 39, wherein the solution is an aqueous solution, and optionally wherein removing the aggregate comprises allowing the aggregate to settle by gravity and/or centrifugation, and/or optionally capturing the aggregate by affinity binding to a substrate the binds to the polypeptide.

41. The claim of paragraphs 39 or 40, wherein removing the aggregate comprises gravity settling, centrifugation, affinity binding, filtration, or a combination thereof.

42. The method of any one of claims 39-42, wherein the target material is selected from soil colloids, emulsion droplets, clay particles, plastic contaminant, algae cells, chitin particles, and particulate aggregates.
